(19) United States
(12) Patent Application Publication
(10) Pub. No.: US 2018/0286649 A1
(43) Pub. Date: Oct. 4, 2018

(54) Implementation of Continuous Wave Carbon Dioxide Infrared Laser on a Quadrupole-Orbitrap-Linear Ion Trap Hybrid Mass Spectrometer System

(71) Applicant: Wisconsin Alumni Research Foundation, Madison, WI (US)

(72) Inventors: Joshua COON, Middleton, WI (US); Nicholas RILEY, Madison, WI (US); Michael WESTPHALL, Fitchburg, WI (US)

(73) Assignee: Wisconsin Alumni Research Foundation, Madison, WI (US)

(21) Appl. No.: 15/936,288

(22) Filed: Mar. 26, 2018

Related U.S. Application Data
(60) Provisional application No. 62/477,406, filed on Mar. 27, 2017.

(51) Int. Cl. H01J 49/00; H01J 49/42; G01N 33/68

(52) U.S. Cl. CPC H01J 49/0059; G01N 33/6848; H01J 49/0027; H01J 49/425

(57) ABSTRACT

A new approach is described herein for outfitting a mass spectrometer with an infrared laser that provides an improved method of ion dissociation. One embodiment, generally referred to as Activated Ion Electron Transfer Dissociation (AI-ETD) utilizes additional energy from photons during fragmentation to generate extensive fragmentation by interacting with peptides or proteins that are not fully fragmented or separated in the high pressure linear ion trap, thus allowing for increased information during MS/MS. Additionally, a new activation scheme generally referred to as AI-ETD+ is also described that combines AI-ETD in the high pressure cell of the linear ion trap with additional infrared multi-photon dissociation (IRMPD) activation in the low pressure cell. These methods provide improved fragmentation and sequence coverage without introducing additional time to the scan duty cycle.

### CROSS-REFERENCE TO RELATED APPLICATIONS

This application claims priority from U.S. Provisional Patent Application No. 62/477,406, filed Mar. 27, 2017, which is incorporated by reference herein to the extent that there is no inconsistency with the present disclosure.

### STATEMENT REGARDING FEDERALLY SPONSORED RESEARCH OR DEVELOPMENT

This invention was made with government support under GM118110 and GM108538 awarded by the National Institutes of Health. The government has certain rights in the invention.

### BACKGROUND OF THE INVENTION

Photo-dissociation (PD) in tandem mass spectrometry (MS/MS) has been an accessible and robust tool for characterization of inorganic and organic molecular ions for decades. Despite their utility, PD methods have been less favored in proteomic technologies due to the cost and difficulty of implementing PD based methods into mass spectrometer systems especially compared to the ease of implementation of collision-based fragmentation methods. However, collisional activation has several shortcomings in peptide and protein sequencing, which has led to the development of alternative fragmentation methods, including electron-based dissociation.

In over a decade of availability on commercial mass spectrometers, electron transfer dissociation (ETD) has become a valuable and ubiquitous fragmentation method used in a wide range of proteomic experiments (Syka et al., Proc. Natl. Acad. Sci. U.S.A., 2004, 101: 9528-9533; Coon et al., J. Anal Chem, 2009, 81:3208-3215; Zhurov et al., Chem. Soc. Rev., 2013, 42:5014-5030; Prentice et al., Chem. Commun., 2013, 49:947-965; Sarbu et al., Amino Acids, 2014, 46:1625-1634; and Brodbelt et al., Anal. Chem., 2016, 88:30-51). Continued advancements in ETD instrumentation and methodology have contributed to this sustained utility, and many of these improvements have been incorporated into the newest generation of mass spectrometer systems. For example, the robustness of the ETD reagent ion sources has been improved (Earley et al., Anal. Chem., 2013, 85:8385-8390), modifications to precursor and reagent ion storage prior to the reactions has improved product ion signal-to-noise (S/N) (Riley et al., J. Am. Soc. Mass Spectrom., 2016, 27:520-531), and calibration routes have standardized ETD reaction times to balance sufficient precursor fragmentation with maximal identification power for proteomic analyses (Rose et al., J. Am. Soc. Mass Spectrom. 2015, 26:1848-1857).

Despite these gains, however, a major challenge of ETD remains in its reduced dissociation efficiency for precursor ions having low charge density. Low-charge density precursor ions are more compact and often undergo non-dissociative electron transfer (ETnoD), where peptide backbone bond cleavage occurs but fragments are held via together non-covalent interactions (Good et al., J. Mol. Cell. Proteomics, 2007, 6: 1942-1951; Lermyte et al., J. Am. Soc. Mass Spectrom., 2015, 26: 1068-1076; Laszlo et al., J. Am. Chem. Soc., 2016, 138: 9581-9588; Pitteri et al., Anal. Chem., 2005, 77: 5662-5669; Liu et al., Int. J. Mass Spectrom., 2012, 330: 174-181; Clemmer et al., J. Am. Chem. Soc. 1995, 117: 10141-10142; and Breuker et al., J. Am. Chem. Soc. 2002, 124: 6407-6420). ETnoD may impede the generation of sequence-informative product ions and thus limits the utility of ETD in standard shotgun experiments, where the majority of peptide precursors are doubly protonated. That said, ETnoD can be minimized in favor of sequence-informative product ions by supplying the ETD reaction with more energy, and several supplemental activation strategies have been implemented to accomplish this goal. The most ubiquitous approach has been the use of gentle collisional dissociation of ETnoD products, called ETcaD, which is effective at producing more fragmentation and sequence coverage of low charge-density precursors (Swaney et al., Anal Chem, 2007, 79: 477-485). Likewise, activation of all ETD product ions (including remaining precursor ions, fragment ions, and ETnoD products) with beam-type collisional dissociation, termed EThcD, has proven a valuable way to generate more extensive fragment ion series and sequence information from ETD reactions (Frese et al., Anal. Chem., 2012, 84: 9668-9673). For all of their merits, however, ETcaD and EThcD both suffer from fragment ions that have skewed isotope distributions due to hydrogen abstractions and, importantly, from increased cycle time due to the secondary activation step after completion of the ETD reaction (O'Connor et al., J. Am. Soc. Mass Spectrom., 2006, 17: 576-585; Sun et al., J. Proteome Res., 2010, 9: 6354-6367; and Xia et al., Anal. Chem., 2008, 80: 1111-1117).

A third alternative to increasing product ion yield in ETD reactions is concurrent photo-activation, i.e., activated ion ETD (AI-ETD). In AI-ETD, photons unfold peptide precursor ions by disrupting the non-covalent interactions via slow-heating, increasing product ion yield and the amount of sequence information obtained per spectrum (Ledvina et al., Angew. Chem. Int. Ed. Engl., 2009, 48: 8526-8528). It has been previously shown that AI-ETD can improve proteome characterization in shotgun experiments while minimizing hydrogen rearrangements in product ions (Ledvina et al., Anal. Chem., 2010, 82: 10068-10074; and Ledvina et al., J. Am. Soc. Mass Spectrom., 2013, 24: 1623-1633). Notably, AI-ETD accomplishes this without any addition to the standard ETD reaction time, making it highly favorable for high-throughput experiments that look to maximize the number of peptides sequenced per unit time.

However, AI-ETD has largely been unfeasible due to the problems associated with integrating lasers into the mass spectrometer system. For example, previous implementation of AI-ETD on an Orbitrap system required extensive hardware modifications (Ledvina et al., J. Am. Soc. Mass Spectrom., 2013, 24: 1623-1633; Rose et al., J. Am. Soc. Mass Spectrom., 2013, 24: 816-827; and Riley et al., Anal. Chem., 2015, 87: 7109-7116). What is needed are improved mass spectrometer systems which are able to easily and reliably integrate the use of lasers in order to facilitate PD and higher fragmentation efficiency. Additionally, there is an interest in combining PD with other fragmentation methods to create hybrid approaches that offer comprehensive characterization of peptides and proteins.

### SUMMARY OF THE INVENTION

The present invention provides a new approach for outfitting a mass spectrometer with a laser, preferably with an infrared (IR) laser, which provides an improved method of ion dissociation through the use of unique laser beam delivery system. In an embodiment generally referred to herein as Activated Ion Electron Transfer Dissociation (AI-ETD), the fragmentation method utilizes additional energy from photons to generate extensive fragmentation by interacting with peptides or proteins that are not fully fragmented/separated in the high pressure linear ion trap, thus allowing for information-rich MS/MS. In a further embodiment, AI-ETD performed in the high pressure cell of the linear ion trap is combined with additional infrared dissociation activation in the low pressure cell. Methods provided herein improved fragmentation and sequence coverage without introducing additional time to the scan duty cycle.

The present invention enables improved characterization of several classes of biomolecules. In an embodiment, use of an IR laser during fragmentation, optionally in conjunction with other activation methods such as electron transfer dissociation (ETD), results in increased fragmentation of unmodified peptides, phosphorylated peptides, glycosylated peptides, and intact proteins (both modified and unmodified with various post-translational modifications). Moreover, use of the laser during fragmentation translates to whole-proteome scale analyses and increases the information obtained from complex biological samples. Improved fragmentation and quality of spectra afforded by the implementation of the laser as disclosed herein offers dramatically greater analytical power in sequencing and identifying these biomolecules, a central tenant to modern protein sequencing technology.

An embodiment of the present invention provides a mass spectrometer device for analyzing a sample, where the device comprises: a) an ion source for generating ions from the sample; b) one or more chambers having an inlet for receiving the ions and having ion pathway optics for transmitting the ions along an ion injection pathway, c) a mass analyzer in fluid communication with the ion injection pathway; d) a linear ion trap in fluid communication with the ion injection pathway; and e) an optical assembly positioned external to the linear ion trap and the ion injection pathway, where the optical assembly is able to provide a photon beam through the beam entrance window into the linear ion trap. The linear ion trap has a longitudinal axis, a first end proximal to the ion source, and a second end distal to the ion source. The linear ion trap also comprises a high pressure cell, a low pressure cell, and a beam entrance window positioned at the second end of the linear ion trap. In an embodiment, the mass analyzer is an orbitrap mass analyzer.

Preferably, the ion pathway optics comprise two or more multipole RF devices and one or more ion lens devices, wherein at least one of the multipole RF devices is provided between the inlet and the mass analyzer, and at least one of the ion lens devices comprises an aperture provided between the high pressure cell and the low pressure cell. In an embodiment the ion lens devices and multipole RF devices provide spatial focusing of ions in the ion injection pathway.

The optical assembly comprises an infrared (IR) laser and one or more optical elements selected from the group consisting of guiding mirrors, waveguides, hollow silica waveguides, optical fibers, beam steerers, and focusing lenses able to provide and direct a photon beam through the beam entrance window into the linear ion trap. Preferably, the one or more optical elements comprise a hollow silica waveguide or an optical fiber able to transport photon beams from the IR laser to the linear ion trap. The photon beam has an optical axis which is substantially aligned along the longitudinal axis of the linear ion trap. In an embodiment, the photon beam is focused to a waist between approximately 0.5 to 2 mm in diameter and then columnated prior to entering the linear ion trap. Preferably, the IR laser is a continuous wave laser. In an embodiment, the IR laser has a power up to 60 watts, between approximately 6 to 30 watts, between approximately 12 to 24 watts, or a power of approximately 18 watts.

In a further embodiment, the device further comprises a controller operably connected to the ion injection pathway ion optics and optical assembly. The controller is able to control the ion injection pathway ion optics and optical assembly so as to: a) transmit the ions along a first direction away from the inlet through the ion injection pathway into the high pressure cell and low pressure cell of the linear ion trap; b) operate the IR laser to transmit the photon beam into the high pressure cell while ions are present in the high pressure cell, thereby fragmenting at least a portion of the ions to generate product ions; and c) transmit at least a portion of the generated product ions from the linear ion trap to the mass analyzer. In a further embodiment, the controller is able to control the ion injection pathway ion optics and optical assembly so as to further transmit unfragmented ions and at least a portion of the generated product ions from the high pressure cell to the low pressure cell; and operate the IR laser to transmit the photon beam into the low pressure cell while said unfragmented ions and generated product ions are present in the low pressure cell, thereby fragmenting additional ions to generate additional product ions. In an embodiment, the mass analyzer is an orbitrap mass analyzer.

The devices and methods described herein can encompass many different mass spectrometer systems. For example, the mass spectrometer device can include a series of differentially pumped vacuum chambers are provided between the ion source and the mass analyzer or linear ion trap. Each differentially pumped vacuum chamber can be defined by its own vacuum manifold.

The lenses included in the ion injection pathway are able to improve ion transmission between adjacent multipoles by establishing focal points for ions passing through the lenses and multipoles. Additionally, the ion lenses may comprise apertures that separate the differentially pumped vacuum chambers and help lower gas conductance between adjacent chambers while permitting ions to be transmitted between adjacent chambers. This helps to create discrete pressure ranges in each of the differentially pumped vacuum chambers, such as higher pressure chambers, for example greater than about 10 mTorr, and lower pressure chambers, for example less than about 10⁻⁴ Torr.

An embodiment of the present invention provides a method for generating product ions from a sample comprising the steps of: a) generating ions from the sample using an ion source; b) transmitting the ions from the ion source through an inlet into an ion injection pathway of a mass spectrometer device having ion pathway optics and a mass analyzer; c) transmitting the ions along a first direction away from the inlet through the ion injection pathway into a high pressure cell of a linear ion trap, wherein the linear ion trap has a longitudinal axis, a first end proximal to the ion source, and a second end distal to the ion source, and wherein the linear ion trap comprises the high pressure cell, a low pressure cell, and a beam entrance window, preferably positioned in the second end of the linear ion trap; d) transmitting a first photon beam from an external infrared (IR) laser through the beam entrance window into the high pressure cell while the ions are present in the high pressure cell, thereby fragmenting at least a portion of the ions to generate product ions; and e) transmitting at least a portion of the generated product ions from the linear ion trap to the mass analyzer of the mass spectrometer device.

In a further embodiment, the method further comprises the steps of: before transmitting the generated product ions to the mass analyzer, transmitting unfragmented ions and at least a portion of the generated product ions from the high pressure cell to the low pressure cell; and transmitting a second photon beam from the external IR laser through the beam entrance window into the low pressure cell while said unfragmented ions and generated product ions are present in the low pressure cell, thereby fragmenting additional ions to generate additional product ions. The present invention is beneficial in that the fragmentation steps using the photon beams from the external IR laser do not increase scanning time of the mass spectrometer device.

In an embodiment, the first photon beam is transmitted into the high pressure trap for a duration of approximately 5 to 200 ms while the second photon beam is transmitted into the low pressure trap for a duration of approximately 2 to 10 ms, preferably 4 to 6 ms.

The photon beam has an optical axis which is substantially aligned along the longitudinal axis of the linear ion trap and can be focused in the high pressure cell or the low pressure cell as necessary. Preferably, the IR laser is a continuous wave laser. In an embodiment, the IR laser has a power up to 60 watts, between approximately 6 to 30 watts, between approximately 12 to 24 watts, or a power of approximately 18 watts. Preferably, the second photon beam has lower power than the first photon beam. Preferably, the first photon beam has a power between approximately 12 to 24 watts, and the second photon beam has a power between approximately 8 to 10 watts.

In further embodiments, the IR laser and/or one or more optical elements selected from the group consisting of guiding mirrors, waveguides, hollow silica waveguides, optical fibers, beam steerers, and focusing lenses are rigidly mounted on the chassis is rigidly mounted on the device chassis which encompasses the one or more chambers, ion injection pathway, mass analyzer, and linear ion trap. Preferably, the photon beams are transmitted from the external IR laser to the beam entrance window through one or more hollow silica waveguides or optical fibers. Preferably, the device does not comprise an additional collision cell or ion trap between the mass analyzer and linear ion trap along the ion injection pathway. In an embodiment, the device further comprises a beam dampening barrier which prevents photon beams from passing out of the linear ion trap. Preferably the ion pathway optics comprise two or more multipole RF devices and one or more ion lens devices, wherein at least one of the multipole RF devices is provided between the inlet and the mass analyzer, and at least one of the ion lens devices comprises an aperture provided between the high pressure cell and the low pressure cell.

In an embodiment, the mass spectrometer device further comprises a separation stage operably connected to the ion source for fractionating the sample prior to generation of the ions. In an aspect, the separation stage is a liquid chromatography separation system or a capillary electrophoresis separation system.

Many ion sources are compatible with the devices and methods described herein. In an embodiment, for example, the ion source is an atmospheric ion source. In an aspect, the ion source is an electrospray ionization source, a MALDI source, a chemical ionization source, a laser desorption source, a sonic spray source, a photoionization source, a desorption source, or a fast ion bombardment source. In an aspect, the ion source is an electrospray ionization source or a MALDI source. In an embodiment, the ions are generated from proteins, peptides, small molecules, lipids, metabolites, or drugs.

In an embodiment, the ions are generated from a sample which is an unmodified, phosphorylated, glycosylated, or isobarically labeled protein or peptide. In a further embodiment, the ions are generated from a phosphorylated or glycosylated peptide. Optionally, the ions are generated from one or more peptides that are fractionated prior to generation of the ions. In an embodiment, the transmitted product ions provide a peptide backbone sequence coverage of at least 50%, preferably at least 60%, or preferably at least 75%. As used herein, peptide sequence coverage is defined as the number of inter-residue bonds accounted for by b-, c-, y-, and z-type fragment ions compared to the total number of inter-residue bonds in the peptide sequence, calculated as a percentage

In an embodiment, using concurrent IR photo-activation during electron transfer dissociation (ETD) reactions, i.e., activated ion ETD (AI-ETD), significantly increases dissociation efficiency resulting in improved overall performance. In an embodiment, the product ions are generated from a sample having a charge state of +2 or greater, a charge state of +3 or greater, a charge state of +4 or greater, or a charge state of +5 or greater.

In an embodiment, the present invention implements an IR laser on a q-OT-LIT (quadrupole-orbitrap-linear ion trap) mass spectrometer, the latest generation of Orbitrap hybrid instruments that provide remarkable improvements in acquisition speed, sensitivity, and proteomic depth (e.g., MS/MS acquisition rates double those of previous ion trap-Orbitrap hybrids). A CO₂ laser is implemented in a straight-forward manner by fastening the laser and guiding optics to the instrument chassis itself, making alignment with the trapping volume of the LIT simple and robust. This addition marries the immense gains in throughput and sampling depth afforded by the q-OT-LIT system with the versatile and robust fragmentation regimes offered via photo-dissociation. This invention will enable new paradigms that will improve characterization of a wide array of biomolecules, including peptides, proteins, and post-translational modifications.

### BRIEF DESCRIPTION OF THE DRAWINGS

FIG. 1 shows instrument schematics of a mass spectrometer device in different embodiments of the present invention. As illustrated in panel a, the device includes a CO₂ laser, guiding mirrors, beam steerer, focusing lenses, and an entrance window added to the vacuum manifold. Panel b illustrates a similar device where a hollow silica waveguide is instead used to transmit the photon beam to the ion trap.

FIG. 2 shows AI-ETD product ion generation over ETD, ETcaD, and EThcD. a) Increasing laser power enhances generation of c-, z●-, and y-type ions over ETD alone for four standard peptides, although too high of laser power can cause decreases in product ion intensity. Higher laser powers also begin to generate b-type ions, indicating onset of some degree of IRPMD. AI-ETD at 18 W and 24 W generally produces more product ion signal than ETcaD and EThcD supplemental activation techniques. b) Summing c- and z●-type product ion signal show how AI-ETD at varying laser powers increases ETD-specific product ions over ETD, ETcaD, and EThcD. Signal for each graph was normalized to the highest value for that condition.

FIG. 3 shows conversion efficiency of AI-ETD and other supplemental activation methods. The signal present in remaining precursor and charge-reduced precursor ion channels are presented for ETD (laser power 0), AI-ETD at varying laser powers, EThcD at 25 nce and ETcaD at 35 nce.

FIG. 4 shows AI-ETD results for higher charge states. a) Normalized product ion signal for the z=3 precursor of INQLISETEAVVTNELEDGR is calculated analogously to data in FIG. 2 (panel a), including both singly and doubly charge product ions. ETD preforms predictably better for the triply protonated precursor compared to the doubly charged peptide (FIG. 2), but AI-ETD still increases product ion generation, especially at lower laser powers. Above 18 W, over-fragmentation from the laser begins to degrade c- and z●-type product ion signal in favor of b- and y-type product ions. Even as AI-ETD improves spectral quality at lower laser powers, EThcD and ETcaD decrease product ion signal compared to ETD, highlighting the utility of AI-ETD a supplemental activation method suitable for all precursor ions, not only doubly protonated ions. b) Spectra from ETD and AI-ETD analyses of this triply protonated precursor show the increase in product ion signal with AI-ETD over ETD at lower laser powers in addition to the onset of over-fragmentation at higher laser powers.

FIG. 5 shows AI-ETD product ions generation by decreasing ETnoD. a) Spectra from ETD and AI-ETD of increasing laser powers for the z=+2 precursor of ENVNDDEDIDWVQTEK show the extent to which AI-ETD can increase product in yield. All spectra are on the same intensity scale. b) Investigation of the charge reduced precursor products (greyed area in spectra in part a) show that the isotopic distribution shift upon AI-ETD to match the presence of PTR products only with little to no ETnoD products. Base peak intensities for each case are provided. c) The base peak intensity of the PTR and ETnoD monoisotopic peaks for z=+2 precursors of SEDYVDIVQGNR, ENDILVLMESER, and ENVNDDEDIDWVQTEK are plotted to show that the trend of decreasing ETnoD products with increasing AI-ETD laser power holds for each of the standard peptides (the fourth peptide had a charge reduced precursor m/z above 2000 Th).

FIG. 6 shows AI-ETD at different laser powers compared to ETD for shotgun proteomics. a) AI-ETD across a range of laser powers improves peptide identification over ETD, with AI-ETD at 18 W generating the most peptide spectral matches—nearly doubling the identifications obtained from ETD alone. b) AI-ETD increases MS/MS success rate over ETD across the m/z range, especially >600 Th. c) AI-ETD improves MS/MS success rates over ETD for precursors of all charge states, with the exception of AI-ETD at 21 W for z≥5 (5+indicates z≥5). In general, AI-ETD at 18 W performs best for all metrics. The success rate is defined as number of MS/MS scans successfully converted to peptide spectral matches (PSMs), and all data here represent the average of three technical replicates with one standard deviation shown by error bars.

FIG. 7 shows AI-ETD+ offers maximal generation c-, z-, b- and y-type fragments. AI-ETD+ is the combination of irradiating ions in the high pressure trap concurrent with the ETD reaction and providing a second short irradiation during product ion analysis in the low pressure trap. This scheme does not increase the overall acquisition time per scan but provides the combination of benefits of AI-ETD fragmentation plus extra collisional excitation via IRMPD immediately prior to mass analysis. Spectra for the z=+2 precursor of INQLISETEAVVTNELEDGR are shown for AI-ETD at 18 W, EThcD with a collision energy of 25nce, and AI-ETD+(18 W during the ETD reaction and 9 W during product ion analysis). AI-ETD+ maintains the c- and z●-type product ion signal generated by AI-ETD and also provides more b- and y-type product ion signal than EThcD. Spectra are on the same intensity scale, and product ion signals were normalized to the highest value across all three conditions.

FIG. 8 provides graphs showing that AI-ETD+ is the optimal supplemental activation technique for shotgun proteomic analyses. a) Peptide spectral matches for ETD and four different ETD supplemental activation methods are shown, with AI-ETD+ providing the best identification power. MS/MS success rates for precursor m/z (b) and charge state (c) are also provided for each method. Note, data in a), b), and c) represent the average of three technical replicates, and error bars show one standard deviation. d) The number of missing bond cleavages is shown for the z=+2 precursors from EThcD, AI-ETD, and AI-ETD+. Zero missed cleavages indicates that 100% peptide sequence coverage was achieved for that PSM. e) Box plots show the distribution of total c-, z●, b-, and y-type product ions generated in a given PSM of z=+2 precursors for EThcD, AI-ETD, and AI-ETD+. Note, data in d) and e) represent the aggregate total of PSMs from all three technical replicates.

FIG. 9 shows scan sequences for AI-ETD+. Standard AI-ETD uses IR photo-activation only during the ion-ion reaction time of ETD. Following the AI-ETD reaction the laser is turned off and product ions are then shuttled from the high pressure trap (HPT) to the low pressure trap (LPT). Prior to mass analysis, the ions are held in the LPT for several ms, called settling time, and this is standard for all analyses using the linear ion trap. In AI-ETD+, the laser is turned on during the ETD reaction and turned off, as occurs with standard AI-ETD. Following product ion transfer into the LPT, however, the laser is turned on again (usually at a lower output power) only for the few ms of settling time immediately prior to mass analysis to generate IRMPD like products. The laser is then turned off again for mass analysis, ultimately adding no additional time to the instrument's duty cycle.

FIG. 10 illustrates the effect of fragment ion types on search results. The number of PSMs are reported for each of the ETD activation types when searched using only c-, z●-, and y-type products (i.e., “ETD only” fragments) or when using all four (c, z●, b, and y) fragment ion types. EThcD and AI-ETD+ perform better when using all four ion types, as is expected because they both aim to create collisional activated products in addition to ETD. The other methods performed better when including the “ETD only” fragment types, which also matches expectations. Note, AI-ETD at 21 W does perform better with all ion types, but does not outperform AI-ETD at 18 W, indicating a degree of over-fragmentation.

FIG. 11 illustrates the effects of activation on scan speed. a) The average number of MS/MS scans acquired for each activation type is shown with error bars representing one standard deviation. b) The elapsed scan time for each of the activation types is shown, with results behind delineated by precursor charge state. The difference between charge states occurs because of the charge dependent ETD reaction time settings used. ETcaD and EThcD have longer scan times than standard ETD because they require additional time to perform their supplemental activation steps. AI-ETD and AI-ETD+ maintain the same elapsed scan durations as standard ETD because activation is happening concurrently with ETD or occurring during times already inherent to the typical scan sequence.

FIG. 12 shows ETD and AI-ETD spectra of a phosphopeptide with several potential phosphosites. ETD produces moderate fragmentation of doubly protonated precursor of peptide SVSTpSPSILPAYLK but fails to confidently localize the modified phosphoserine (Xcorr 1.09, ˜46% backbone sequence coverage). Conversely, AI-ETD generates extensive fragmentation with c/z and b/y ion series to localize the phosphosite with high confidence (Xcorr 5.97, 100% backbone sequence coverage). Spectra are on the same intensity scale, and the grey regions are scaled to be ten percent of original intensity.

FIG. 13 shows performance of AI-ETD and other ETD activation methods for phosphoproteome analysis. a) The number of confidently localized phospho peptide spectral matches (phospho PSMs) for each activation method is shown, with singly-versus doubly-phosphorylated peptides being delineated (light grey versus dark grey, respectively). b) The median XCorr of PSMs and the number of unique phosphopeptides sequenced by each ETD activation methods is given. In (c) and (d), the distribution of peptide length (top) and peptide sequence coverage (bottom) is given for doubly (c) and triply (d) protonated peptides using ETD (gold) and AI-ETD at 15 W. Data represents confidently localized phospho PSMs. As used herein, peptide sequence coverage is defined as the number of inter-residue bonds accounted for by b-, c-, y-, and z-type fragment ions compared to the total number of inter-residue bonds in the peptide sequence, calculated as a percentage.

FIG. 14 shows successful AI-ETD fragmentation of phosphopeptides that challenge standard ETD. a) Precursor ion charge state distributions differ between whole proteome analysis and phosphopeptide-enriched samples, with the phosphoproteome shifting to higher charge states that should be favorable to ETD. b) The shift is charge states between whole proteome and phosphopeptide-enriched samples, however, is concomitant with a shift in precursor ion m/z distribution. Doubly protonated precursor ions (z=2) are largely 350-800 Th in whole proteome samples (dark grey), where ETD is more effective at dissociating peptides. The precursor ion m/z distribution in phosphoproteome samples is distinctly shifted to higher m/z values (light grey), which limits the success of ETD. A shift to higher m/z values in the phosphoproteome also occurs for triply protonated precursor ions (z=3), but the change is less dramatic. (c) The higher m/z values of phosphopeptides in this data set (light grey, part b) account for the low success rate of ETD on z=2 precursors. (d) In contrast, AI-ETD performs remarkably well for z=2 precursors compared to ETD and provides higher MS/MS success rates for all precursor charge states, which explains how the number of phospho PSMs sequenced can be more than tripled by AI-ETD over standard ETD (FIG. 13, panel a). As used herein, MS/MS success rate represents the percentage of MS/MS scans that were successfully sequenced at PSMs for precursors at a given charge state.

FIG. 15 shows that AI-ETD produces more sequence-informative product ions but also more phosphate losses from fragment ions. a) The average number of product ions generated per peptide spectral match (PSM) is shown for each of the ETD fragmentation conditions. The total number of product ions is delineated by ion type, and phosphate neutral losses are included in this count. b) The average percent of total ion current (TIC) that is accounted for by b-, c-, y-, and z-type product ions shows that AI-ETD and AI-ETD+ generate the most total signal for product ions, but more of this signal resides in phosphate neutral losses ions than in other supplemental activation methods. These values represent the average percent TIC accounted for by product ions per PSM. c) Percent phosphate retention indicates the proportion of all product ions (b-, c-, y-, and z-type) that retained their phosphate group out of all product ions detected, i.e., those with and without phosphate neutral losses. The calculation for this metric is provided, and the percent phosphate retention from HCD analyses of phosphopeptides is show with a dotted line. d) Percent phosphate loss is the complement to percent phosphate retention (% phosphate loss=100−% phosphate retention). Here, percent phosphate loss is calculated for each product ion type, showing that phosphate neutral losses from c- and z-type ions especially are a unique component of AI-ETD and AI-ETD+ spectra. All data in this figure is from doubly protonated precursors (z=2).

FIG. 16 shows that using phosphate losses from fragment ions improves phosphosite localization with AI-ETD. a) phosphoRS software was modified to use phosphate losses from product ions in ETD spectra to aid phosphosite localization. The percent gain in the number of localized phospho PSMs after modifying phosphoRS is shown for AI-ETD conditions. Using phosphate losses from different combinations of production types (c- and z-type ions only, b- and y-type ions only, and all four ion types) provides varying gains, with the biggest increase achieved when considering losses from all four ion types. b) The number of localized phospho PSMs for ETD, ETciD, EThcD, and AI-ETD conditions are compared when not considering any neutral losses in the phosphosite localization calculation or when enabling use of phosphate losses from product ions in ETD spectra.

FIG. 17 illustrates that AI-ETD enables confident characterization of intact phosphoprotein α-casein. a) AI-ETD provides the greatest sequence coverage of α-casein for all charge states investigated. b) The phosphor isoform of α-casein investigated had 8 phosphosites (out of nine previously reported sites). Only AI-ETD confidently localized all eight phosphosites for each precursor fragmented. The number of fragments (lines) and the percentage of these fragments that are b- and y-type product ions (bars) are shown from ETD (c), EThcD (d), and AI-ETD (e).

FIG. 18 shows comparisons between ETD and AI-ETD for the z=16 precursor of α-casein with 8 phosphosites. a) Full spectra of ETD and AI-ETD fragmentation show more signal in fragment ions and less signal in charge reduced precursor ions with AI-ETD, with AI-ETD providing significantly more matching fragments and protein sequence coverage. Both spectra are on the same intensity scale. b) A zoom in of the grey highlighted region of the spectra in part a show that AI-ETD generates more product ions with higher abundance than ETD. Increased product ion signal in AI-ETD also provided easy deconvolution of c45-c47 product ions that were crucial in localizing phosphosite S46. Low signal-to-noise in the ETD spectrum prevented identification of two of these three product ions. These spectra are also on the same intensity scale. c) A sequence coverage map of α-casein using AI-ETD shows confident localization of all 8 phosphoserines. This product ion map is from the AI-ETD spectrum in part a. Dark grey marks denote c- and z-type ions while light grey show b- and y-type products

FIG. 19 shows comparisons between the overlap of HCD and ETD-based methods. a) The table provides the number of localized phospho PSMs and unique phosphopeptides identified with HCD. The median Xcorr of localized phospho PSMs from HCD analyses is also provided. b) Phosphate neutral loss ions are common in HCD spectra and losses from b- and y-type ions are used to help localize phosphosites in phosphoRS (gold). If these neutral losses in HCD spectra are not accounted for by the localization software, the number of confidently localized phospho PSMs decreases by 17% (grey). Panels c-f) show the overlap in unique phosphopeptides identified with HCD and ETD (c), ETciD (d), EThcD (e), or AI-ETD (15 W) (f).

FIG. 20 shows overlap in unique phosphopeptides identified with ETD, EThcD, and AI-ETD (15 W). AI-ETD identifies the large majority of phosphopeptides observed with the other two methods and adds more than 1,400 additional unique phosphopeptides to the identification pool.

FIG. 21 shows sequence coverage maps from ETD and AI-ETD for the z=16 precursor of α-casein.

FIG. 22 shows a comparison of sequence coverage of two proteins, ubiquitin and myoglobin, using HCD, ETD, EThcD, and AI-ETD.

FIGS. 23-26 similarly illustrate the sequence coverage of carbonic anhydrase. FIG. 23 shows comparison of sequence coverage between AI-ETD, EThcD, ETD and HCD. FIG. 24 shows exemplary MS spectra using ETD and AI-ETD. AI-ETD resulted in greater sequence coverage and a greater number of unique fragments. FIG. 25 is a graph illustrating the sequence coverage as a function of the precursor charge state. FIG. 26 shows the matched peptide fragments using AI-ETD at a charge state of 30 and HCD at a charge state of 24 (81% sequence coverage was obtained).

FIGS. 27 and 28 show sequence coverage when AI-ETD was performed on enolase. FIG. 27 shows the effects on sequence coverage using shorter reaction times/lower NCE, longer reaction times/higher NCE, and a combination of both. FIG. 28 shows the number of matched fragments and the matched amino acid sequence of enolase using AI-ETD combined with HCD.

FIGS. 29-32 show fragmentation spectra of various glycopeptides, including the peptide backbone and glycan fragmentation, using AI-ETD.

FIGS. 33 and 34 show the number of unique glycosites, glycopeptides, and confidently localized glcyo peptide spectral matches (PSMs) using AI-ETD, ETD and EThcD and that the number of glycosites and glycopeptides that can be confidently characterized by AI-ETD can match and surpass state-of-the-art glycoproteomic studies in considerably less time.

FIG. 35 shows MS/MS success rates of ETD and AI-ETD overlaid onto the glycopeptide precursor m/z distribution from a glycoproteomic experiment (gray histogram). ETD suffers from low success rates at higher m/z, limiting its ability to sequence a large proportion of glycopeptides. AI-ETD significantly boosts success rates, providing an avenue to leverage electron-driven dissociation for the majority of glycopeptide precursors.

FIG. 36 further illustrates that generation of sequence-informative product ions is significantly increased with AI-ETD compared to ETD.

FIG. 37 shows single scan ETD and AI-ETD spectra of the same glycopeptide precursor. Annotated peaks represent peptide and glycan fragments, and the ‘N’ shows the glycosite.

FIG. 38 shows graphs comparing ETD and AI-ETD in large-scale glycoproteomic experiments. a) The total ion current (i.e., signal) harbored in peptide sequence informative product ions was calculated for each glycopeptide spectral match made with ETD and AI-ETD, and the distribution is shown here in box plots. b) The histogram compares peptide backbone sequence coverage achieved for each glycopeptide sequenced with ETD and AI-ETD. c) AI-ETD greatly increases the number of glycopeptides sequenced at higher m/z values. d) AI-ETD improves the number of glycopeptides sequenced at both high and low precursor ion charge states, but makes the greatest impact for triply protonated glycopeptides.

FIG. 39 shows a single scan AI-ETD spectrum of a glycopeptide containing a relatively large high mannose glycan. Annotated peaks represent peptide and glycan fragments, and the highlighted ‘N’ shows the glycosite. The extensive fragmentation of both the peptide backbone from electron-driven dissociation and the glycan backbone from vibrational activation via infrared photo-dissociation show that AI-ETD can access a wealth of information about intact glycopeptides in a single MS/MS event.

FIG. 40 provides a summary of N-glycoproteome characterization with AI-ETD. Identifications made with AI-ETD are shown in the center and right of each (localized and total, respectively) and the number of unique glycopeptides, glycosylated proteins, and unique glycosites are compared with the benchmark for previous global glycoproteome analysis. The relative gain over the previous work is shown in the circles.

FIG. 41 illustrates a previous design attempting to use a laser to enhance ETD; however, this design does not focus the laser into the high-pressure or low-pressure cells of a dual pressure linear ion trap as described herein. Instead, this design requires focusing the laser into a highly specialized multipurpose dissociation cell which is not present in the current invention.

FIG. 42 shows instrument modifications used to perform Activated Ion Electron Transfer Dissociation (AI-ETD). The top panel shows implementation of AI-ETD on the Orbitrap Fusion Lumos device as described in FIG. 1, panel a. The laser is mounted to the back of the instrument chassis and the photon beam is positioned to be concentric to the trapping volume of the linear ion trap. The highlighted components (in grey) will be eliminated with the use of hollow silica waveguides or optical fibers (as seen in the bottom panel), where an optical fiber is used to deliver the light from the laser to the ion trap. Two distinct advantages with the use of optical fibers or hollow core waveguides are (1) that the position of the laser head position is no longer constrained (i.e., the laser can be placed anywhere in the instrument), and (2) the adapter cover with focusing lens can be simply swapped in place of the standard OEM vacuum cover which requires no further alignment.

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

As used herein, the term “mass spectrometer” refers to a device which creates ions from a sample, separates the ions according to mass, and detects the mass and abundance of the ions. Mass spectrometers include multistage mass spectrometers which fragment the mass-separated ions and separate the product ions by mass one or more times. Multistage mass spectrometers include tandem mass spectrometers which fragment the mass-separated ions and separate the product ions by mass once.

As used herein, the term “ion source” refers to a device component which produces ions from a sample. Examples of ion sources include, but are not limited to, electrospray ionization sources, MALDI sources, chemical ionization sources, laser desorption sources, sonic spray sources, photoionization sources, desorption sources, and fast ion bombardment sources.

As used herein, the term “mass analyzer” refers to a device which separates particles, especially ions, according to the mass of the ions. Mass analyzers can also separate ions according to their mass-to-charge ratios. Mass analyzers include secondary mass analyzers which separate product ions according to their mass. As used herein, the term “mass-to-charge ratio” refers to the ratio of the mass of a species to the charge state of a species.

As used herein, the term “ion trap” refers to a device which captures ions in a region of a vacuum system. Ion traps include, but are not limited to, quadrupole ion traps, Fourier transform ion cyclotron resonance ion traps, linear quadruple ion traps, orbitrap ion traps, and quadrupole mass analyzers.

As used herein, the term “ion optic” refers to a device component which assists in the transport and manipulation of charged particles, for example ions, by the application of electric and/or magnetic fields. The electric or magnetic field can be static, alternating, or can contain both static and alternating components. Ion optical device components include, but are not limited to, ion deflectors which deflect ions, ion lenses which focus ions, and multipoles (such as quadruples) which confine ions to a specific space or trajectory. Ion optics can include multipole RF device components which comprise multiple rods having both static and alternating electric and/or magnetic fields.

As used herein, the term “differentially pumped chamber” refers to a vacuum chamber which has regions of different local pressure which are in fluid communication with each other. The regions of different local pressure can be separated by an aperture to allow fluid communication between the regions. Likewise, two or more differentially pumped chambers can be in fluid communication with each other and be separated by apertures to allow fluid communication between the two or more differentially pumped chambers. Differentially pumped chambers can comprise one or more ion optical elements, such as multipoles, lenses, and ion selection devices.

As used herein, the term “collision cell” refers to a chamber of a mass spectrometer which can be isolated to contain one or more neutral gases which interact with a beam of ions where the interaction fragments at least a portion of the ions.

As used herein, the term “fluid communication” refers to elements, such as device elements, which are in fluid contact with each other. A fluid can include, but is not limited to, a gas, a liquid, or a super-critical fluid. Elements which are in fluid contact with each other can pass gasses, for example gasses containing ions, between them.

As used herein, the term “controller” refers to a device component which can be programmed to control a device or system, as is well known in the art. Controllers can, for example, be programmed to control mass spectrometer systems as described herein. Controllers can be programmed, for example, to carry out ion manipulation and sample analysis methods as described herein on systems and devices as described herein.

As used herein, the term “fractionated” or “fractionate” refers to the physical separation of a sample, as is well known in the art. A sample can be fractionated according to physical properties such as mass, length, or affinity for another compound, among others using chromatographic techniques as are well known in the art. Fractionation can occur in a separation stage which acts to fractionate a sample of interest by one or more physical properties, as are well known in the art. Separation stages can employ, among other techniques, liquid and gas chromatographic techniques. Separation stages include, but are not limited to, liquid chromatography separation systems, gas chromatography separation systems, affinity chromatography separation systems, and capillary electrophoresis separation systems.

As used herein, the terms “product ion” refers to an ion which is produced during a fragmentation process of a precursor ion, such as beam-type collision-activated dissociation fragmentations, electron reaction dissociation, and ion reaction dissociation.

As used herein, the term “beam-type collision-activated dissociation” refers to the process of dissociating precursor ions by accelerating the ions to a high kinetic energy and colliding the ions with a background gas, causing dissociation or fragmentation of the precursor ions. In an embodiment, for example, precursor ions are accelerated to a high kinetic energy and collided with a high pressure background gas, for example a pressure greater than 0.01 Torr, in an ion injection pathway or inlet which results in the production of product ions from dissociation or fragmentation of the precursor ions. The background gas can comprise constituents of air or other gas from an ion source, for example helium, nitrogen, oxygen, and/or argon.

As used herein, the term “ion reaction dissociation” refers to a process whereby precursor ions react with another ionic species and the reaction products include product ions produced by fragmentation of the precursor ions. In an embodiment, for example, the reaction is an electron transfer dissociation reaction in which electrons are transferred to the precursor ions from a radical ion species. In an aspect, the radial ion species is an anthracene, azobenzene, or derivative or mixture thereof.

As used herein, the term “electron reaction dissociation” refers to a process whereby precursor ions react with electrons and the reaction products include product ions produced by fragmentation or dissociation of the precursor ions. In an embodiment, for example, the electron reaction dissociation is an electron capture dissociation in which low kinetic energy electrons react with the precursor ions and result in the dissociation of at least a portion of the precursor ions to form product ions. In an embodiment, for example, at least a portion of the product ions retain at least one posttranslational modification of the parent ions, such as phosphorylation and/or O-glycosylation.

Overview

The present invention provides a new approach for outfitting a mass spectrometer with a laser, preferably an infrared (IR) laser, that provides an improved method of ion dissociation through the use of a unique laser beam delivery system. Generally referred to herein as Activated Ion Electron Transfer Dissociation (AI-ETD), the fragmentation method utilizes additional energy from photons to generate extensive fragmentation by interacting with peptides or proteins that are not fully fragmented/separated in the high pressure linear ion trap, thus allowing for increased information during MS/MS.

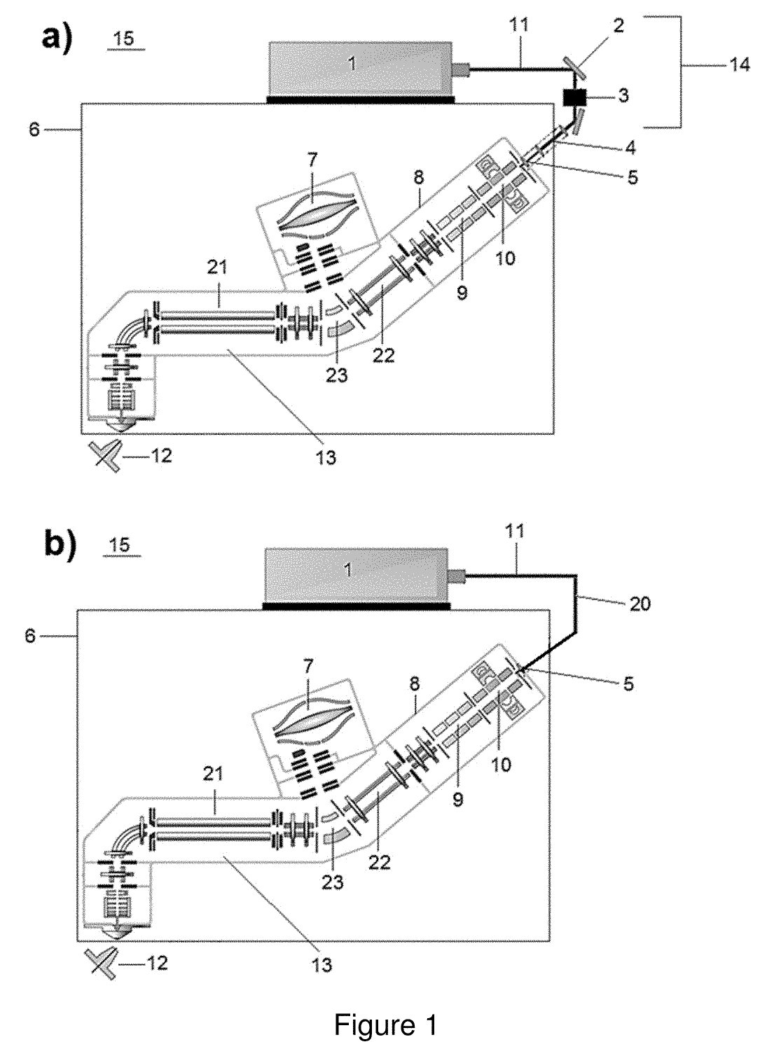

Instrument modifications for AI-ETD in two exemplary embodiments are illustrated in FIG. 1 which shows a modified version of a dual cell linear ion trap on an Orbitrap Fusion Lumos (q-OT-LIT).

FIG. 1, panel (a), is an instrument schematic of a mass spectrometer device (15) having an optical assembly (14), which comprises an infrared laser (1) (in a specific example, a continuous wave 60 W CO₂ laser, λ=10.6 μm), guiding mirrors (2), beam steerer (3), focusing lenses (4), and entrance window (5). Ions are generated from the ion source (12) and are transported through the ion pathway (13) into the dual-pressure linear ion trap (8). The ion pathway (13) in this embodiment comprises a quadrupole mass filter (21), ion-routing multipole (22), and C trap (23). Within the linear ion trap (8), the ions are shuttled between the high-pressure cell (9) and the low-pressure cell (10).

In one embodiment, the photon beam (11) is directed through the entrance window (5) and into the high-pressure cell (9) while the ions are present in the high-pressure cell (9) in order to enhance fragmentation (such as ETD). In a further embodiment, the photon beam (11) is directed through the entrance window (5) and into the low-pressure cell (9) while the ions are present in the low-pressure cell (9) in order to induce additional fragmentation (such as CAD). After fragmentation, the fragmented ions are transported through the ion pathway (13) to the mass analyzer (7).

FIG. 1, panel (b), is an instrument schematic of a similar mass spectrometer device where the photon beam (11) is transmitted from the laser (1) to the entrance window through a hollow silica waveguide (20). This design is beneficial in that the hollow silica waveguide (20) may be simpler to install and provides greater flexibility than the guiding mirrors (2), beam steerer (3), and lenses (4) depicted in FIG. 1, panel (a). Additionally, the photon beam is less exposed to the surrounding area if it is transmitted through the silica waveguide.

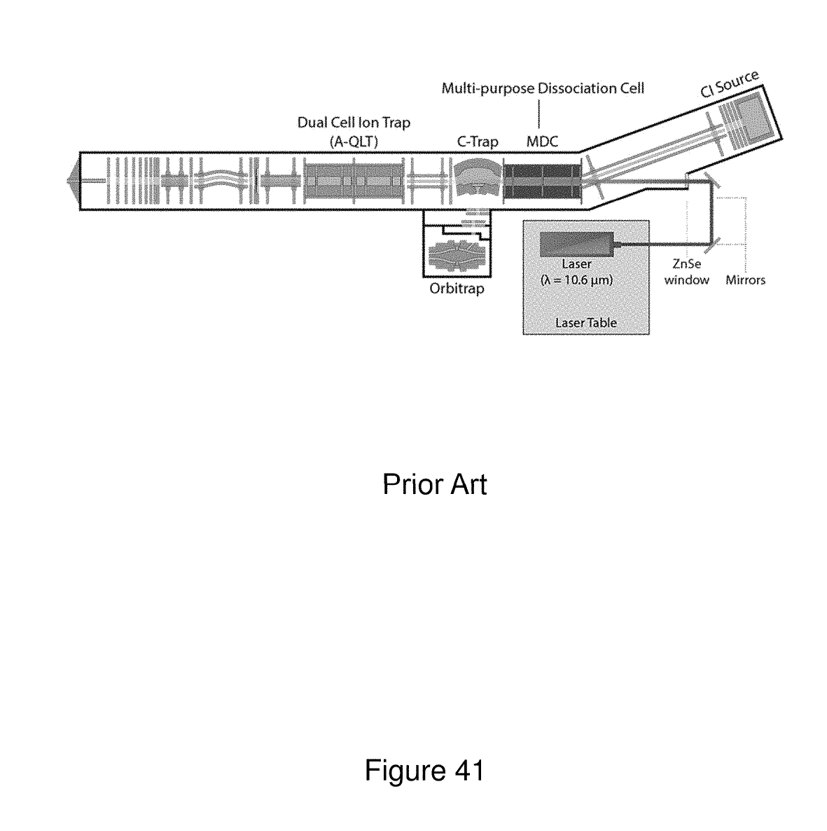

The use of lasers to enhance ETD has been described previously (see Ledvina et al., J. Am. Soc. Mass Spectrom., 2013, 24: 1623-1633 and Rose et al., J. Am. Soc. Mass Spectrom., 2013, 24: 816-827); however, such attempts did not focus the laser into the high-pressure and low-pressure cells of a dual pressure linear ion trap as described herein. Instead, these references utilized a different design which required focusing the laser into a highly specialized multipurpose dissociation cell, which is not part of the present invention (FIG. 41).

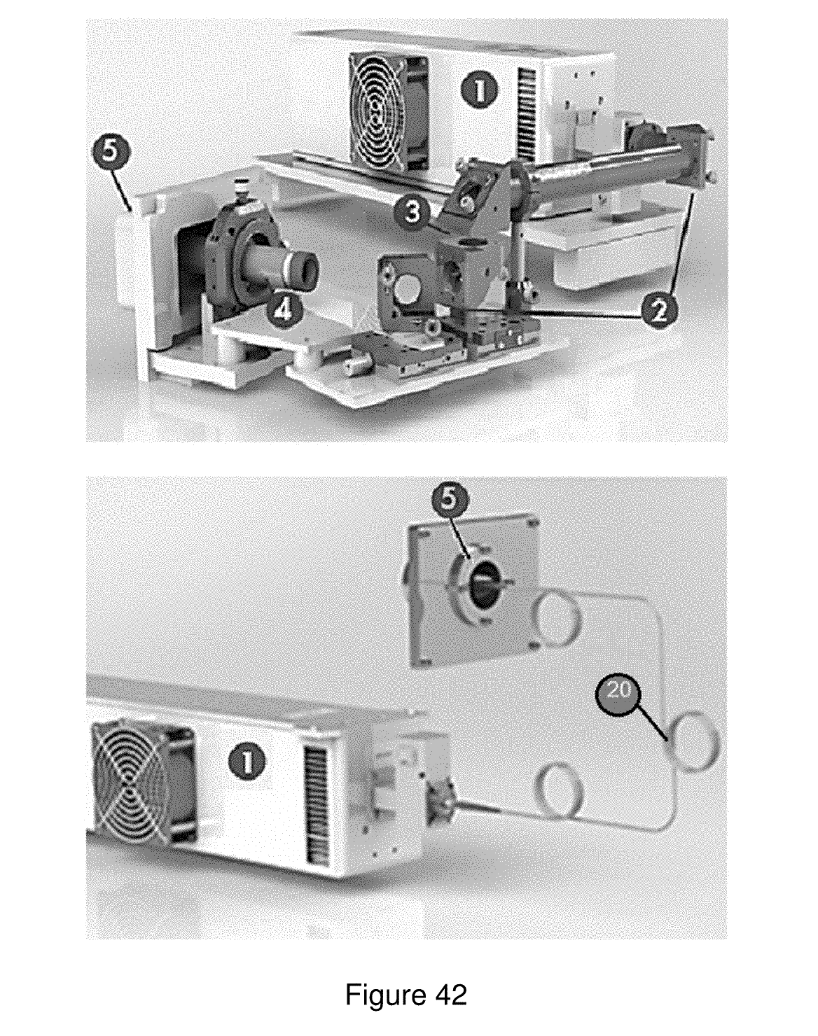

The optical assembly (14) of the present invention can be mounted to the chassis (6) of the mass spectrometer so as to eliminate the need for a separate laser table and to ensure proper alignment of the resulting photon beam (11). All beam guiding and manipulation is done external to the vacuum chambers of the instrument. Depictions of AI-ETD devices are shown in FIG. 42. For example, the top panel the orientation of the laser mounted to the back of the instrument chassis and how the photon beam is positioned to be concentric to the trapping volume of the linear ion trap using various guiding mirrors, beam steerers and lenses. The bottom panel shows a similar device but where an optical fiber or hollow silica waveguide is used to transmit the photon beam into the ion trap.

AI-ETD enables improved characterization of several classes of biomolecules. Comparative analysis shows that the AI-ETD greatly increases fragmentation (almost double improvement) of unmodified peptides, phosphorylated peptides, glycosylated peptides, and intact proteins (both unmodified and modified with various PTMs).

In addition, the present invention provides a method (herein also referred to as “AI-ETD+”), wherein the laser is used to induce CAD in the low pressure ion trap. This implementation is limited to devices having separate ion traps for fragmentation (high pressure, ETD) and mass analysis (low pressure).

### EXAMPLES

### Example 1—Implementation of Activated Ion Electron Transfer Dissociation on a Quadrupole-Orbitrap-Linear Ion Trap Hybrid Mass Spectrometer

Using concurrent IR photo-activation during electron transfer dissociation (ETD) reactions, i.e., activated ion ETD (AI-ETD), significantly increases dissociation efficiency resulting in improved overall performance. This example describes implementation of AI-ETD on a quadrupole-Orbitrap-quadrupole linear ion trap (QLT) hybrid MS system (Orbitrap Fusion Lumos) and demonstrates the substantial benefits it offers for peptide characterization. First, it is shown that AI-ETD can be implemented in a straight-forward manner by fastening the laser and guiding optics to the instrument chassis itself, making alignment with the trapping volume of the QLT simple and robust. The performance of AI-ETD is then characterized using standard peptides in addition to a complex mixture of tryptic peptides using LC-MS/MS, showing not only that AI-ETD can nearly double the identifications achieved with ETD alone, but also that it outperforms the other available supplemental activation methods (ETcaD and EThcD). Finally, a new activation scheme called AI-ETD+ is described that combines AI-ETD in the high pressure cell of the QLT with a short infrared multi-photon dissociation (IRMPD) activation in the low pressure cell. This reaction scheme introduces no addition time to the scan duty cycle but generates MS/MS spectra rich in b/y-type and c/z●-type product ions. The extensive generation of fragment ions in AI-ETD+ substantially increases peptide sequence coverage while also improving peptide identifications over all other ETD methods, making it a valuable new tool for hybrid fragmentation approaches.

A major challenge of AI-ETD is the addition of an IR laser on the mass spectrometer system. Previous implementation of AI-ETD on an Orbitrap system required extensive hardware modifications (Ledvina et al., J. Am. Soc. Mass Spectrom., 2013, 24: 1623-1633; Rose et al., J. Am. Soc. Mass Spectrom., 2013, 24: 816-827; and Riley et al., Anal. Chem., 2015, 87: 7109-7116), but the geometry of the newest generation of Orbitrap MS, i.e., the quadrupole-Orbitrap-quadrupole linear ion trap (q-OT-QLT) hybrid ( ) Senko et al., Anal. Chem., 2013, 85: 11710-11714), or the Orbitrap Fusion Lumos, enables facile implementation of the laser for robust AI-ETD experiments. A straight-forward approach of affixing a continuous wave CO₂ laser to the Lumos system is described below and demonstrates that AI-ETD provides the best performance of all ETD methods for shotgun proteomics. Moreover, this implementation of AI-ETD is used to introduce the new AI-ETD+ activation scheme that takes advantage of both electron-driven fragmentation and infrared multi-photon dissociation (IRMPD) to generate full sequence coverage of the large majority of peptides identified in LC-MS/MS experiments.

Materials and Methods

Mass Spectrometry Instrumentation.

A quadrupole-Orbitrap-quadrupole linear ion trap (q-OT-QLT) hybrid MS system (Orbitrap Fusion Lumos, Thermo Fisher Scientific, San Jose, Calif.) was modified to include Firestar T-100 Synrad 60-W CO₂ continuous wave laser (Mukilteo, Wash.). To facilitate alignment of the laser beam concentric to the central axis of the ion trap a series of 4 broadband metallic mirrors (Borofloat 33, Newport, Irvine, Calif.) were used to steer the beam. The arrangement of the mirrors as illustrated in FIG. 1, panel a, allowed for independent pitch, yaw, horizontal adjustment, and vertical adjustment. The use of kinematic mirror mounts (Thorlabs, Newton, N.J.) provided for easy adjustment. To compensate for beam divergence, the laser beam was collimated before entering the mass spectrometer using two ZnSe bi-convex lens (Thorlabs) of focal lengths 25.4 mm and 100.0 mm, initially separated by 14 cm. Subtle adjustment is made to the initial spacing to obtain proper focus. For laser access to the trapping volume the ion trap vacuum chamber was modified to include a ZnSe window (Thorlabs). Final alignment was obtained by adjusting the optical components to maximize ion fragmentation at a fixed laser power.

Standard Peptide Analysis.

Four synthetic peptides with the sequences SEDYVDIVQGNR, ENDILVLMESER, ELVNDDEDIDWVQTEK, and INQLISETEAVVTNELEDGR were obtained from New England Peptides (Gardner, Mass.) and were resuspended together in a mixture at 5 ppm per peptide in 0.2% formic acid (FA)/49.8% H₂O/50% ACN. The four peptide mixture was infused at a flow rate of 5-7 μL/min using the instrument's syringe pump and an ESI source held at +4.5 kV with respect to ground. Precursor ion AGC target values were set to 50,000, ETD reagent ion AGC target values were set to 300,000, the doubly protonated precursor of each peptide was reacted for 100 ms, and fragment ions were analyzed in the Orbitrap with resolution of 15,000 at 200 m/z. Optimal ETcaD and EThcD normalized collision energies (nce) were determined to be 35 nce and 25 nce, respectively (data not shown). AI-ETD laser power varied from 6-30 Watts (W) as indicated in the text. Spectra were analyzed using in-house software written in C# using the C# Mass Spectrometry Library (CSMSL, https://github <dot>com/dbaileychess/CSMSL) to extract fragment ion matches (within a ±15 ppm tolerance) and their intensities. Intensities were measured for individual spectra and the values reported are the average of 50 spectra. Note, spectral averaging was not conducted, but rather the intensity value for each product ion was averaged across 50 measurements.

LC-MS/MS Analysis of Complex Tryptic Peptide Mixture from Mouse Brain.

A whole mouse brain was homogenized in lysis buffer (8M Urea, 50 mM tris) using a probe sonicator, and protein concentration was determined using a BCA Protein Assay Kit (Thermo Pierce, Rockford, Ill.). Tryptic digestion was performed similarly as described elsewhere (Stefely et al., Nat. Biotechnol., 2016, 34: 1191-1197). Briefly, 100 μg of mouse brain lysate was brought to 90% methanol by volume, and proteins were precipitated by spinning the solution for 5 minutes at 12,000 G. The supernatant was discarded, and the resultant protein pellet was resuspended in 8 M urea, 10 mM tris(2-carboxyethyl)phosphine (TCEP), 40 mM chloroacetamide (CAA) and 100 mM tris (pH=8.0). The total urea concentration was diluted to 1.5 mM urea with 100 mM tris and digested with trypsin (Promega, Madison, Wis.) overnight at room temperature (1:50, enzyme/protein). Peptides were desalted using Strata×columns (Phenomenex Strata-X Polymeric Reversed Phase, 10 mg/mL), which were equilibrated with one column volume of 100% acetonitrile (ACN), followed by 0.1% trifluoroacetic acid (TFA). Acidified peptides were loaded on column, followed by washing with three column volumes of 0.1% TFA. Peptides were eluted using 500 μL 40% ACN 0.1% TFA and 500 μL 80% ACN with 0.1% TFA. Peptide concentration was measured using a quantitative colorimetric peptide assay (Thermo Pierce), and peptides were resuspended in 0.2% FA prior to LC-MS/MS analysis.

A reversed-phase column was packed in-house using 75 μm inner diameter, 360 μm outer diameter bare fused silica capillary. A nanoelectrospray tip was laser pulled (Sutter Instrument Company, Novato, Calif.) and packed with 1.7 μm diameter, 130 Å pore size ethylene bridged hybrid C18 particles (Waters) to a length of 30 cm. The column was installed on a Dionex Ultimate 3000 UPLC system and heated to 60° C. using an in house designed column heater (Hebert et al., Mol. Cell. Proteomics, 2014, 13: 339-347; and Richards et al., Nat. Protoc., 2015, 10: 701-714), and stainless steel ultra-high pressure union formatted for 360 μm outer diameter columns (IDEX). Solvent A was 0.2% FA in H₂O and solvent B was 0.2% FA in 80% ACN/19.8% H₂O. Two micrograms of mouse brain peptides were injected onto the column and gradient elution was performed at 325 nL/min, which increased from 0 to 6% B over 6 min, followed by an increase to 55% at 73 min, a ramp to 100% B at 74 min, and a wash at 100% B for the 6 min. The column was then re-equilibrated at 0% B for 10 min, for a total analysis of 90 minutes. Precursors were ionized using a nanoelectrospray source held at +2 kV compared to ground and the inlet capillary temperature was held at 275° C. Survey scans of peptide precursors were collected from 300-1350 Th with an AGC target of 5,000,000, a maximum injection time of 50 ms, and a resolution of 60,000 at 200 m/z. Monoisotopic precursor selection was enable for peptide isotopic distributions, precursors of z=2-6 were selected for data-dependent MS/MS scans for 2 seconds of cycle time, and dynamic exclusion was set to 10 seconds with a ±10 ppm window set around the precursor.

Calibrated charge dependent ETD parameters were enabled to determine ETD reagent ion AGC and ETD reaction times (Rose et al., J. Am. Soc. Mass Spectrom. 2015, 26:1848-1857), and all MS/MS scans utilized the QLT as the mass analyzer with rapid scan speed enabled. The MS/MS AGC target value was set to 20,000 with a maximum injection time of 20 ms, and precursors were isolated with a 1.2 Th window using the quadrupole. Normalized collision energies of 35 and 25 were set for ETcaD and EThcD experiments, respectively, and AI-ETD laser powers varied from 12-21 W. Tandem mass spectra were searched with the Open Mass Spectrometry Search Algorithm in the COMPASS suite (Geer et al., J. Proteome Res., 2004, 3: 958-964; and Wenger et al., Proteomics, 2011, 11: 1064-1074). Prior to the search, spectra were “cleaned” such that charge-reduced product ions and neutral losses within the window 60 Da below and 5 Da above the charge-reduced peaks were removed in addition to a ±3 Da window around the unreacted precursor (Good et al., J Am Soc Mass Spectrom, 2009, 20: 1435-1440; and Good et al., Proteomics, 2010, 10: 164-167).

A multi-isotope search using four isotopes with a mass tolerance of ±150 ppm was used for precursors, and a monoisotopic mass tolerance of ±0.35 Da was used for product ions analyzed in the ion trap. For all analyses except EThcD and AI-ETD+ experiments, c-, z●-, and y-type product ions were searched, whereas b-type product ions were also included in searches of EThcD and AI-ETD+ data. Oxidation of methionine was specified as a variable modification, while carbamidomethylation of cysteine was a set as a fixed modification. Trypsin specificity with three missed cleavages allowed was used and peptide spectral matches (PSMs) were made against the Uni Prot mouse (mus musculus) database (canonical and isoforms) downloaded on May 12, 2016, which was concatenated with a reversed sequence version of the forward database. Peptides were filtered to a 1% false discovery rate using both e-value and precursor mass accuracy. When pooling spectra from multiple nLC-MS/MS analyses, the false discovery rate was calculated for the aggregate set of data rather than calculating a separate false discovery rate for each run prior to combining results.

Results and Discussion

Instrument Modifications.

The implementation of the CO₂ laser on a q-OT-QLT system required minimal additional hardware while still allowing for simple laser installation that maintained alignment integrity and robust instrument performance. FIG. 1, panel a, illustrates the approach, where the laser and alignment optics were affixed to the instrument chassis itself, removing the need for a laser table and leaving the instrument footprint largely unchanged. FIG. 1, panel a, shows an overhead view of this setup, indicating how the laser head is attached to the back of the instrument and the beam is guided into the dual cell linear ion trap using optic mirrors and beam steerers. Two focusing lenses were also introduced immediately prior to the ZnSe window (25.4 mm in diameter) that was added into the vacuum manifold. The IR photon beam was focused by the first lens to have a waist approximately 1 mm in diameter, and the second lens columnated the beam prior to transmission into the QLT. FIG. 42, top panel, provides an image of this implementation with appropriate components labeled. The tube housing the lenses makes coarse alignment with the QLT straight-forward and the beam steerers allow for simple adjustment of fining tuning of the beam position in the x and y dimensions.

By having all laser components fastened to the instrument itself, the alignment remains robust and largely unaffected by small movements that could drastically affect a setup where instrument and laser table are on separate foundations. Additionally, minimizing hardware reduces the costs and space needed to outfit the system with a laser. The laser was aligned concentric with the QLT for all experiments as described herein using IRMPD fragmentation of background ions as indicators of alignment accuracy. Following the hardware modifications, the Lua instrument control software was modified to trigger the laser to fire when desired using TTL logic and a gate controller. Laser power was controlled using a spare DAC output on the instrument so that power could be adjusted in real time throughout a given experiment. Importantly, the He bath gas pressures used for normal QLT operation remained unchanged for all experiments.

AI-Etd Performance.

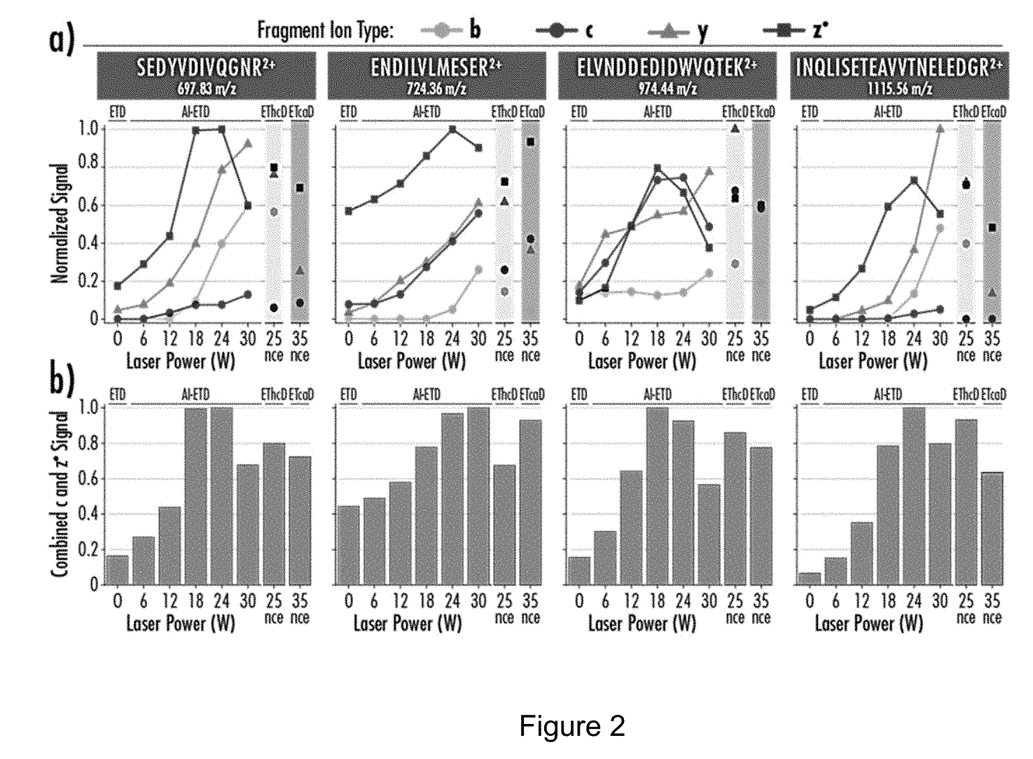

The laser-outfitted q-OT-QLT system was first assessed by dissociating four synthetic tryptic peptides with AI-ETD at various laser powers. For all AI-ETD experiments, the ETD reaction was carried out as normal in the high pressure cell while trapping volume was irradiated only for the duration of the ion-ion reaction with no pre- or post-activation. In this current implementation, laser powers of 12-24 W generally produced the best AI-ETD fragmentation (FIG. 2), which is significantly lower than laser powers need in previous AI-ETD experiments on a linear ion trap-Orbitrap hybrid MS system (Orbitrap Elite) where the pressure in the reaction cell also had to be lowered to achieve optimal AI-ETD fragmentation (Ledvina et al., J. Am. Soc. Mass Spectrom., 2013, 24: 1623-1633; Riley et al., Anal. Chem., 2015, 87: 7109-7116; Riley et al., Mol. Cell. Proteomics, 2015, 14: 2644-2660; and Riley et al., J. Proteome Res., 2016, 15: 2768-2776). The much shorter laser path and use of focusing optics in this implementation account for this difference, allowing use of lower powered lasers.

FIG. 2, panel a, displays the intensity of c-, z●-, b-, and y-type product ions produced by ETD and AI-ETD at varying laser powers (6-30 W) for the doubly protonated precursor of each of the four peptides. Fragments generated by EThcD and ETcaD are also plotted for comparison. For each precursor, intensities of individual fragments are summed for a given fragment ion type, and the intensities of each fragment type are normalized to the greatest value seen for that precursor, allowing for easy comparison across each peptide. FIG. 2, panel b, presents the normalized signal (again normalized to the greatest value for each precursor) seen in the c- and z●-type fragment ion channels only, i.e., those specific to ETD fragmentation. In both cases, AI-ETD clearly increases fragment ion production over ETD as laser power increases, although over fragmentation can occur. For each peptide, y-type ion generation increases with laser power, but this is not necessarily indication of collisional fragmentation. ETD is known to produce y-type ions in addition to the canonical c- and z●-type fragments (Chalkley et al., Anal. Chem., 2010, 82: 579-584), so the increase of all three fragment types concomitantly is indicative of improved ETD-like fragmentation. However, increasing b-type ion signal, in addition to degradation of c- and z●-type products, at higher laser powers indicates the onset of IRMPD-like fragmentation, which is considered over fragmentation in AI-ETD experiments.

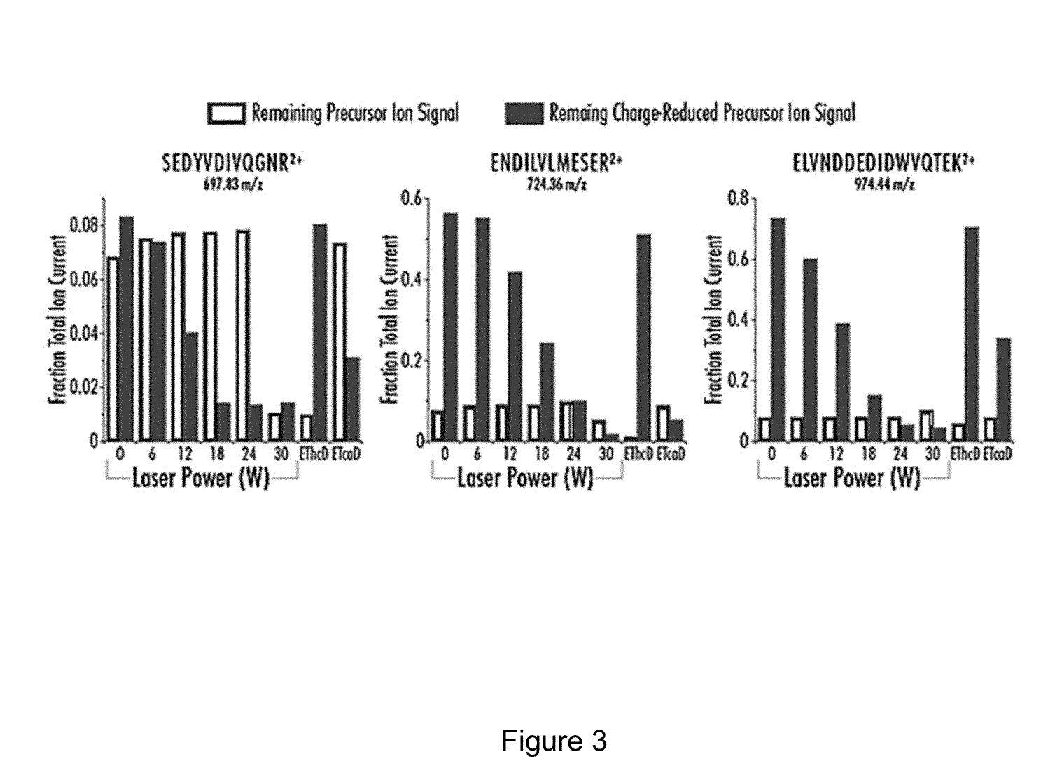

In general, AI-ETD at 18-24 W generated more fragment ion signal than ETD, ETcaD, and EThcD for all four fragment ion types, and AI-ETD always produced the maximum signal for c- and z●-type ions. FIG. 3 further illustrates the increased conversion efficiency of AI-ETD, displaying the amount of remaining precursor and charge-reduced precursor present in spectra after each activation method. This data corresponds to the product ion signals displayed in FIG. 2. As expected, the percent signal explained by the charge-reduced precursor decreased as AI-ETD laser power increased while the signal in the precursor remained fairly constant, indicating that the increase in product ion signal comes from mitigation of ETnoD products that contribute to charge-reduced precursor ion signal. Interestingly, the precursor signal in EThcD fragmentation was significantly reduced while the charge-reduced precursor signal remained similar to ETD. This suggests that rather than fully activate ETnoD products, EThcD generates a significant proportion of additional product ion signal from fragmentation of the remaining precursor, which follows logically as the more highly charged precursor feels more force during the HCD supplemental activation. Note, this is for doubly protonated precursors of the indicated standard peptides and peptide INQLISETEAVVTNELEDGR is excluded because its charge reduced precursor signal is above the 2000 Th limit used for the mass range of these analyses.

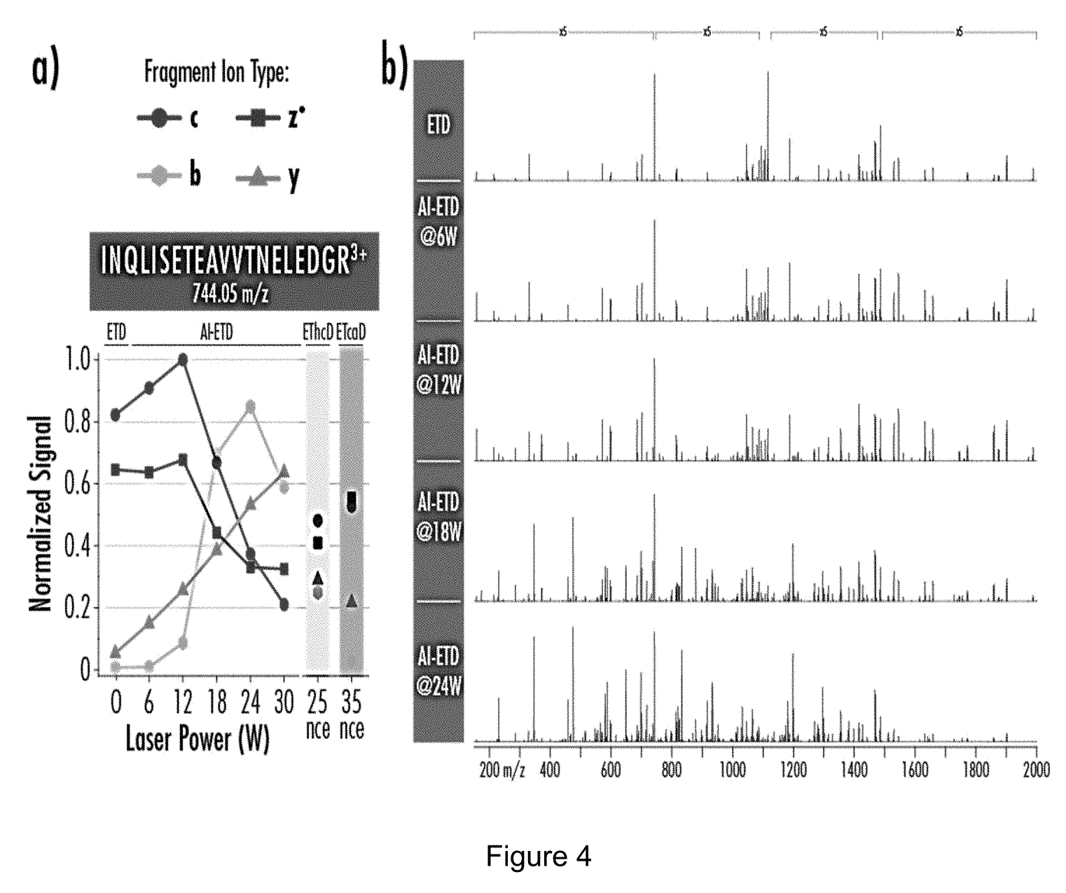

AI-ETD also provided similar improved fragment ion generation for higher charged precursors (z=+3 of INQLISETEAVVTNELEDGR), albeit at lower laser powers (12-18 W) for optimal activation, whereas EThcD and ETcaD both decreased total product ion signal compared to ETD (FIG. 4). Matched fragment ions are available in Table 1, which denotes which product ions appear in which conditions.

TABLE 1
Table 1. Product ion m/z, fragment type and number, and charge are given for
matched fragments from the z = +3 precursor of INQLISETEAVVTNELEDGR (spectra
shown in Supplemental FIG. 2). ‘X’ denotes in which conditions a given product ion
was observed for ETD and AI-ETD at 6, 12, 18, and 24 W laser powers.

| Product Ion | Ion | | | | AIETD | AIETD | AIETD | |
|---|---|---|---|---|---|---|---|---|
| m/z | Type | # | Charge | ETD | 6 W | 12 W | 18 W | AIETD 24 W |
| 582.36 | b | 5 | 1 | | | | X | X |
| 1028.52 | b | 9 | 1 | | | | X | X |
| 1099.55 | b | 10 | 1 | | | X | X | X |
| 599.82 | b | 11 | 2 | | | | X | X |
| 1198.63 | b | 11 | 1 | | | X | X | X |
| 649.85 | b | 12 | 2 | | | | X | X |
| 1297.69 | b | 12 | 1 | | | | X | X |
| 699.98 | b | 13 | 2 | | | | X | X |
| 1398.70 | b | 13 | 1 | | | X | X | X |
| 821.41 | b | 15 | 2 | | | | X | X |
| 877.96 | b | 16 | 2 | | | X | X | X |
| 942.47 | b | 17 | 2 | | | X | X | X |
| 1998.99 | b | 18 | 1 | | | | X | |
| 1029.51 | b | 19 | 2 | | | | X | X |
| 245.16 | c | 2 | 1 | | | X | X | |
| 373.22 | c | 3 | 1 | | X | X | X | |
| 486.30 | c | 4 | 1 | | | X | X | |
| 599.39 | c | 5 | 1 | X | X | X | X | X |
| 686.41 | c | 6 | 1 | X | X | X | X | X |
| 815.46 | c | 7 | 1 | X | X | X | X | X |
| 916.51 | c | 8 | 1 | X | X | X | X | X |
| 1045.53 | c | 9 | 1 | X | X | X | X | X |
| 1215.60 | c | 11 | 1 | X | X | X | X | X |
| 1415.70 | c | 13 | 1 | X | X | X | X | X |
| 1529.80 | c | 14 | 1 | X | X | X | X | X |
| 1659.84 | c | 15 | 1 | X | X | X | X | X |
| 1771.94 | c | 16 | 1 | X | X | X | X | X |
| 1900.98 | c | 17 | 1 | X | X | X | X | X |
| 175.12 | y | 1 | 1 | | | X | X | |
| 232.14 | y | 2 | 1 | | X | X | X | X |
| 347.17 | y | 3 | 1 | | | X | X | X |
| 476.21 | y | 4 | 1 | | | | X | X |
| 589.29 | y | 5 | 1 | | | X | X | X |
| 718.34 | y | 6 | 1 | | X | X | X | X |
| 832.38 | y | 7 | 1 | | X | X | X | X |
| 933.42 | y | 8 | 1 | | | X | X | X |
| 516.75 | y | 9 | 2 | | | | X | X |
| 1032.49 | y | 9 | 1 | X | X | X | X | X |
| 1131.56 | y | 10 | 1 | X | X | X | X | X |
| 1202.60 | y | 11 | 1 | | | X | X | X |
| 1331.60 | y | 12 | 1 | X | X | X | | |
| 1432.68 | y | 13 | 1 | X | X | X | X | |
| 1561.73 | y | 14 | 1 | | X | X | X | |
| 824.88 | y | 15 | 2 | | | | X | X |
| 1648.76 | y | 15 | 1 | X | X | X | X | X |
| 1761.84 | y | 16 | 1 | | X | X | X | |
| 1874.93 | y | 17 | 1 | X | X | X | X | |
| 1058.05 | y | 19 | 2 | | | X | X | |
| 159.10 | zdot | 1 | 1 | X | X | X | X | |
| 216.12 | zdot | 2 | 1 | X | X | X | X | |
| 331.15 | zdot | 3 | 1 | X | X | X | X | |
| 460.19 | zdot | 4 | 1 | X | X | X | X | |
| 573.28 | zdot | 5 | 1 | X | X | X | X | X |
| 702.32 | zdot | 6 | 1 | X | X | X | X | X |
| 816.48 | zdot | 7 | 1 | X | X | X | X | X |
| 917.49 | zdot | 8 | 1 | X | X | X | X | X |
| 1016.48 | zdot | 9 | 1 | X | X | X | X | X |
| 1115.54 | zdot | 10 | 1 | X | X | X | X | X |
| 1186.58 | zdot | 11 | 1 | X | X | X | X | X |
| 1315.60 | zdot | 12 | 1 | X | X | X | X | X |
| 1416.60 | zdot | 13 | 1 | X | X | X | X | X |
| 1545.71 | zdot | 14 | 1 | X | X | X | X | X |
| 1632.74 | zdot | 15 | 1 | X | X | X | X | X |
| 1745.83 | zdot | 16 | 1 | | X | X | X | |
| 1858.92 | zdot | 17 | 1 | X | X | X | X | X |
| 1986.97 | zdot | 18 | 1 | X | X | X | X | |

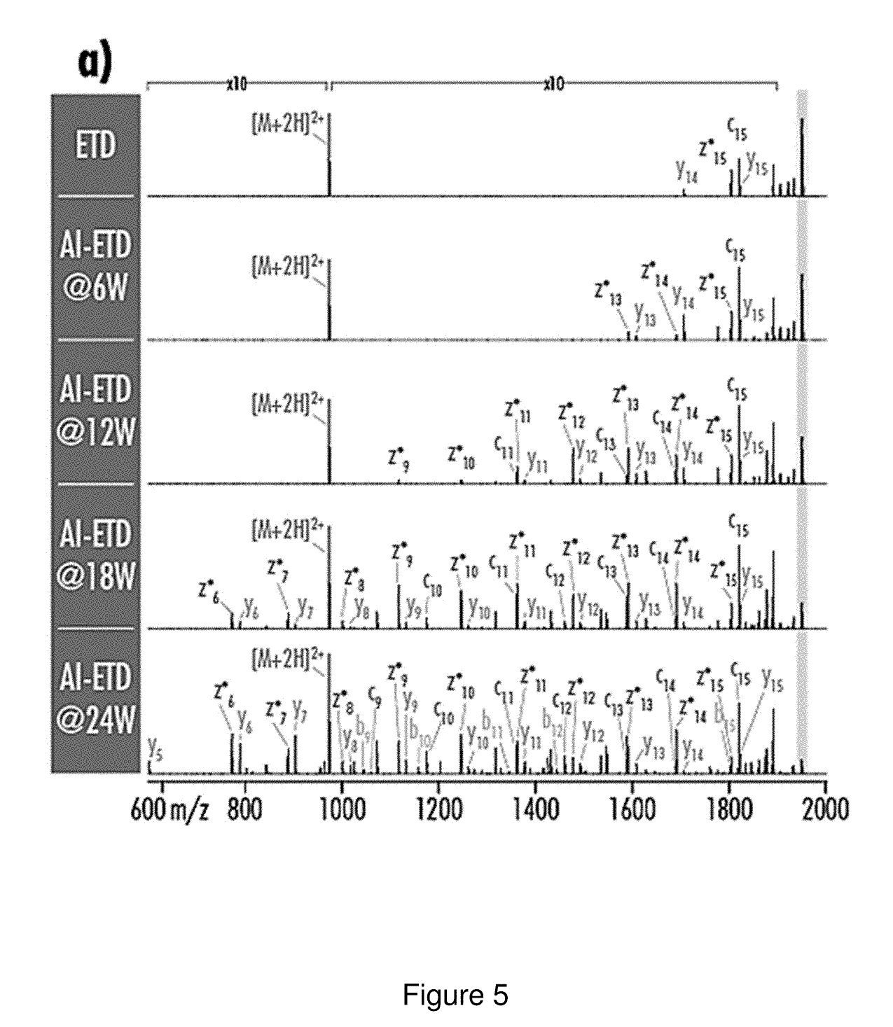

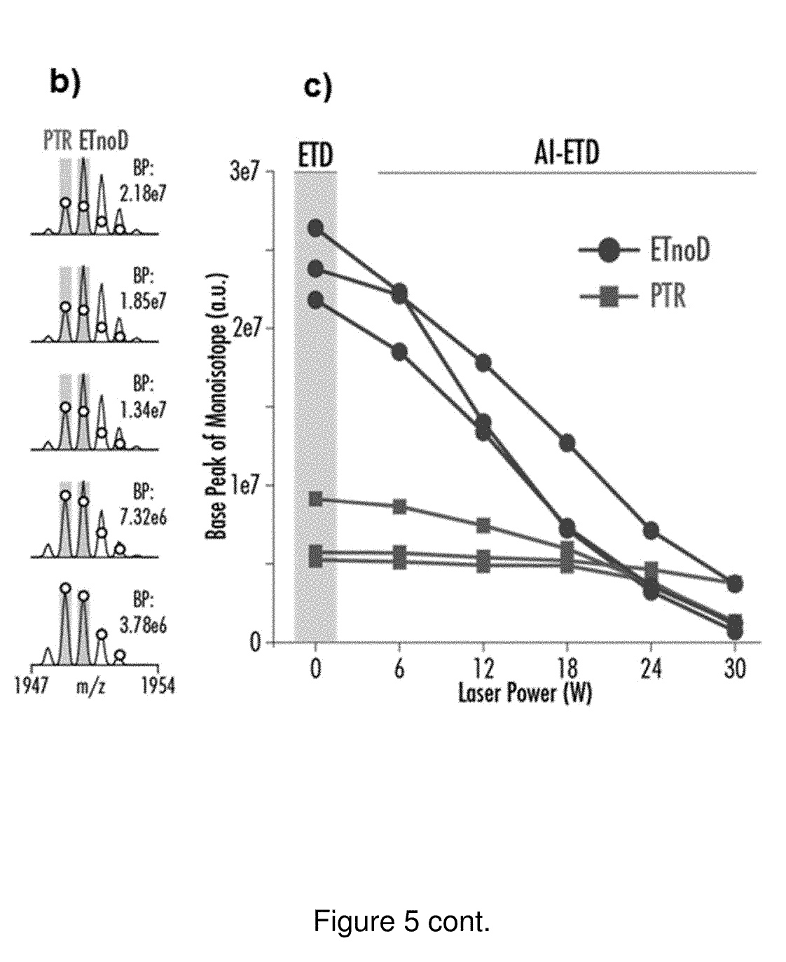

Increased fragment ion yield with AI-ETD is highlighted in FIG. 5, panel a, which shows annotated spectra for ETD and AI-ETD at increasing laser powers for the doubly protonated precursor ion of ENVNDDEDIDWVQTEK. The presence of c-, z●-, and y-type in AI-ETD up through 18 W of power is clear, and the presence of b-type ions in the spectrum AI-ETD at 24 W marks the onset of some degree of IRMPD fragmentation as well. This improvement in fragment ion generation is coming directly from unfolding of precursor ions, which mitigates the formation of non-sequence informative ETnoD products, as demonstrated by FIG. 5, panel b. Here the isotopic envelopes of the charge-reduce precursor ions are shown for each of the spectra in part a (highlighted in gray in FIG. 2, panel a). Monoisotopic m/z peaks of the proton transfer (PTR) and ETnoD products are highlighted. Proton transfer and electron transfer are competing pathways in ETD reactions that vary based on reagent anion used, and a minimal degree of PTR is known to happen with fluoranthene, the reagent used in these experiments (Coon et al., Int. J. Mass Spectrom., 2004, 236: 33-42; Gunawardena et al., J. Am. Chem. Soc., 2005, 127: 12627-12639; and Lermyte et al., Int. J. Mass Spectrom., 2015, 390: 146-154). If ETnoD were not occurring at all and PTR was the only contributing factor to the charge-reduced precursor products, the isotope distribution should match the theoretical isotope distribution shown by open white circles. Clearly, ETnoD products are a major contribution to the charge-reduced precursor products in ETD, but ETnoD products are diminished and ultimately are eliminated as laser power increases with AI-ETD. Note, the isotope distributions are not plotted on the same intensity scale, but the base peak height is given for each. FIG. 5, panel c, illustrates this concept for other standard peptides, plotting the intensity of the ETnoD and PTR monoisotopic peaks as a function of laser power. PTR products are largely unchanged by AI-ETD fragmentation (which is expected because PTR is largely a function of reagent), but ETnoD products are greatly reduced at higher AI-ETD laser powers.

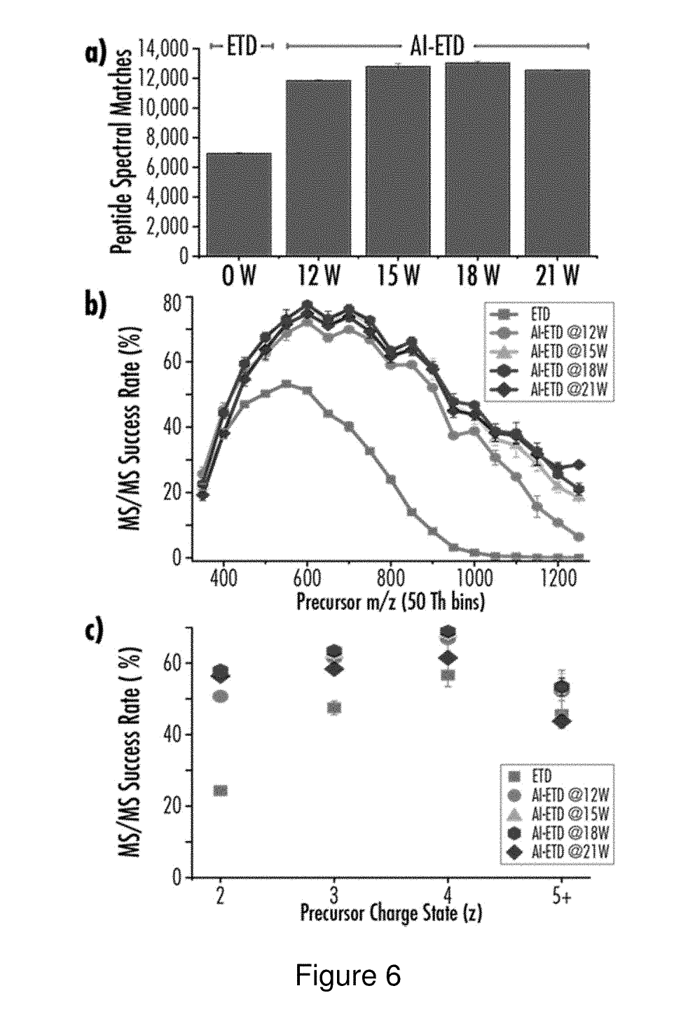

With successful implementation of AI-ETD evident from the synthetic peptide studies, the performance of AI-ETD was examined for shotgun proteomic experiments with 90-minute LC-MS/MS analyses of tryptic peptides from mouse brain lysate. FIG. 6 summarizes the results of these experiments, showing how AI-ETD at various laser powers compares to ETD. AI-ETD (18 W) nearly doubled the number of peptide spectral matches (PSMs) as compared to ETD (panel a). AI-ETD also improves MS/MS success rate, plotted both as a function of precursor m/z (panel b) and precursor charge state (z) (panel c). MS/MS success rate is defined as the number of PSMs successfully sequenced divided by the number of MS/MS scans acquired for a given m/z bin or charge state. The significant decline in success rate with ETD for precursors larger that ˜600 Th and the low success rates for z=+2 precursors exemplify of ETD's low efficiency with low charge density precursors due to ETnoD. AI-ETD improves success rates across the entire m/z range and for all precursor charge states, with the most drastic improvements coming for doubly protonated precursors and high m/z ions. Notably, AI-ETD improves MS/MS success rate for all precursor ion charge states, even the z 3 precursor ions, where ETD is known to perform well (Good et al., Mol. Cell. Proteomics, 2007, 6: 1942-1951; and Swaney et al., Nat. Methods, 2008, 5: 959-964). Comparing between AI-ETD laser powers, the lowest power of 12 W does not improve performance as substantially, but the differences between 15 W, 18 W, and 21 W are less significant, with 18 W providing the best overall results.

Introducing AI-ETD+.

The main goal of AI-ETD is to augment ETD fragmentation; the onset of substantial collisional dissociation products is considered undesirable. Hybrid fragmentation methods, on the other hand, such as EThcD and ETUVPD (i.e., using ultraviolet photo-dissociation to activate ETD reaction products), seek to combine multiple types of fragmentation to generate extensive ion series corresponding to multiple dissociation pathways (Madsen et al., Chemistry 2012, 18, 5374-5383; Cannon et al., Anal. Chem., 2014, 86: 10970-10977; and Shaffer et al., J. Mass Spectrom., 2015, 50: 470-475). This approach has several benefits, including extensive peptide backbone sequence coverage and localization of post-translational modifications (PTMs) that have been demonstrated with EThcD (Frese et al., Anal. Chem., 2012, 84: 9668-9673; Frese et al., J. Proteome Res., 2013, 12: 1520-1525; Mommen et al., Proc. Natl. Acad. Sci. U.S.A., 2014, 111: 4507-4512; Riley et al., Anal. Chem. 2016, 88, 74-94). Challenges with EThcD and similar hybrid activation methods, however, include both additional time needed to perform both dissociation methods, and loss of sensitivity that can occur during the HCD process (i.e., some degree of product ion signal is lost during HCD fragmentation).

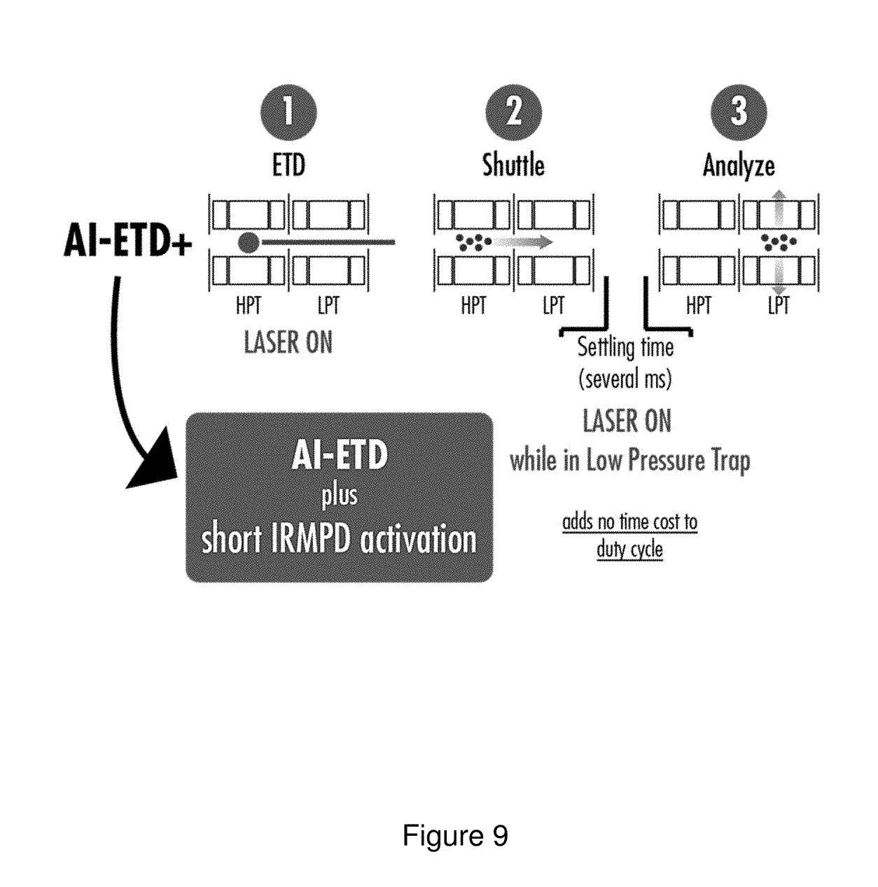

A new hybrid fragmentation approach was devised called AI-ETD+ that combines AI-ETD in the high pressure cell of the dual cell QLT with a short IRMPD activation in the low pressure cell prior to fragment ion mass analysis that occurs in the low pressure trap (see FIG. 9 for a sample scan event). The higher bath gas pressures of the high pressure cell (˜5 mTorr) are suitable for ETD reactions, but they generally prevent dissociation via IRMPD due to collisional cooling (Second et al., Anal. Chem., 2009, 81: 7757-7765; and Gardner et al., Anal. Chem., 2009, 81: 8109-118). The low pressure cell, however, is used for improved resolution and sensitivity during mass analysis (Second et al., Anal. Chem., 2009, 81: 7757-7765) and has low enough pressures (˜0.3 mTorr) to perform IRMPD (Gardner et al., Anal. Chem., 2009, 81: 8109-118; and Ledvina et al., Anal. Chem., 2012, 84: 4513-4519), making the shuttling of ions into the low pressure cell standard in QLT operation and presenting an ideal time to perform IRMPD with no additional steps needed in the scan sequence. IRMPD is known to produce b- and y-type ions similar to other collisional activation methods (Brodbelt et al., Chem. Soc. Rev., 2014, 43: 2757-2783), providing complementary dissociation to AI-ETD. In this scheme, all AI-ETD products are irradiated in the low pressure cell, i.e., no other mass selection occurs prior to the photo-activation, which is similar to EThcD and allows for further fragmentation of the unreacted precursor, a dominant product in all ETD reactions.

Importantly, AI-ETD+ differs from other hybrid fragmentation techniques in that it does not add any additional time to the scan sequence. The IR photo-activation occurs immediately after the transfer of ions into the low pressure trap for a duration of ˜4 ms. This time is typically utilized as a ion cooling time prior to mass analysis in the low pressure cell, but it is more than sufficient to create generate IRMPD products with the reduced bath gas pressure of the low pressure cell.

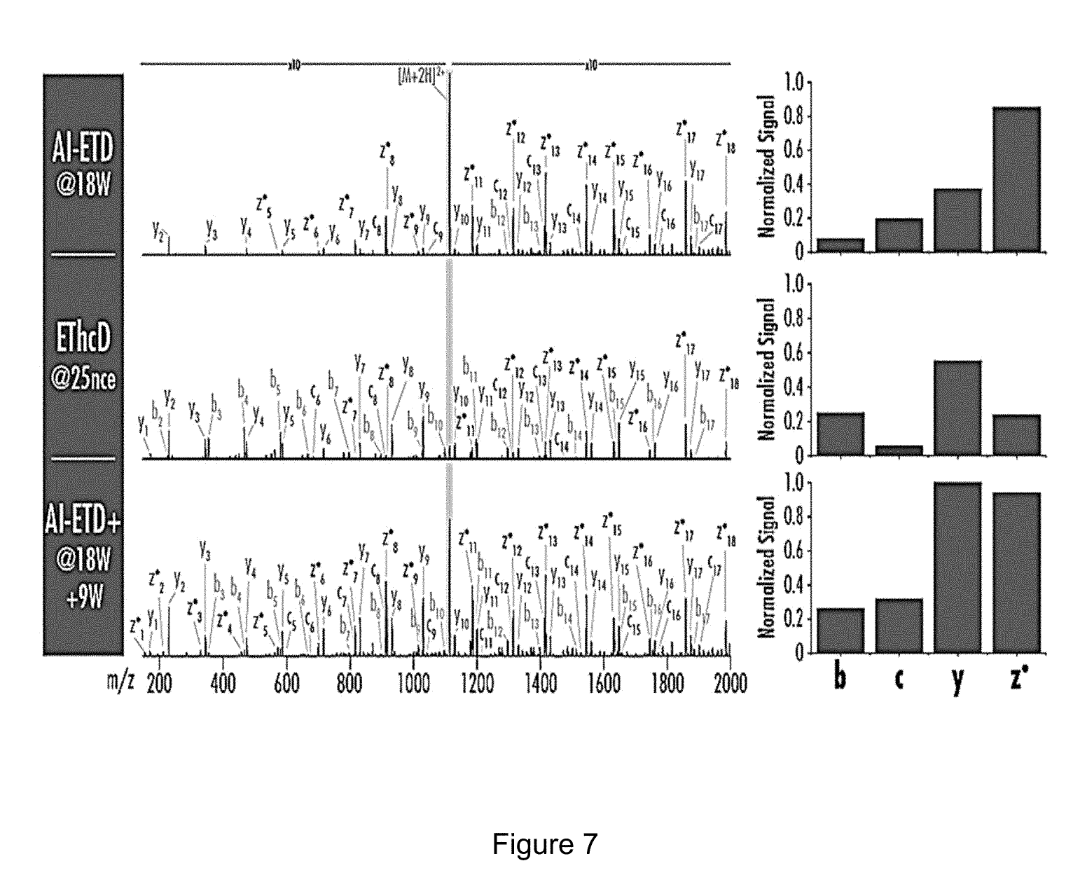

FIG. 7 shows AI-ETD, EThcD, and AI-ETD+ spectra of the doubly protonated precursor of the synthetic peptide INQLISETEAVVTNELEDGR. Fragmentation is seen for each method, and the signal in b-, c-, y, and z●-type ions is shown in bar graphs to the right. This signal is normalized to the most intense signal from all three conditions, which was y-type ion signal in AI-ETD+. The EThcD spectrum has more b- and y-type fragment ions than the AI-ETD spectrum, which is expected, but c- and z●-type products are somewhat diminished—apparent in both the spectra and the bar graphs. AI-ETD+ maintains c- and z●-type product ion signal, with more fragments being generated than in either AI-ETD or EThcD, and it also produces substantial b- and y-type ion series with greater signal than EThcD. Less unreacted precursor is present in the EThcD spectrum, but AI-ETD+ provides more signal in product ions channels. All three spectra are on the same intensity scale, which further highlights the lower overall signal detected in EThcD spectra compared to AI-ETD and AI-ETD+ fragmentation. Different combinations of laser powers were tested for AI-ETD+, with the best combination being 18 W for AI-ETD, followed by 9 W for the IMRPD activation in the low pressure cell. Power output from the laser is easily modulated in the instrument code on this timescale.

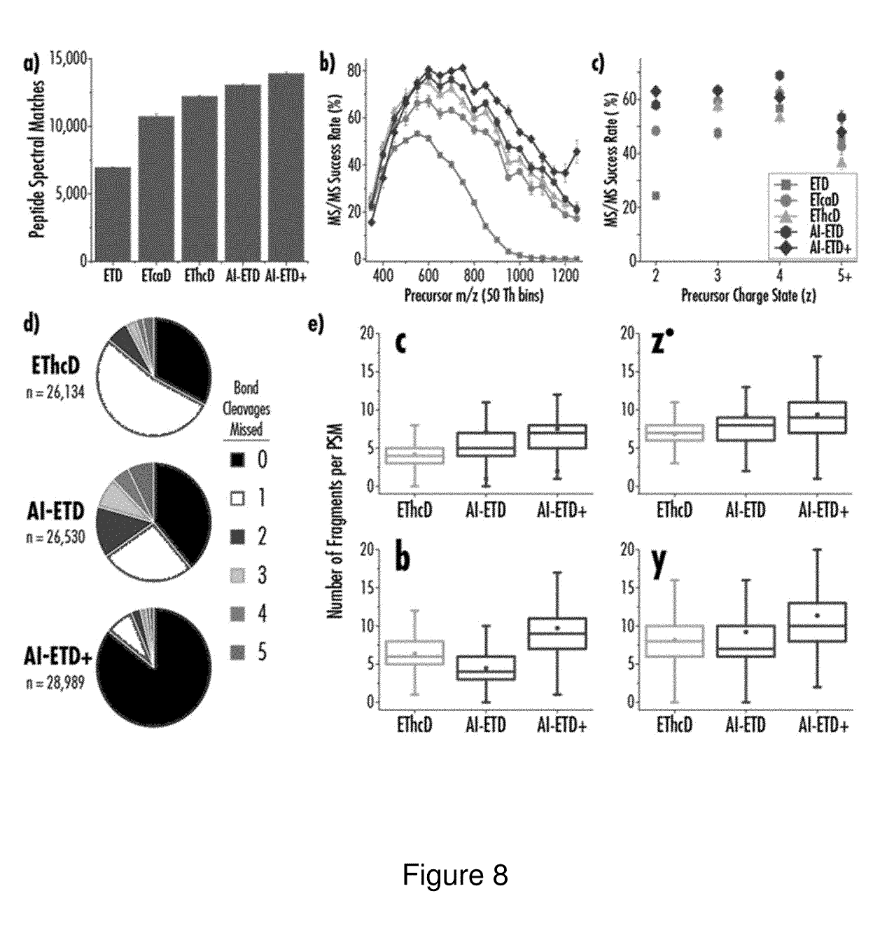

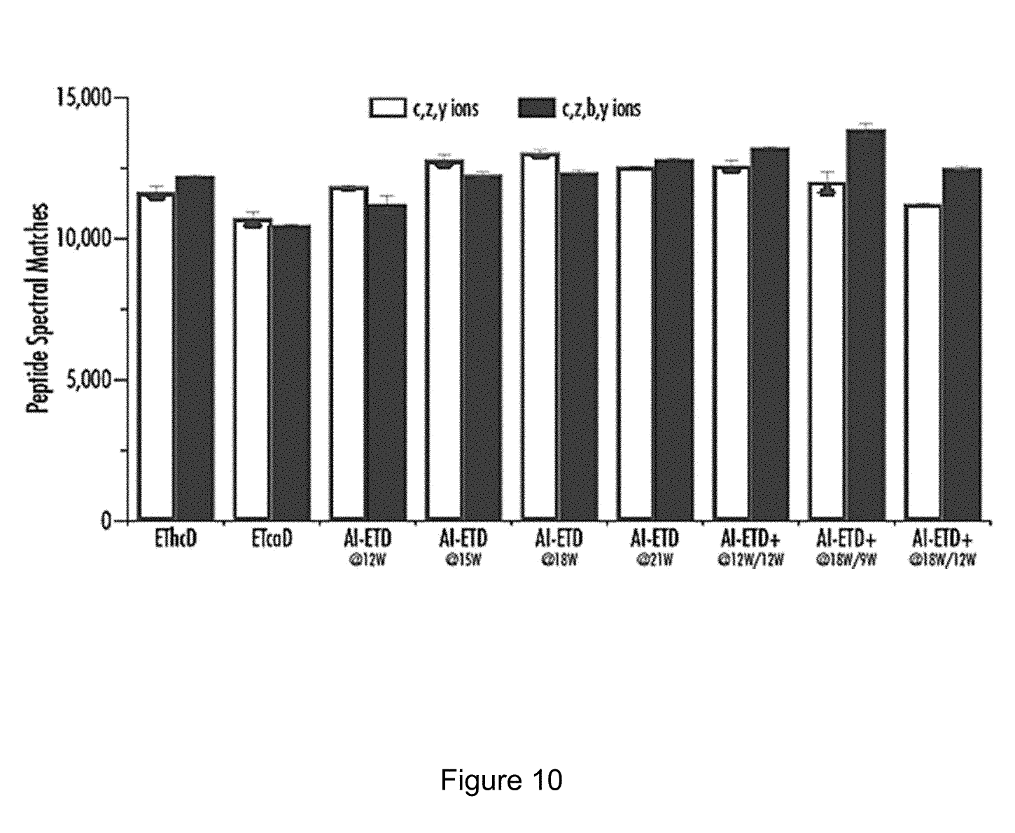

The benefits of AI-ETD+ from this test case translate to LC-MS/MS experiments, as well, making it the optimal supplemental activation technique for ETD. FIG. 8 compares ETD, ETcaD, EThcD, AI-ETD, and AI-ETD+ fragmentation for 90-minute analyses of tryptic peptides from mouse brain lysate. AI-ETD in this comparison uses a laser power of 18 W, AI-ETD+ is shown using a combination of 18 W and 9 W for respective activation stages, and the ETD and AI-ETD data correspond to the analyses shown in FIG. 6. Table 2 below provides the average and standard deviation of MS/MS scans, PSMs, unique peptides, and overall MS/MS success rate for all analyses, including multiple laser powers and laser power combinations for AI-ETD and AI-ETD+, respectively. EThcD and AI-ETD+ data were searched using c-, z●-, b-, and y-type fragments (using all four gave the best performance) while all other analyses used only c-, z●-, and y-type products for searching (FIG. 10).

TABLE 2
Table 2. Average values and standard deviations are shown for several different
ETD analyses with and without supplemental activation. Averages come from three
technical replicate injections. MS/MS success rate, reported as a percentage, is defined
as the number of peptide spectral matches identified divided by the number of MS/MS
scans. An asterisk (*) indicates data was searched using c-, z•-, b-, and y-type
fragments while all other analyses used only c-, z•-, and y-type products for searching.

| | MS/MS | Peptide | Unique | MS/MS |
|---|---|---|---|---|
| | Scans | Spectral Matches | Peptides | Success Rate |
| ETD | 21,971 ± 74 | 6,935 ± 54 | 5,380 ± 93 | 31.56% ± 0.35 |
| ETcaD | 20,591 ± 465 | 10,703 ± 243 | 7,927 ± 233 | 51.99% ± 1.26 |
| *EThcD | 20,954 ± 185 | 12,195 ± 85 | 8,973 ± 79 | 58.19% ± 0.20 |
| Al-ETD | 21,849 ± 218 | 11,845 ± 52 | 8,636 ± 161 | 54.22% ± 0.58 |
| @12 W | | | | |
| Al-ETD | 21,719 ± 471 | 12,793 ± 189 | 9,164 ± 116 | 58.91% ± 0.42 |
| @15 W | | | | |
| Al-ETD | 21,744 ± 350 | 13,037 ± 115 | 9,302 ± 87 | 59.96% ± 0.58 |
| @18 W | | | | |
| Al-ETD | 21,858 ± 351 | 12,543 ± 20 | 9,034 ± 79 | 57.40% ± 0.97 |
| @21 W | | | | |
| *Al-ETD+ | 21,859 ± 449 | 13,220 ± 144 | 9,336 ± 31 | 60.49% ± 0.96 |
| @12 W + 12 W | | | | |
| *Al-ETD+ | 21,931 ± 321 | 13,864 ± 136 | 9,372 ± 209 | 63.22% ± 0.85 |
| @18 W + 9 W | | | | |
| *Al-ETD+ | 21,931 ± 493 | 12,489 ± 202 | 8,877 ± 66 | 57.95% ± 0.34 |
| @18 W + 12 W | | | | |

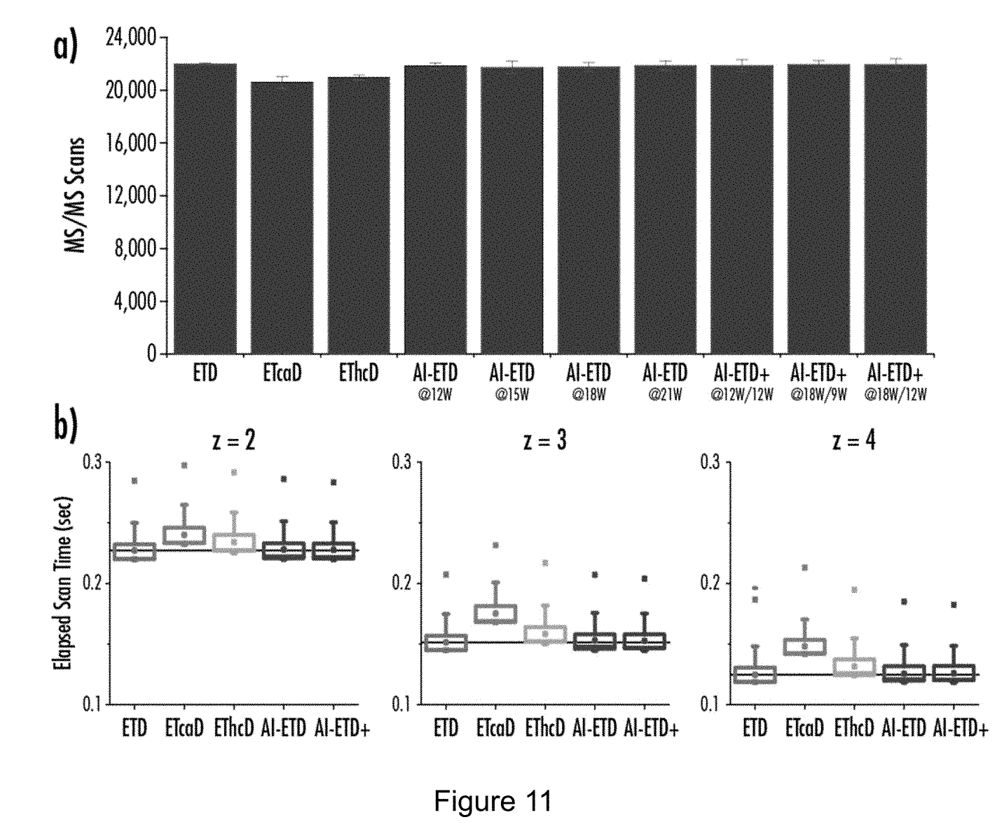

AI-ETD+ provides the greatest number PSMs (FIG. 8, panel a) and generally boosts MS/MS success rate the most across the m/z range and for lower charge state precursors (z=2 and 3). AI-ETD+ was slightly outperformed by AI-ETD and EThcD for precursors with m/z<500 Th (FIG. 8, panel b) and standard AI-ETD is clearly better for higher charged precursors (z≥4) (FIG. 8, panel c), which are factors to keep in mind when designing future experiments. Regardless, AI-ETD or AI-ETD+ provided the best results, and AI-ETD+ doubled the overall MS/MS success rate of ETD (63.22% vs. 31.56%), while AI-ETD and EThcD nearly doubled ETD's success rate (59.96% and 58.19%, respectively). It is worth noting that even with similar success rates, AI-ETD noticeably increased PSM identifications over EThcD. This is likely because more MS/MS scans were acquired in AI-ETD analyses. AI-ETD does not slow down scan acquisition relative to standard ETD due to its concurrent activation regime, whereas EThcD requires additional time to perform the collisional activation step (FIG. 11). Most interestingly, AI-ETD+ also maintained expected numbers of MS/MS scans and did not increase scan times because it utilizes time already built into the scan sequence to perform its additional IRMPD activation (FIG. 11)

Beyond identification metrics, the quality of fragmentation for EThcD, AI-ETD, and AI-ETD+ was also assessed. In FIG. 8, panel d, the extent of peptide backbone sequence coverage is examined by counting the number of bond cleavages missed, i.e., bonds where no fragment ion of any type was observed. If zero bond cleavages are missed, the peptide had 100% sequence coverage, or every bond could be explained by an observable fragment ion. For this comparison, PSMs for all three technical replicate injections were pooled for each fragmentation method, and only z=+2 precursors were considered since they are the majority of precursors selected and are the case were these supplemental activation methods provide the most benefit over ETD. The total numbers of PSMs (n) are provided for each fragmentation method. AI-ETD+ performs significantly better than EThcD and AI-ETD, providing 100% sequence coverage (zero bond cleavages missed) for the large majority (˜85%) of PSMs identified, with another 10% having only one cleavage missed. AI-ETD sequences more PSMs with zero bond cleavages missed than EThcD, but EThcD outperforms AI-ETD when considering PSMs with zero or one missed bond cleavage. AI-ETD+ also generates the highest number of fragment ions per PSM, which is broken down by count of each of the four fragment in types in FIG. 8, panel e. Interestingly, AI-ETD+ produces more b- and y-type ions than EThcD and more c- and z●-type ions than standard AI-ETD, indicating that it is also activating some degree of ETnoD products that AI-ETD is not fully mitigating. Standard AI-ETD produces more c- and z●-type fragments than EThcD, but similar numbers of y-type fragments and fewer b-type ions.

These results show promise for both AI-ETD and AI-ETD+ in standard proteomic experiments, but they also highlight the potential AI-ETD+ has to be a new hybrid fragmentation method for challenging applications like de novo sequencing where extensive ion series and multiple fragmentation methods are highly valuable, if not necessary (Savitski et al., J. Proteome Res., 2005, 4: 2348-2354; Kim et al., Mol. Cell. Proteomics, 2010, 9: 2840-2852; and Guthals et al., J. Proteome Res., 2013, 12: 2846-2857).

### CONCLUSION

Enabling photo-dissociation capabilities on the newest generation of Orbitrap hybrid instruments offers many new exciting possibilities. The above example shows how a quadrupole-Orbitrap-linear ion trap Tribrid MS system (Orbitrap Fusion Lumos) can be easily outfitted with a continuous wave CO₂ infrared laser by affixing the laser head and optics directly to the instrument chassis for a robust implementation. Moreover, the first AI-ETD analyses was performed on the Lumos platform, demonstrating that concurrent IR photo-activation during ETD substantially improves peptide fragmentation and nearly doubles identifications in LC-MS/MS of complex peptide mixtures. With the other ETD-specific improvements already afforded by the Lumos system, this work represents the state-of-the-art ETD technology for proteomic experiments. Also introduced is a new hybrid fragmentation technique called AI-ETD+ that combines AI-ETD and IRMPD fragmentation to generate extensive c/z●- and b/y-type fragment ion series in a single MS/MS spectrum without adding any time to the scan acquisition. AI-ETD+ represents the optimal supplemental fragmentation method for ETD analyses over any other approach and enables 100% sequence coverage for the large majority of identifications it generates. This work enables new directions in research for de novo peptide sequencing, PTM characterization, intact protein analysis, as well as phosphoproteomics (Riley et al., Anal. Chem, 2017, 89: 6367-6376).

### Example 2—Phosphoproteomics with Activated Ion Electron Transfer Dissociation

The ability to localize phosphosites to specific amino acid residues is crucial to translating phosphoproteomic data into biological meaningful contexts. The following example presents the performance of AI-ETD for identifying and localizing sites of phosphorylation in both phosphopeptides and intact phosphoproteins. Using 90-minute analyses, it was demonstrated that AI-ETD can identify 24,503 localized phosphopeptide spectral matches enriched from mouse brain lysates, which more than triples identifications from standard ETD experiments and outperforms ETcaD and EThcD as well. AI-ETD achieves these gains through improved quality of fragmentation and MS/MS success rates for all precursor charge states, especially for doubly protonated species.

The degree to which phosphate neutral loss occurs from phosphopeptide product ions due to the infrared photo-activation of AI-ETD was also evaluated. Modifying phosphoRS (a phosphosite localization algorithm) to include phosphate neutral losses can significantly improve localization in AI-ETD spectra. Finally, the utility of AI-ETD was demonstrated in localizing phosphosites in α-casein, a ˜23.5 kilodalton phosphoprotein that showed eight of nine known phosphorylation sites occupied upon intact mass analysis. AI-ETD provided the greatest sequence coverage for all five charge states investigated and was the only fragmentation method to localize all eight phosphosites for each precursor. Overall, this work highlights the analytical value AI-ETD can bring to both bottom-up and top-down phosphoproteomics.

Protein phosphorylation is a dynamic post-translational modification (PTM) that is involved in a diverse array of biological regulation (Johnson et al., Biochem. Soc. Trans., 2009, 37: 627-641; and Thorner et al., Cold Spring Harb. Perspect. Biol., 2014, 6: a022913). Modern phosphoproteomic technology relies on mass spectrometry (MS) to characterize the diverse roles of phosphorylation in a wide array of biological systems (Solari et al., Mol. Biosyst., 2015, 11: 1487-1493; Engholm-Keller et al., Proteomics, 2013, 13: 910-931; Olsen et al., Mol. Cell. Proteomics, 2013, 12: 3444-3452; and Doll et al., ACS Chem. Biol., 2015, 10: 63-71). A principal advantage of MS-based phosphoproteomics is the ability to localize phosphorylation events with single residue resolution and to do so in a high-throughput and unbiased manner (Riley et al., Anal. Chem., 2016, 88: 74-94; Macek et al., Annu. Rev. Pharmacol. Toxicol., 2009, 49: 199-221; and von Stechow et al., Expert Rev. Proteomics, 2015, 12: 469-487). To achieve this goal, peptides and proteins that contain phosphoryl group(s) must undergo extensive backbone fragmentation while retaining the modification to reveal both sequence and phosphosite information. Canonical slow-heating methods like collisionally activated dissociation (CAD) have traditionally struggled with phosphorylated sequences, mainly because the phosphate loss is a low-energy dissociation pathway (Boersema et al., J. Mass Spectrom., 2009, 44: 861-878; Brodbelt et al., Anal. Chem., 2016, 88: 30-51; and Schroeder et al., Anal. Chem., 2004, 76: 3590-3598). Higher-energy collisional activation (HCD) has somewhat addressed this challenge (Nagaraj et al., J. Proteome Res., 2010, 9: 6786-6794), but alternative fragmentation methods, including electron transfer dissociation (ETD) and several photo-dissociation approaches, have also proven valuable for phosphoproteomic experiments (Chi et al., Proc. Natl. Acad. Sci. U.S.A 2007, 104, 2193-2198; Molina et al., Proc. Natl. Acad. Sci., 2007, 104: 2199-2204; Smith et al., J. Am. Soc. Mass Spectrom., 2010, 21: 2031-2040; Shaffer et al., Int. J. Mass Spectrom., 2015, 390:71-80; Lemoine et al., Rapid Commun. Mass Spectrom., 2006, 20: 507-511; Park et al., Rapid Commun. Mass Spectrom., 2009, 23: 3609-3620; Madsen et al., Chemistry, 2012, 18: 5374-5383; Kim et al., J. Am. Soc. Mass Spectrom., 2009, 20: 2334-2341; Fort et al., Anal. Chem., 2016, 88: 2303-2310; Robinson et al., J. Proteome Res., 2016, 15: 2739-2748; and Mayfield et al., ACS Chem. Biol., 2016, 12:153-162).

ETD has become especially ubiquitous as an alternative dissociation method, largely because it cleaves N—C_{α} bond along the peptide backbone to form c- and z●-type ions while preserving labile bonds like phosphorylation (Syka et al., Proc. Natl. Acad. Sci. U.S.A., 2004, 101: 9528-9533; Coon et al., Anal Chem, 2009, 81: 3208-3215; Kim et al., Proteomics, 2012, 12: 530-542; Zhurov et al., Chem. Soc. Rev., 2013, 42: 5014-5030; and Sarbu et al., Amino Acids, 2014, 46: 1625-1634). ETD is well-suited for fragmenting highly charged precursors, but fragmentation efficiency suffers for precursor ions with low charge density (Good et al., Mol. Cell. Proteomics, 2007, 6: 1942-1951), which can be problematic when requiring extensive fragmentation to confidently localize phosphosites. Poor fragmentation efficiency occurs because non-covalent intramolecular interactions are more prevalent in the more compact gas-phase structures of low charge density precursors (Laszlo et al., J. Am. Chem. Soc., 2016, 138: 9581-9588; Little et al., Anal. Chem., 1994, 66: 2809-2815; Clemmer et al., J. Am. Chem. Soc., 1995, 117: 10141-10142; and Breuker et al., J. Am. Chem. Soc., 2002, 124: 6407-6420), and these interactions hold product ions together even in the event of successful backbone cleavage, a process called non-dissociative electron transfer (ETnoD) (Pitteri et al., Anal. Chem., 2005, 77: 5662-5669; Lermyte et al., J. Am. Soc. Mass Spectrom., 2015, 26: 1068-1076; Liu et al., Int. J. Mass Spectrom. 2012, 330, 174-181; and Gunawardena et al., J. Am. Chem. Soc., 2005, 127: 12627-12639).

There are several methods to mitigate ETnoD and increase product ion yield from ETD reactions, including gentle collisional activation of ETnoD products (ETcaD) (Swaney et al., Anal Chem, 2007, 79: 477-485), beam-type collisional activation of all products of the ETD reaction (EThcD) (Frese et al., Anal. Chem., 2012, 84: 9668-9673), and infrared photo-activation concurrent with ETD reaction (activated ion-ETD, AI-ETD) (Ledvina et al., Angew Chem Int Ed Engl, 2009, 48: 8526-8528). AI-ETD is a particularly favorable approach because it significantly boosts sequence-informative fragment ion generation without increasing scan duration due to secondary activation events, and it also minimizes hydrogen rearrangements that can occur when activating the ETnoD products post-reaction (Ledvina et al., Anal. Chem., 2010, 82: 10068-10074; Ledvina et al., J. Am. Soc. Mass Spectrom., 2013, 24: 1623-1633; O'Connor et al., J. Am. Soc. Mass Spectrom., 2006, 17: 576-585; Sun et al., J. Proteome Res., 2010, 9: 6354-6367; and Xia et al., Anal. Chem., 2008, 80: 1111-1117). As discussed above, implementing AI-ETD on a quadrupole-Orbitrap-linear ion trap (q-OT-QLT, Orbitrap Fusion Lumos) significantly improves peptide identifications and quality of fragmentation in ETD experiments (Riley et al., J. Anal. Chem., 2017, 89: 6358-6366). With other ETD-specific improvements also incorporated on the Lumos platform, including a front-end ETD reagent source (Earley et al., Anal. Chem., 2013, 85: 8385-8390), high capacity ETD for improved product ion signal-to-noise (S/N) (Riley et al., J. Am. Soc. Mass Spectrom., 2016, 27: 520-531), and calibrated ETD reaction times for optimal data acquisition in shotgun proteomic analyses (Rose et al., J. Am. Soc. Mass Spectrom., 2015, 26: 1848-1857), this system represents the state-of-the art for ETD technology.

This example demonstrates how AI-ETD on the Lumos system can improve both shotgun analyses of phosphopeptides and top-down fragmentation of intact phosphoproteins. Both approaches are valuable in modern phosphoproteomics; shotgun phosphoproteomics enables the identification and quantification of thousands to tens of thousands of phosphosites in just hours of analysis time (Huttlin et al., Cell, 2010, 143: 1174-1189; Lundby et al., Nat. Commun., 2012, 3: 876; Humphrey et al., Cell Metab., 2013, 17: 1009-1020; Zhou et al., J. Proteome Res., 2013, 12: 260-271; Mertins et al., Nat. Methods, 2013, 10; 634-637; Monetti et al., Nat. Methods, 2011, 8: 655-658; Rigbolt et al., Sci. Signal., 2011, 4: rs3; Sharma et al., Cell Rep., 2014, 8: 1583-1594; Kelstrup et al., J. Proteome Res., 2014, 13: 6187-6195; Batth et al., Mol. Cell. Proteomics, 2014, 13: 2426-2434; de Graaf et al., Mol. Cell. Proteomics, 2014, 13: 2426-2434; Bian et al., J. Proteomics, 2014, 96: 253-262; Ruprecht et al., Mol. Cell. Proteomics, 2015, 14: 205-215; Giansanti et al., Cell Rep., 2015, 11: 1834-1843; Humphrey et al., Nat. Biotechnol., 2015, 33: 990-995; and Marx et al., Nat. Biotechnol., 2013, 31: 557-564). Top-down phosphoproteomics allows comprehensive proteoform characterization to localize phosphosites on a protein and show which sites are co-modified in a given proteoform (Garcia et al., J. Am. Soc. Mass Spectrom. 2010, 21: 193-202; Smith et al., Nat. Methods 2013, 10: 186-187; Sze et al., Proc. Natl. Acad. Sci. U.S.A., 2002, 99: 1774-1779; Ge et al., Proc. Natl. Acad. Sci., 2009, 106: 12658-12663; Zhang et al., J. Proteome Res., 2011, 10: 4054-4065; Zabrouskov et al., Mol. Cell. Proteomics, 2008, 7: 1838-1849; Gregorich et al., J. Proteome Res., 2016, 15: 2706-2716; Brunner et al., Anal. Chem., 2015, 87: 4152-4158; Toby et al., Annu. Rev. Anal. Chem. 2016, 9, 499-519; Good et al., J Am Soc Mass Spectrom, 2009, 20: 1435-1440; and Good et al., Proteomics, 2010, 10: 164-167). AI-ETD can triple the number of localized phosphopeptides sequenced with ETD in shotgun phosphoproteomic analyses and that it also outmatches other supplemental activation techniques, i.e., ETcaD and EThcD. Furthermore, application of AI-ETD to intact phosphoproteins was observed to be superior to other fragmentation methods for localizing multiple sites of phosphorylation on α-casein (˜23.5 kilodaltons). In all, this study presents the significant potential AI-ETD has for advancing phosphoproteomics from both the shotgun and top-down perspectives.

Materials and Methods

Sample Preparation.

Mouse brain lysates were prepared as previously described (Riley et al., J. Anal. Chem., 2017, 89: 6358-6366). The same mouse brain lysate was used for phosphopeptide enrichments as described herein, making comparisons between the two possible, as discussed in the herein. Briefly, 4 mg mouse brain was lysed and digested overnight with trypsin (Promega, Madison, Wis.). Following peptide desalting via solid phase extraction, phosphopeptides were enriched using MagResyn Ti-IMAC Ti⁴⁺-functionalized magnetic microspheres (ReSyn Biosciences, Edenvale, South Africa). Buffer A was 80% ACN with 6% trifluoroacetic acid (TFA), Buffer B was 80% ACN with 0.5 M glycolic acid, and Buffer C was 50% ACN with 1% ammonium hydroxide. 200 μL of beads were washed three times with 1 mL Buffer A. Desalted peptides were resuspended in 1 mL Buffer A, combined with the washed magnetic beads, and shaken for 20 minutes at room temperature. The beads were then washed three times with 1 mL Buffer A, once with 1 mL 100% ACN, once with 1 mL Buffer B, and once more with 1 mL Buffer B. Phosphopeptides were eluted with 300 μL Buffer C and this process was repeated for a total of two elution washes. Phosphopeptides were then dried, desalted, and resuspended in 30 μL 0.2% formic acid (FA) prior to LC-MS/MS analyses.

Shotgun LC-MS/MS.

Liquid chromatography conditions and modification to the MS system to enable AI-ETD are similar to those previously described (Riley et al., J. Anal. Chem., 2017, 89: 6358-6366). One microliter resuspended phosphopeptides was injected onto the column and gradient elution was performed at 325 nL/min, which increased from 0 to 6% B over 6 min, followed by an increase to 55% at 73 min, a ramp to 100% B at 74 min, and a wash at 100% B for the 6 min. The column was then re-equilibrated at 0% B for 10 min, for a total analysis of 90 minutes. Eluting peptides were ionized using a nanoelectrospray source held at +2 kV with respect to ground and the inlet capillary temperature was held at 275° C. Survey scans of peptide precursors were collected from 300-1350 Th with an AGC target of 5,000,000, a maximum injection time of 50 ms, and a resolution of 60,000 at 200 m/z. Monoisotopic precursor selection was enable for peptide isotopic distributions, precursors of z=2-6 were selected for data-dependent MS/MS scans for 2 seconds of cycle time, and dynamic exclusion was set to 10 seconds with a ±10 ppm window set around the precursor.

Calibrated charge dependent ETD parameters were enabled to determine ETD reagent ion AGC and ETD reaction times, and all MS/MS were mass analyzed in the Orbitrap with a resolution of 15,000 K at 200 m/z. The MS/MS AGC target value was set to 100,000 with a maximum injection time of 100 ms, and precursors were isolated with a 1.5 Th window using the quadrupole. Normalized collision energies (nce) of 35, 25, and 30 were set for ETcaD, EThcD, and HCD experiments, respectively, and AI-ETD laser powers were either 12 Watt (W) or 15 W, as indicated. For AI-ETD+ analyses, AI-ETD was performed using 15 W output from the laser head, and product ions were transferred from the high pressure cell to the low pressure cell for 2 ms of IRMPD activation at 9 W before being shuttled back to the high pressure cell to be subsequently injected to the Orbitrap for mass analysis.

Tandem mass spectra were searched using Proteome Discoverer 1.4 software. Raw files were uploaded and the spectrum selector was used to select MS/MS spectra with precursor minimum and maximum set to 350 and 10,000 Da, respectively, and a peak filter set to a minimum signal-to-noise (S/N) threshold of 1.5. A non-fragment filter was applied, removing precursor peaks within a ±1 Da window, charge reduced precursors with a ±0.5 Da window, and neutral losses from the charge reduced precursor with a ±0.5 Da window and a maximum neutral loss of 60 Da (Good et al., J Am Soc Mass Spectrom, 2009, 20: 1435-1440; and Good et al., Proteomics, 2010, 10: 164-167). The SEQUEST HT node was used to search spectra using a UniProt mouse (mus musculus) database (canonical and isoforms) with precursor mass tolerance of 50 ppm and a fragment mass tolerance of 0.2 Da (Eng et al., J. Am. Soc. Mass Spectrom., 1994, 5: 976-989). Fragment ion types searched were b-, y-, c-, and z-type for all but ETD where b-type were not included and HCD experiments where only b- and y-type fragments were used, and tryptic specificity was indicated. Carbamidomethylation of cysteine was a set as a fixed modification, and oxidation of methionine and phosphorylation of serine, threonine, and tyrosine were set as variable modifications with a max of 4 equal modifications per peptide. The Percolator node was used to filter results to a 1% false discovery rate (Kall et al., Nat. Methods, 2007, 4: 923-925; and Spivak et al., J. Proteome Res., 2009, 8: 3737-3745). phosphoRS version 3.1 was used to localize phosphosites with a 0.05 Da fragment mass tolerance (Taus et al., J. Proteome Res. 2011, 10, 5354-5362), only phosphosites with localization probabilities of 75% and higher were considered as localized and used for further anlaysis, and the phosphoRS algorithm was modified to include phosphate neutral losses in ETD spectra as discussed in the text. Three technical replicate analyses were batched together for each method.

Top-Down Analysis.

A-casein was purchased as a mass spectrometry grade standard from Protea Biosciences (Morgantown, W. Va.) and was resuspended at 10 pmol/μL in 50% ACN/49.8% H₂O, and 0.2% FA. The protein solution was infused via syringe pump into the mass spectrometer at 5 μL/min using a 500 μL syringe and precursors were ionized with electrospray ionization (ESI) at 4.5 kV with respect to ground. Intact protein mode was enabled to reduced nitrogen pressure in the ion-routing multipole to 3 mTorr, and full MS spectra were collected in the Orbitrap at a resolution of 240,000 K at 200 m/z with an AGC target value of 1,000,000. MS/MS scans were performed in the Orbitrap at a resolution of 240,000 at 200 m/z with an AGC target value of 800,000.

Precursors were isolated with the mass selecting quadrupole using an isolation width of 5 m/z, and 200 transients were averaged. HCD collision energies ranged from 15-20 nce. An AGC target of 300,000 charges was used for fluoranthene reagent anions (m/z 202, isolated by the mass selecting quadrupole) for ETD, EThcD, and AI-ETD experiments. ETD reaction times ranged from 12-30 ms depending on precursor charge state, EThcD collision energies were either 12 or 15 nce, and AI-ETD laser powers were either 18 or 21 W. Multiple nce values and laser powers were tested for EThcD and AI-ETD, respectively, to determine optimal performance (data not shown). MS/MS spectra were deconvoluted with XTRACT (Thermo Fisher Scientific) using default parameters and a S/N threshold of three. ProSight Lite was used to generate matched fragments using a 10 ppm tolerance (Fellers et al., Proteomics, 2014, 15:1235-1238). All ETD, EThcD, and AI-ETD spectra were matched with c-, z-, b-, and y-type ions while HCD spectra were matched with b- and y-type fragments. Phosphoserines modified in α-casein were identified using known sites at the UniProt resource (accession P02663).

Results and Discussion

AI-ETD for Bottom-Up Phosphoproteomics.

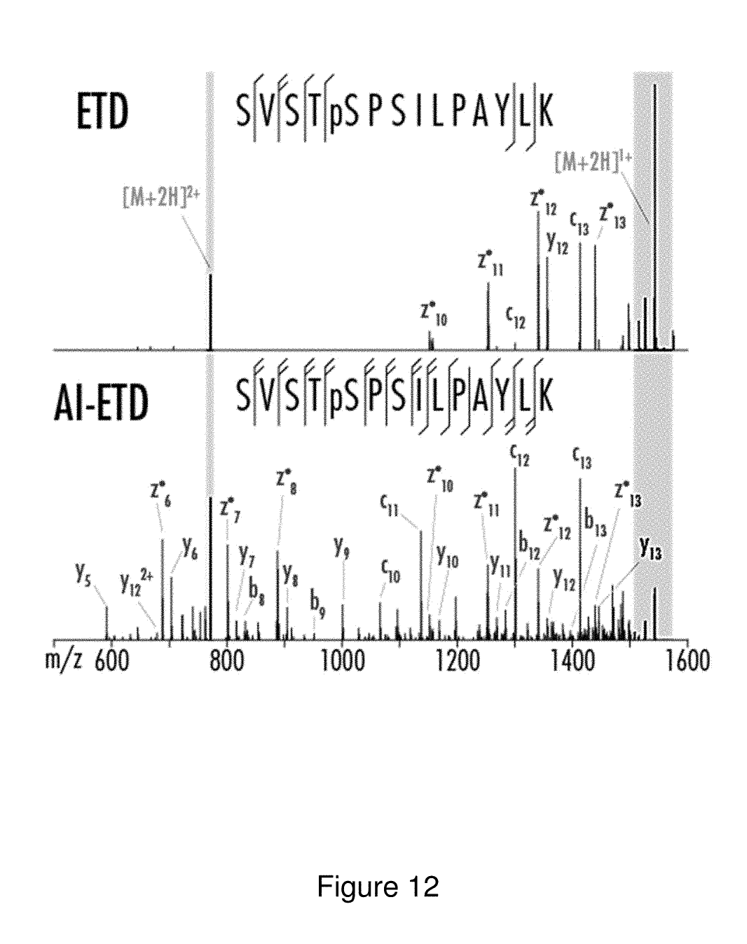

AI-ETD improves ETD dissociation efficiency by using IR photo-activation concurrent with the ion-ion reaction to disrupt non-covalent interactions and drive the formation of sequence-informative product ions. To evaluate AI-ETD for shotgun phosphoproteomics, 90-minute LC-MS/MS analyses were conducted on complex mixtures of tryptic phosphopeptides enriched from mouse brain lysate. FIG. 12 shows an example comparison of ETD and AI-ETD spectra for the doubly protonated precursor of a phosphopeptide having the sequence SVSTpSPSILPAYLK, which was identified by both fragmentation methods in the LC-MS/MS analyses. This 14 residue phosphopeptide has one phosphorylated residue but six potential sites of modification (S/T/Y). ETD generated seven total sequence-informative fragment ions that correspond to cleavage of six of the 13 total inter-residue positions (˜46% sequence coverage), eliminating three potential phosphosites but ultimately failing to localize the correct site of modification. AI-ETD, on the other hand, provided comprehensive fragmentation, producing 25 b-, y-, c- and z●-type product ions. AI-ETD is known to produce c, z●-, and y-type fragments (also seen in standard ETD) with minimal addition of b-type products for unmodified peptides, but b- and y-type ions are more prevalent in AI-ETD spectra of phosphopeptides, as seen here. Below we discuss this phenomenon and how to make use of it in data analysis phosphopeptide AI-ETD spectra. Overall, AI-ETD generates product ions for every inter-residue position for 100% sequence coverage and confident localization of the phosphosite to the serine at position five in the peptide sequence.

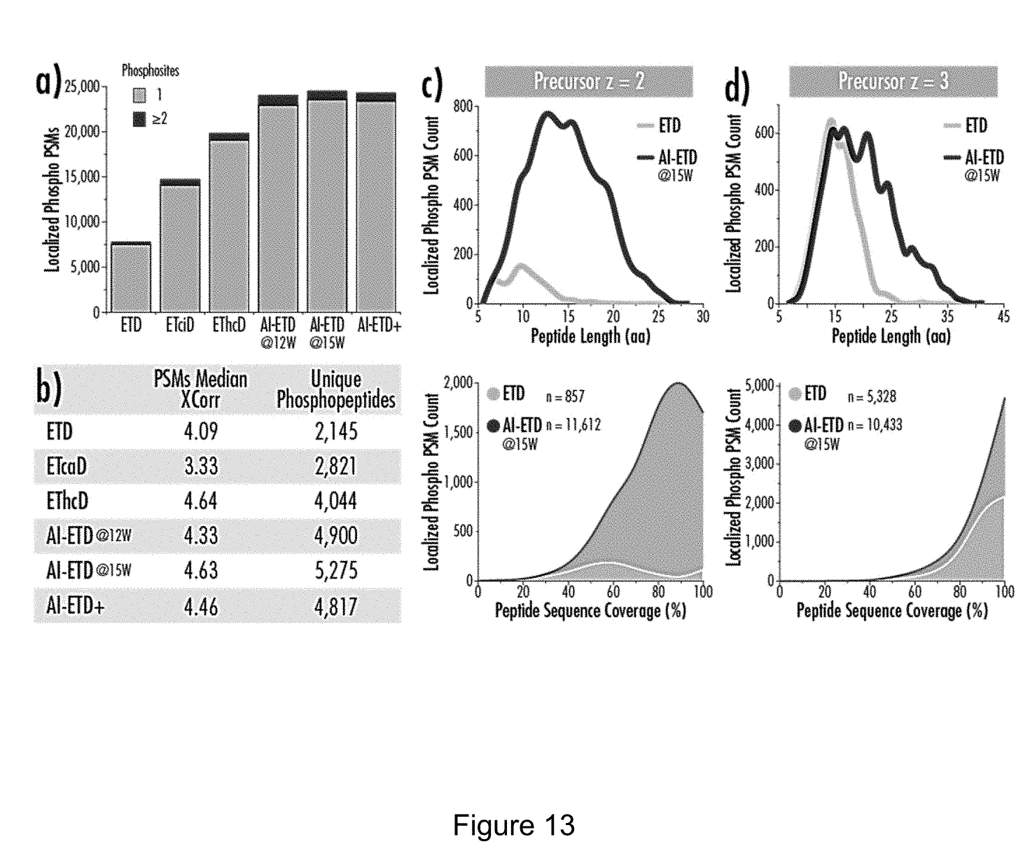

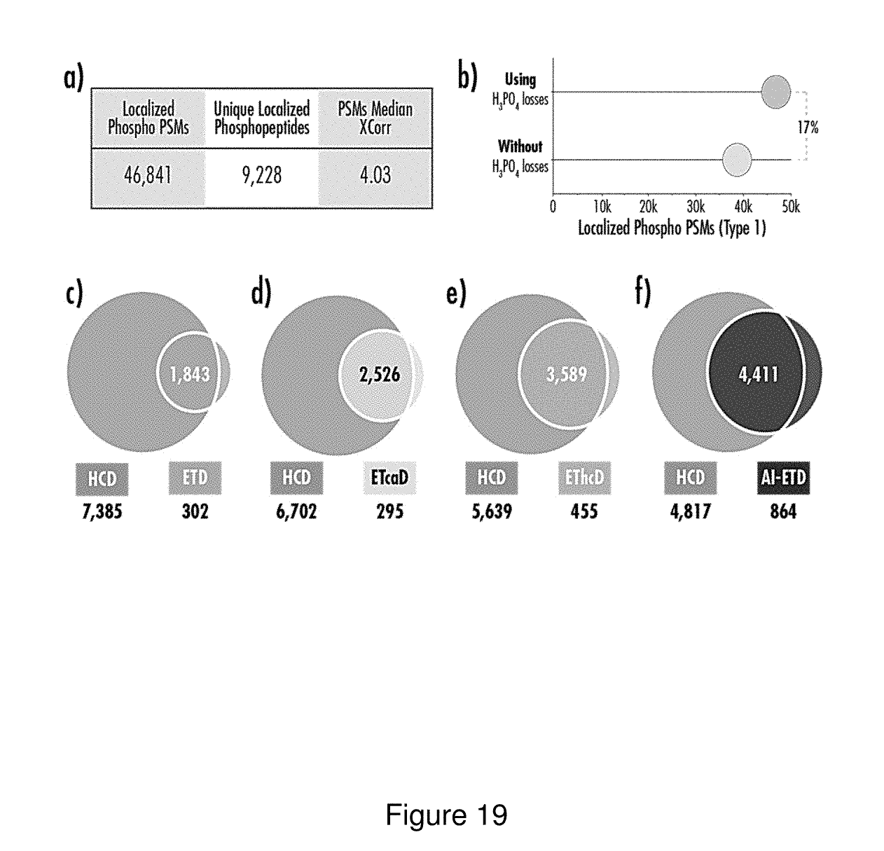

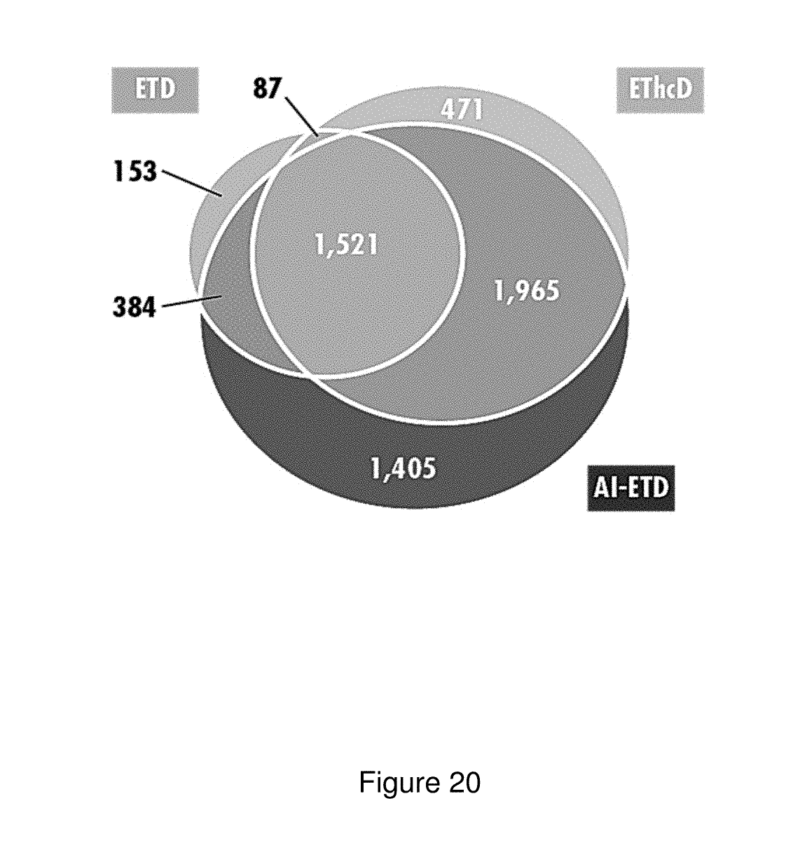

FIG. 13 summarizes the data from the shotgun analyses for ETD, ETcaD, EThcD, AI-ETD at two different laser powers, and with AI-ETD+. As described above, AI-ETD+ is a novel hybrid fragmentation approach that combines AI-ETD with a second short (several ms) photo-activation in the low pressure cell of the QLT. This secondary activation can induce infrared multi photon dissociation (IRMPD) to produce b- and y-type fragments in addition to the c, z●-, and y-type fragments generated by AI-ETD. Overall AI-ETD (15 W) provides the greatest number of localized PSMs and unique phosphopeptides (FIG. 13, panels a and b), more than tripling the number of phospho peptide spectral matches (phospho PSMs) generated by ETD. EThcD, known to perform well for phosphopeptide fragmentation and phosphosite localization (Frese et al., J. Proteome Res. 2013, 12, 1520-1525; and Mommen et al., Proc. Natl. Acad. Sci. U.S.A., 2014, 111: 4507-4512), and ETcaD, also both significantly increase phospho PSM identifications over ETD with increases of 2.6-fold and 1.9-fold, respectively. EThcD and AI-ETD generate approximately equivalent median SEQUEST Xcorr values, a measure of spectral match quality. Both improve upon the median score seen with ETD even with the large increase in number of phospho PSMs, illustrating that the phospho PSMs gained are high quality spectra. Surprisingly, AI-ETD+, which proved to be the optimal supplemental activation approach for non-modified peptides, underperformed compared to AI-ETD, indicating that the additional IRMPD activation may have contributed to over-fragmentation. It is noted that HCD outperformed all ETD-based analyses, but AI-ETD provided the greatest number of unique phosphopeptides when comparing the overlap of HCD and ETD-based methods (FIG. 19). AI-ETD also provided 1,405 unique phosphopeptide identifications when evaluating the overlap in identifications between ETD, EThcD, and AI-ETD, compared to 153 and 471 unique phosphopeptides with ETD and EThcD, respectively (FIG. 20).

To further evaluate the improvements of AI-ETD over ETD, the length of phosphopeptides sequenced by both methods were compared and the sequence coverage achieved for both doubly and triply protonated precursors (FIG. 13, panels c and d, respectively). Sequence coverage is defined herein as the ratio of [inter-residue positions that can be explained by observed product ions (b-, y-, c- or z●-type)] to [the total number of inter-residue positions (i.e., the number of residues minus 1)]. This number is reported as a percentage and the maximum sequence coverage attainable is by default 100%. AI-ETD generated ˜10,000 more phospho PSMs for doubly protonated precursors than ETD and significantly increased the length of doubly charge phosphopeptides that were successfully sequenced. The majority of z=+2 phosphopeptides identified with AI-ETD also had sequence coverages of 80-100%. ETD performed more favorably for z=+3 precursors, but it still produced less than half of the identifications of AI-ETD, which successfully sequenced longer peptides than ETD while maintaining >80% sequence coverage for the majority of precursors.

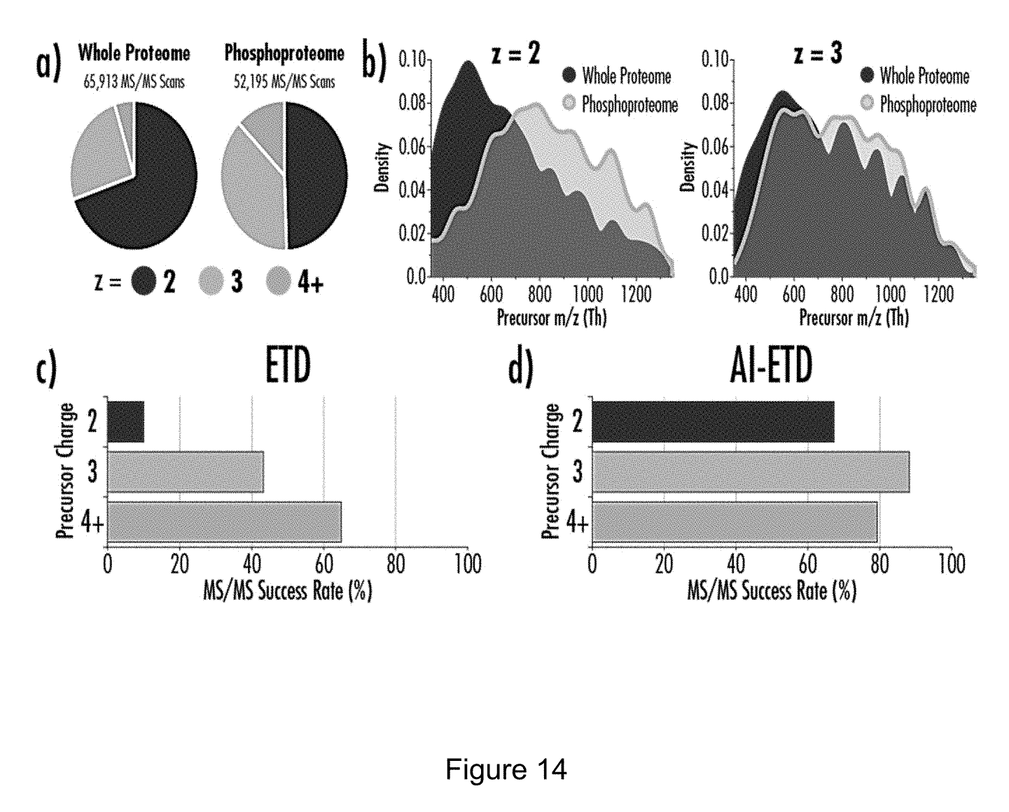

To understand why AI-ETD soundly outperformed ETD, LC-MS/MS data was compared from non-enriched “whole” proteome samples to the phosphoproteome samples described here (FIG. 14). The proteome data comes experiments performing AI-ETD on the Lumos, and the peptides in that data set come from the same mouse brain lysate that was used for phosphopeptide enrichment for this study, making comparisons straight-forward. The charge state distribution of precursors selected for MS/MS fragmentation shifts to higher charge states in the phosphoproteomic experiments (FIG. 14, panel a), which should benefit ETD; however, precursor m/z also shifts substantially to higher m/z values for doubly protonated precursors, with a less significant but still noticeable shift to higher m/z values for z=+3 precursors (FIG. 14, panel b). Thus, even with a wider range of charge states observed, precursors are shifted to lower charge densities overall, ultimately hindering ETD efficiency. These changes in precursor populations have been seen in other studies and are likely due not only to the addition of the modification mass but also to longer peptides that occur because of phosphorylation-based hindrance of trypsin digestion (Dickhut et al., J. Proteome Res., 2014, 13: 2761-2770). Furthermore, if a phosphate group is deprotonated, the net charge of a peptide may be reduced, generating longer peptides at lower charge states (i.e., more peptides with lower charge density).

Having the majority of precursors >600 m/z is a consequential disadvantage for ETD but does not affect AI-ETD, which is reflected in the MS/MS success rates for precursors with different charge states between the two methods (FIG. 14, panels c and d). MS/MS success rate, reported as a percent, is defined as the number of PSMs successfully identified divided by the total number of MS/MS scans. AI-ETD maintains success rates above 60% for all precursor charge states, with a greater than 6-fold increase in success rate for z=+2 precursors. The clear improvement in success rates for all precursor charge states explains the more than 3-fold increase in phospho PSMs with AI-ETD.

Evaluating Phosphopeptide Fragmentation with AI-ETD.

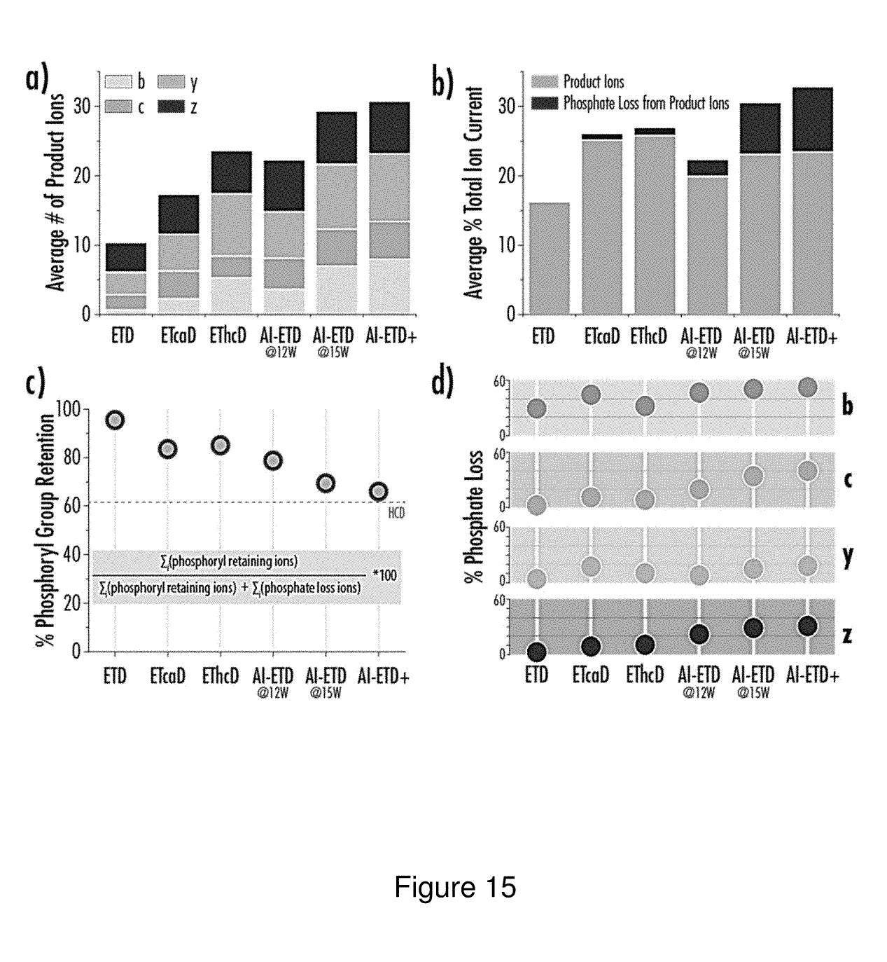

The ETD, ETcaD, EThcD, AI-ETD, and AI-ETD+ fragmentation of phosphopeptides identified from tens of thousands of MS/MS spectra from shotgun experiments were investigated, focusing on z=+2 precursors (FIG. 15). Phosphoryl groups are chromophores for the 10.6 μm IR photons used in AI-ETD, making phosphopeptide fragmentation with AI-ETD particularly interesting (Crowe et al., J. Am. Soc. Mass Spectrom., 2004, 15: 1581-1592). IRMPD studies have shown that phosphopeptides can be selectively fragmented over non-modified peptides because the phosphoryl moieties readily absorb IR photons, and the b- and y-type product ions formed by this by this fragmentation can both retain phosphoryl groups or undergo neutral loss (Crowe et al., Anal. Chem., 2005, 77: 5726-5734; Flora et al., Anal. Chem., 2001, 73: 3305-3311; Flora et al., J. Am. Chem. Soc. 2002, 124: 6546-6547; and Vasicek et al., J. Am. Soc. Mass Spectrom., 2011, 22: 1105-1108). Previous work has also shown that AI-ETD on phosphopeptides with lowered bath gas pressures can generate b- and y-type ions from IRMPD-like activation. Here it is shown that b- and y-type products from IRMPD are present in AI-ETD spectra of phosphopeptides under normal operating conditions on the Lumos system, and it is demonstrated that phosphate neutral losses caused by IRMPD can be used by a localization algorithm to improve phosphosite localization.

FIG. 15, panel a, presents the average number of product ions identified in MS/MS spectra of the different fragmentation methods, with the count being delineated by fragment ion type. EThcD significantly increases the number of b- and y-type ions generated while marginally increasing the number of c- and z●-type products, matching previous studies (Ledvina et al., J. Am. Soc. Mass Spectrom., 2013, 24: 1623-1633; and Frese et al., J. Proteome Res., 2013, 12: 1520-1525). AI-ETD (15 W) further improves upon c- and z●-type ion generation and produces even more b- and y-type fragments than EThcD. This differs from AI-ETD of non-modified peptides and is likely due to the increased internal energy of the precursors as phosphoryl groups absorb IR photons. AI-ETD+, which includes a short IRMPD activation step in the low pressure cell of the QLT, produces similar numbers of c- and z●-type ions to AI-ETD but generates the highest number of b- and y-type ions. Note, the fragment ion count includes intact b-, y-, c- and z●-type products and phosphate neutral losses from those ions. FIG. 15, panel b, shows the average percent of total signal (total ion current) accounted for in b-, y-, c- and z●-type products and indicates how much of that signal comes from product ions that show phosphate neutral loss.

Clearly AI-ETD and AI-ETD+ generate considerable phosphate neutral loss from fragment ions, but a significant population of product ions retain their phosphoryl groups as well, as is expected with ETD-like fragmentation. A percent of total phosphoryl group retention was claculated, analogous to the calculations performed for UVPD spectra of phosphopeptides (Robinson et al., J. Proteome Res., 2016, 15: 2739-2748), to understand what fraction of product ions retain their phosphate moiety under different conditions (FIG. 15, panel c). Percent phosphoryl group retention represents the ratio of product ions (i.e., b-, y-, c- and z●-type) that retain the phosphoryl group to the total number of product ions that have the phosphorylation modification based on position in the phosphopeptide sequence. Nearly all product ions retain phosphoyl groups in ETD, while ˜80% of product ions retain the modification in ETciD, EThcD, and lower laser power AI-ETD. AI-ETD (15 W), however, shows more a more significant drop in product ion phosphoryl group retention, and the phosphoryl group retention in AI-ETD+ is lowest, nearly matching the amount of loss seen in HCD spectra. FIG. 15, panel d, shows the percent phosphate loss (i.e., the complement to phosphate retention, or 100-percent phosphoryl group retention) delineated by product ion type. For all fragmentation methods, if b-type ions are present, they are more likely to show phosphate loss than any other ion. Surprisingly, c- and z●-type product ions show the most significant phosphate loss in AI-ETD and AI-ETD+, a phenomenon that has not been reported.

Although phosphate neutral loss from product ions is not ideal, it is not debilitating. HCD spectra have considerable phosphate neutral losses from b- and y-type product ions, but phosphosite localization is still very much achievable in these cases (Taus et al., J. Proteome Res., 2011, 10: 5354-5362; Savitski et al., Mol. Cell. Proteomics, 2011, 10: M110.003830; and Beausoleil et al., Nat. Biotechnol., 2006, 24: 1285-1292). In fact, many phosphosite localization algorithms use phosphate neutral losses to aid in the localization process (FIG. 19). The phosphoRS algorithm was modified for EThcD fragmentation to include phosphate neutral losses from b- and y-type ions in the localization calculation, but no algorithm to date has had reason to include phosphate losses from c- and z●-type fragments.

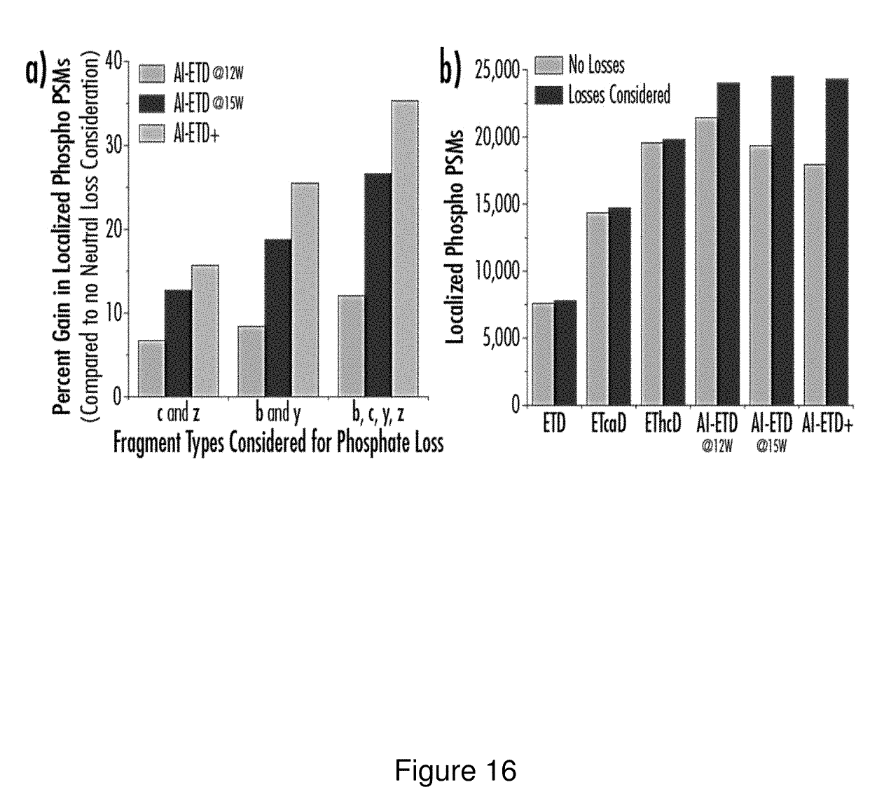

It is reasoned that localization in AI-ETD spectra could be improved by modifying the phosphoRS algorithm to utilize neutral losses from all four fragment ion types in its calculation, analogous to how it currently uses neutral losses for HCD-type spectra. FIG. 16, panel a, presents the percent gain in phospho PSMs for AI-ETD and AI-ETD+ when considering phosphate neutral loss from three different sets for product ions compared to localizing phosphosites without any consideration of phosphate neutral losses. Moderate gains were seen for AI-ETD (12 W) for each of the three fragment ion sets (c and z; b and y; and all four), but more considerable increases were achieved for AI-ETD (15 W) and AI-ETD+, matching the trends in FIG. 15. FIG. 16, panel b, shows the difference in localized phospho PSMs for ETD and the different supplemental activation methods when considering phosphate neutral losses from all four fragment ion types (dark grey) or when not considering any neutral losses (light grey). The results from considering neutral losses is presented in FIG. 13a. The gains in number of localized phospho PSMs are significant for AI-ETD and AI-ETD+, demonstrating that modifications to localization software to consider these features of AI-ETD spectra of phosphopeptides is of value.

AI-ETD for Top-Down Phosphoproteomics.

Extensive backbone fragmentation is equally, if not more, important when trying to localize phosphosites on intact proteins as the number of potential modification sites greatly increases with longer amino acid chains. AI-ETD improves fragmentation and sequence coverage for top-down proteomics (Riley et al., Anal. Chem., 2015, 87: 7109-7116; Zhao et al., Anal. Chem., 2015, 87: 5422-5429 and Bourgoin-Voillard et al., Proteomics, 2014, 14: 1174-1184), but it has yet to be applied to intact phosphoproteins. To investigate the utility of AI-ETD for top-down phosphoproteomics, five charge states of α-casein spanning the charge state envelope of the protein (z=+22, +20, +18, +16, and +14) were fragmented and performance compared to HCD, ETD, and EThcD. The most dominant precursor species contained eight sites of phosphorylation, although nine known sites of phosphorylation and 31 potential sites (S/T/Y) exist.

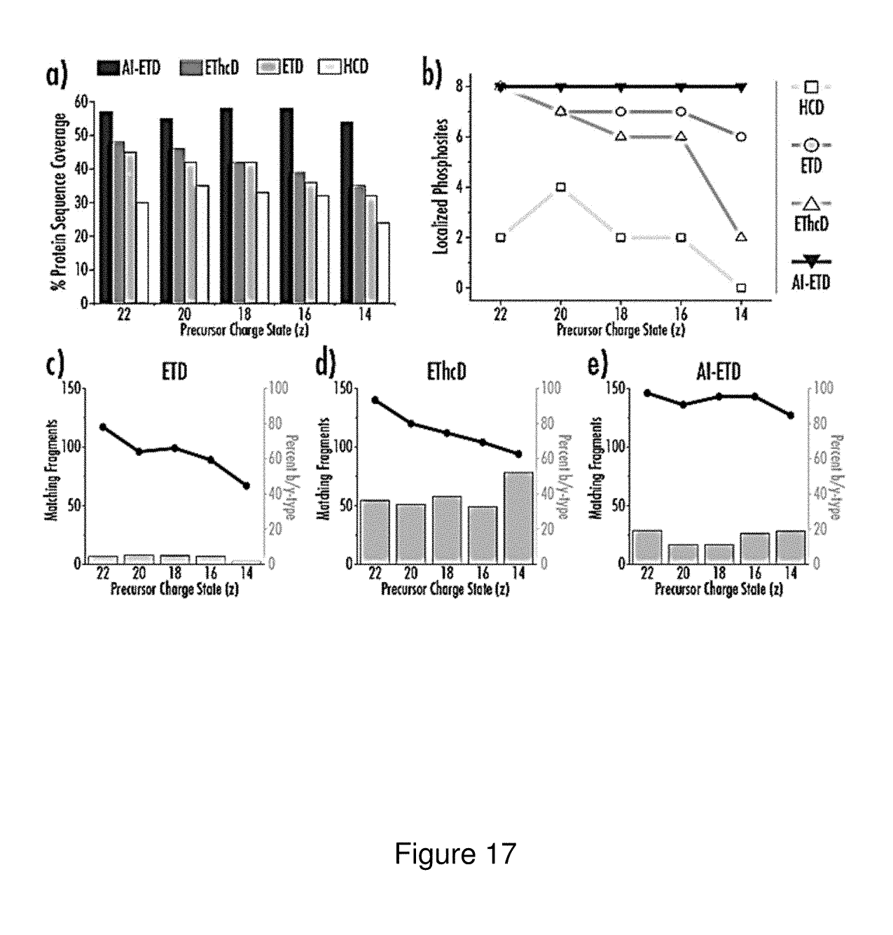

FIG. 17, panel a, reports the sequence coverage achieved with each fragmentation method for each of the five precursors selected. AI-ETD provides the highest sequence for each charge state, even outperforming EThcD, which is known to aid in sequence coverage and phosphosite localization (Brunner et al., Anal. Chem. 2015, 87: 4152-4158). AI-ETD is the only fragmentation method to provide unambiguous localization of all eight sites of phosphorylation for each of the five precursors, although ETD also performed well (FIG. 17, panel b). AI-ETD generates more fragment ions than ETD and EThcD for all precursors, too (FIG. 17, panels c-e, grey line plots, left axes). Knowing how AI-ETD affected product ion generation in phosphopeptides, the percentage of matching fragments (i.e., the number of fragments matched by ProSight Lite) that were c/z●-type versus b/y-type was investigated (FIG. 17, panels c-e, gold bars, right axes). Nearly all fragment ions formed by ETD were c/z●-type, i.e., less than ˜5% of the total sequence-informative product ions were b/y-type ions. EThcD substantially increase the ratio of b/y-type ions formed, with ˜30-40% of all matched fragments being b/y-type and 52% of fragments of being b/y-type for the z=+14 precursor, which agrees with previous investigations (Brunner et al., Anal. Chem. 2015, 87: 4152-4158). AI-ETD noticeably increases the ratio of b/y-type ions (˜10-20%) over ETD, but is only roughly half of the ratio seen with EThcD. This differs from the phosphopeptide fragmentation data above, likely because the increase in internal energy that occurs when phosphoryl moieties absorb IR photons is distributed along a much longer amino acid sequence for intact proteins, meaning the overall energy of the precursor ions is not increased enough to induce as much collisional activation into b/y-type products.

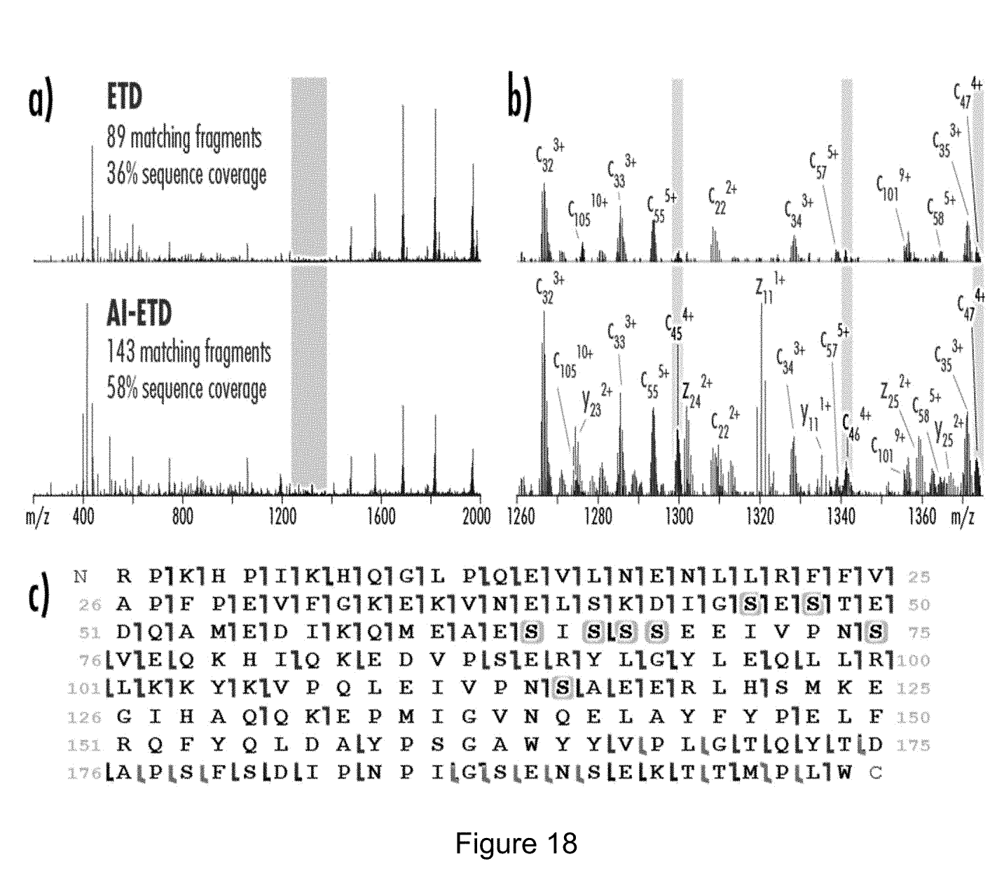

The benefits of AI-ETD are clear when comparing ETD and AI-ETD spectra (FIG. 18). AI-ETD decreases the amount of charge-reduced precursor (dominant peaks from 1400-2000 m/z) compared to ETD while boosting the number and abundance of product ions (FIG. 18, panel a). A zoom in of the spectra (FIG. 18, panel b, representative of the grey region in part a) shows that AI-ETD increases signal of product ions detected in ETD while also generating new product ions. Formation of new c-, z-, and y-type ions are easily distinguished in the AI-ETD spectrum, too. The product ions highlighted in yellow (c₄₅⁴⁺, c₄₆⁴⁺, and c₄₇⁴⁺) were critical in localizing the phosphosite on serine 46. All three c-type fragments are present in the AI-ETD spectrum, but c₄₅⁴⁺ and c₄₆⁴⁺ are either not present or have too low S/N for effective deconvolution in the ETD spectrum. ETD and AI-ETD spectra are on the same intensity scale for both parts a and b. FIG. 18, panel c, depicts a sequence coverage map for AI-ETD of z=+16 precursor of α-casein, showing the confident localization of all eight phosphosites of the modified precursor.

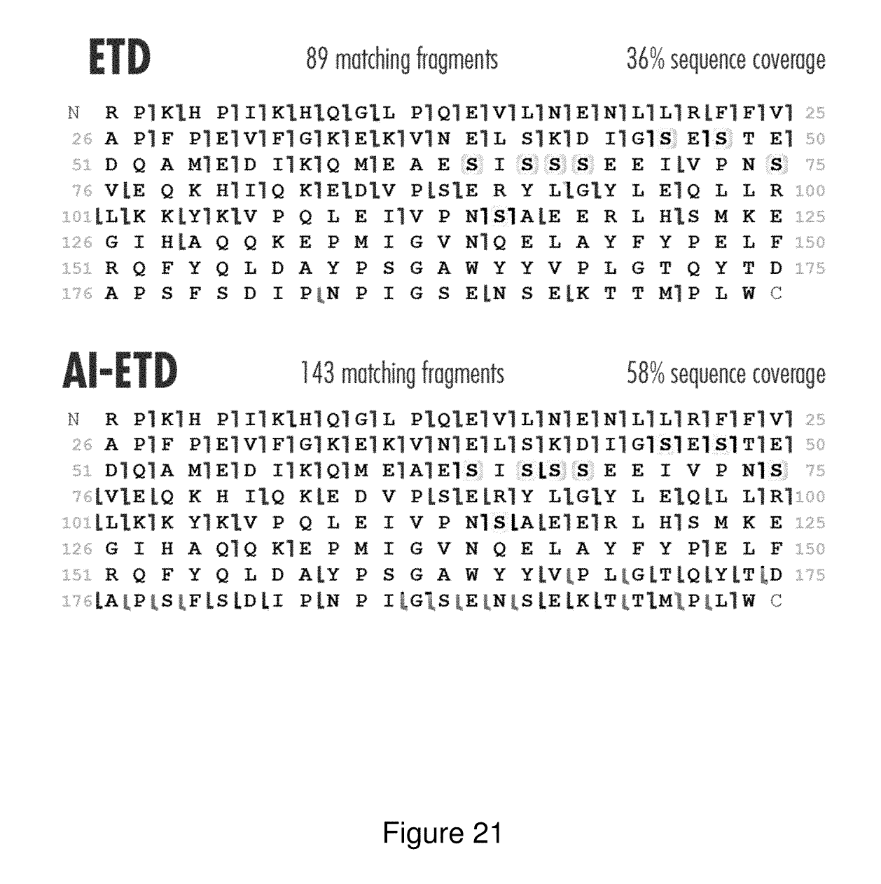

FIG. 21 compares sequence coverage maps from ETD to AI-ETD data. Interestingly, the N-terminal portion of the sequence is well characterized by both fragmentation methods, whereas AI-ETD provides more fragmentation in the middle and C-terminal regions of the protein. The phosphorylated residues are bunched in the middle of the sequence (or slightly N-terminal of middle), where ETD can still provide proficient sequence coverage (although it fails to confidently localize all phosphosites). This may indicate that the high density of negative charge at the cluster of phosphosites may contribute to a relatively unfolded secondary gas-phase structure in this region of the backbone while C-terminal stretch of the sequence remains folded due to less charge repulsion, although this would need to be followed up with subsequent studies to confirm. Note, α-casein was resuspended and electrosprayed in denaturing conditions, meaning any gas-phase structure that could be discerned may not be relevant to in-solution structure.

The ability of AI-ETD to produce high quality data across a wide range of precursor charge states and m/z is extremely beneficial because it mitigates the reliance on exclusively high charge density (i.e., highly charged, low m/z) precursors while still providing extensive sequence coverage that is a hallmark of ETD for intact proteins.

### CONCLUSION

As discussed above, AI-ETD can be implemented in a straight-forward and robust manner on a quadrupole-Orbitrap-linear ion trap hybrid MS system (Orbitrap Fusion Lumos). Her it is demonstrated that AI-ETD can significantly improve phosphoproteomic analyses for both shotgun and top-down approaches. AI-ETD more than triples the number of localized phospho PSMs sequenced by ETD, which corresponds to a nearly 2.5-fold increase in unique localized phosphopeptides identified. These gains are realized because AI-ETD improves phosphopeptide fragmentation at all precursor charge states compared to ETD, producing substantially more product ions and providing much greater peptide sequence coverage.

AI-ETD of phosphopeptides was also shown to produce more b- and y-type ions than expected, in addition to increased generation of c- and z●-type fragments. Phosphate neutral losses from all four fragment ion types were observed with AI-ETD, including losses from c- and z●-type fragments, which was surprising. These neutral loss products can be used by modified version of phosphoRS to improve phosphosite localization in AI-ETD spectra, which provides a blueprint for modifying other localization software to best utilize the rich fragmentation produced by AI-ETD. Finally, it was demonstrated that AI-ETD is the superior fragmentation method for sequencing the multiply-phosphorylated protein α-casein, where AI-ETD provided the highest sequence coverage and best ability to localize phosphosites over HCD, ETD, and EThcD. AI-ETD is poised to be a premier method for top-down phosphoproteomics.

Several studies have shown the negative mode to an interesting avenue to pursue for characterizing the phosphoproteome (Robinson et al., J. Proteome Res., 2016, 15: 2739-2748; Flora et al., Anal. Chem., 2001, 73, 3305-3311; Robinson et al., J. Am. Soc. Mass Spectrom., 2014, 25: 1461-1471; Huzarska et al., Anal. Chem., 2010, 82: 2873-2878; and Madsen et al., Proteomics, 2011, 11: 1329-1334). The present invention moves the field one step closer to implementing activated ion negative electron transfer dissociation for phosphoproteomic analyses, which would enable exciting new dimensions to phosphoproteome analysis (McAlister et al., Anal. Chem., 2012, 84: 2875-2882; Riley et al., Mol. Cell. Proteomics, 2015, 14: 2644-2660; Riley et al., J. Proteome Res., 2016, 15: 2768-2776; Shaw et al., Anal. Chem., 2013, 85: 4721-4728; and Rush et al., J Am Soc Mass Spectrom, 2017, 28: 1324-1332). In all, this work both shows that AI-ETD is becoming a more accessible tool for the proteomics community and also opens the door to new frontiers of research with this technique. It can bring tangible benefits to challenging problems in phosphoproteomics and other PTM analyses and serve as another avenue to translating MS-based phosphoproteomic analyses into valuable biological contexts.

### Example 3—Characterization of Peptides with AI-ETD

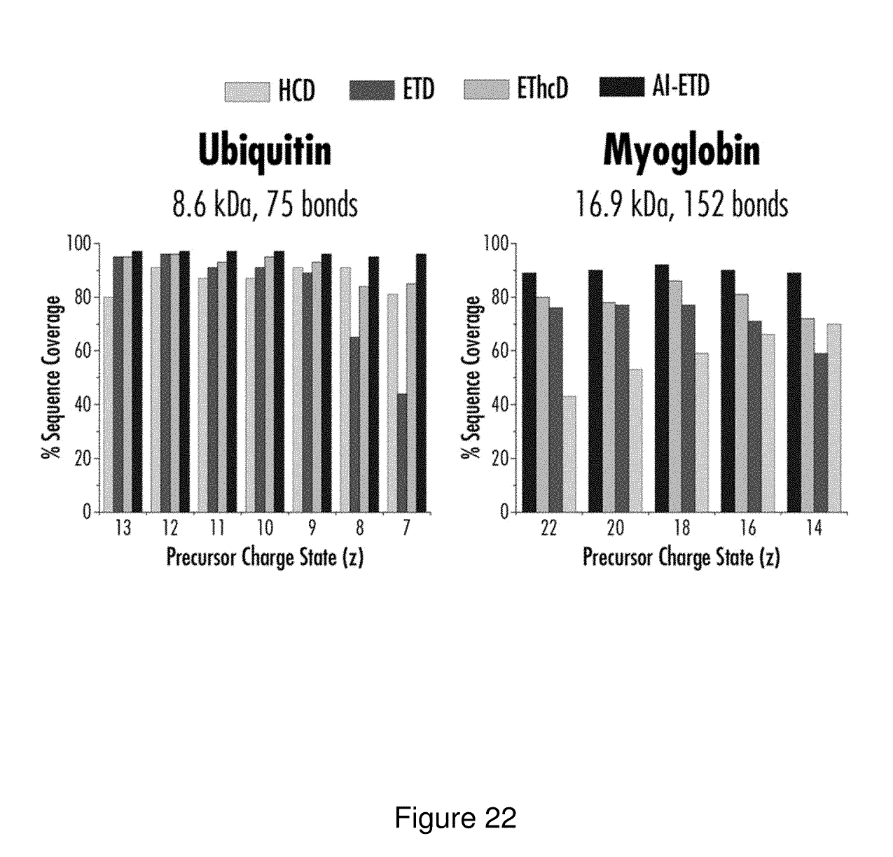

FIG. 22 shows a comparison of sequence coverage of two proteins, ubiquitin and myoglobin, using HCD, ETD, EThcD, and AI-ETD as described above. As seen in these figures, AI-ETD had the greatest percentage of sequence coverage.

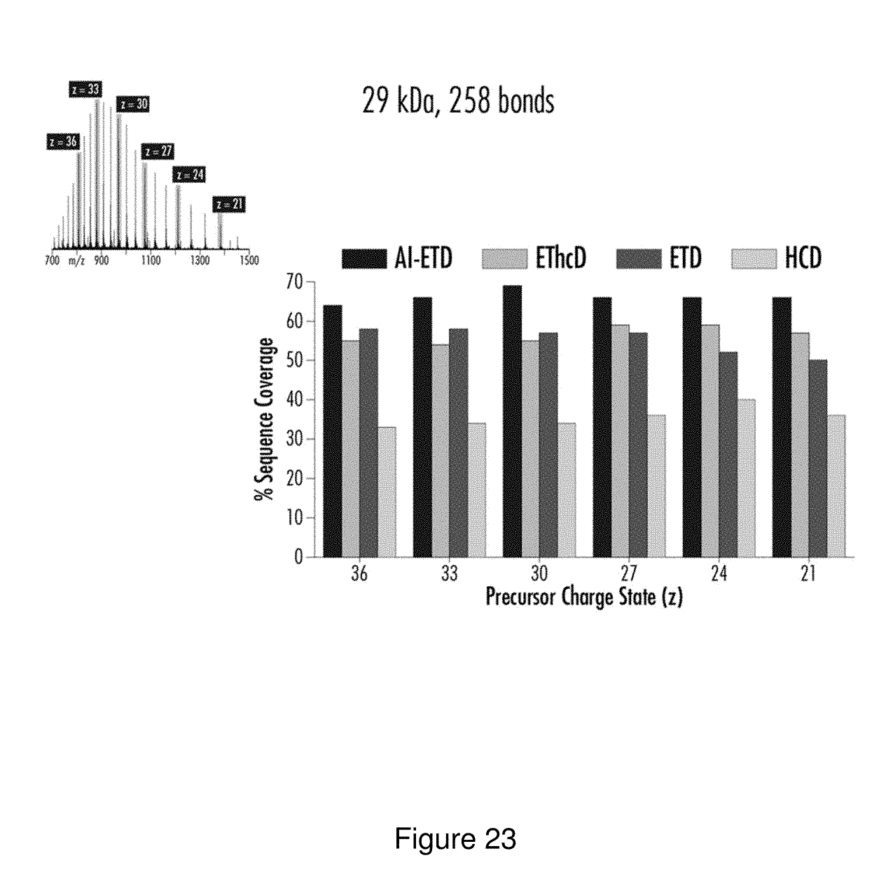

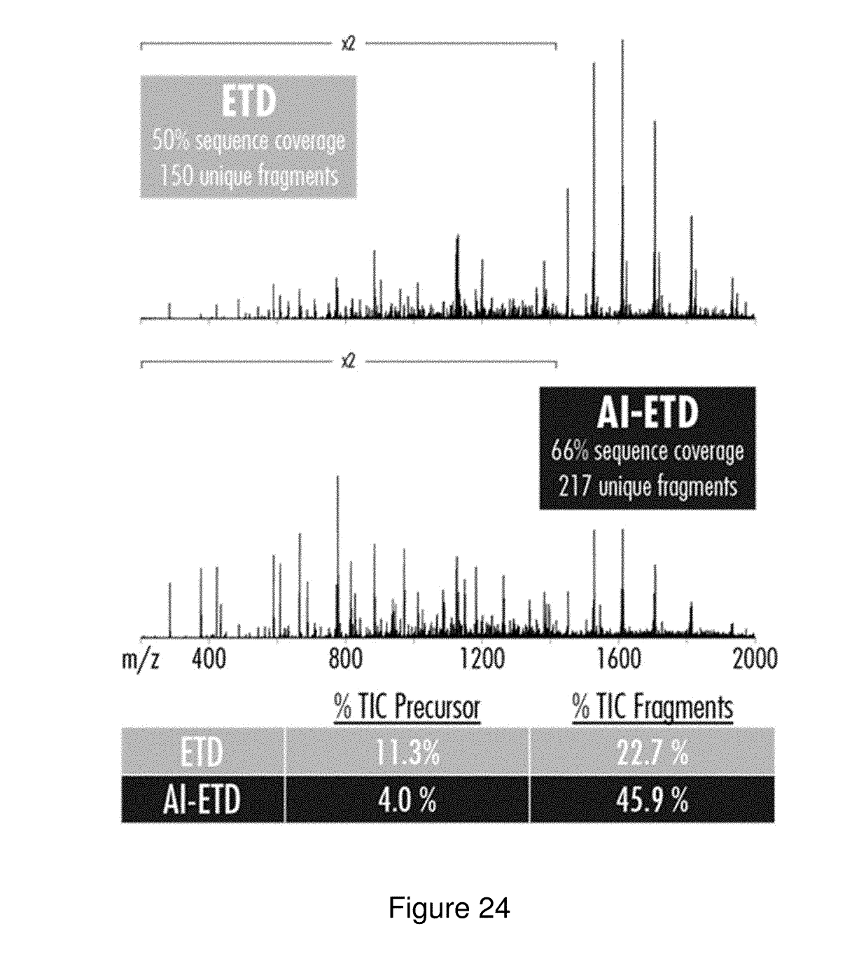

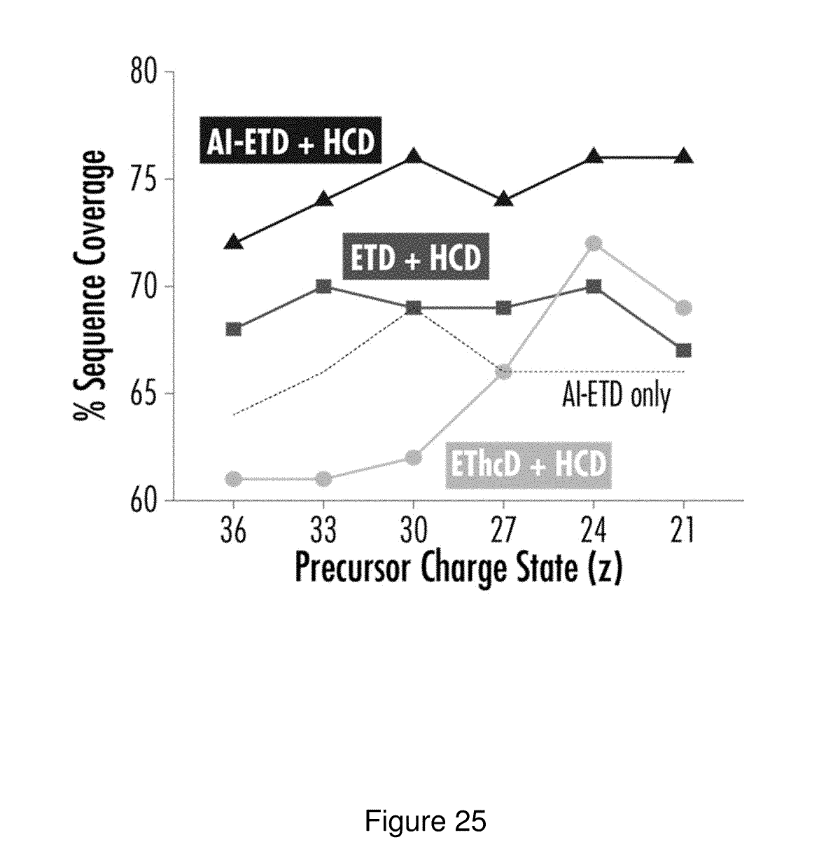

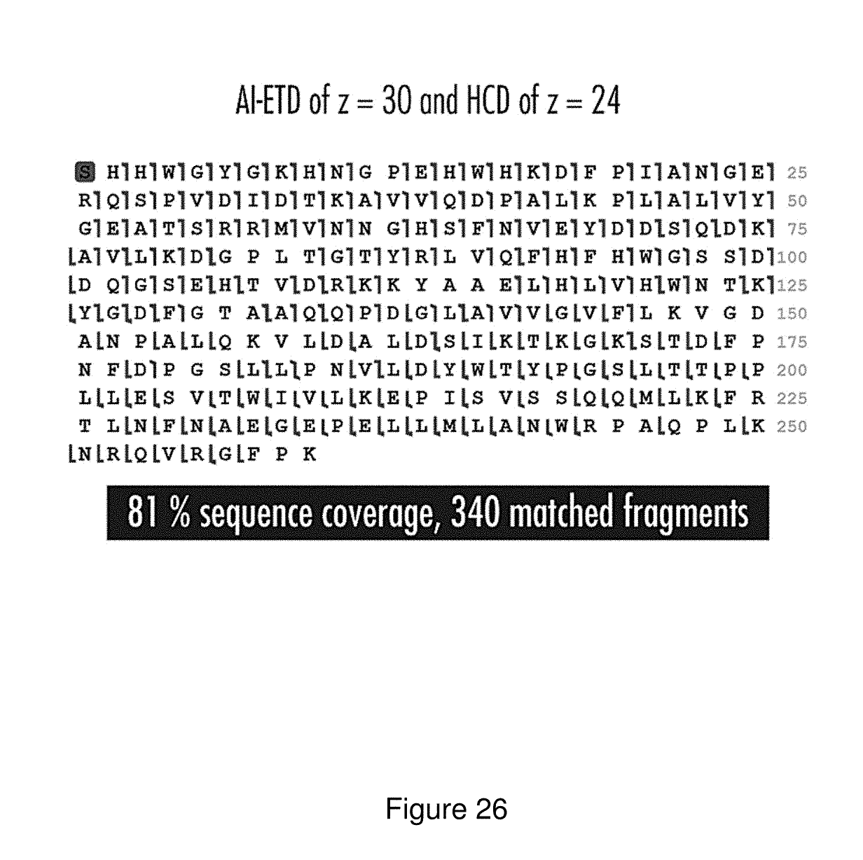

FIGS. 23-26 similarly illustrate the sequence coverage of carbonic anhydrase. As seen in FIG. 24, AI-ETD resulted in greater sequence coverage and a greater number of unique fragments. When AI-ETD, ETD, and EThcD are used in conjunction with HCD, the sequence coverage is increased with AI-ETD+ HCD providing the greatest amount of sequence coverage (FIG. 25). In an experiment where AI-ETD was performed using a charge state of 30 and HCD performed with a charge state of 24, 81% sequence coverage was obtained (FIG. 26).

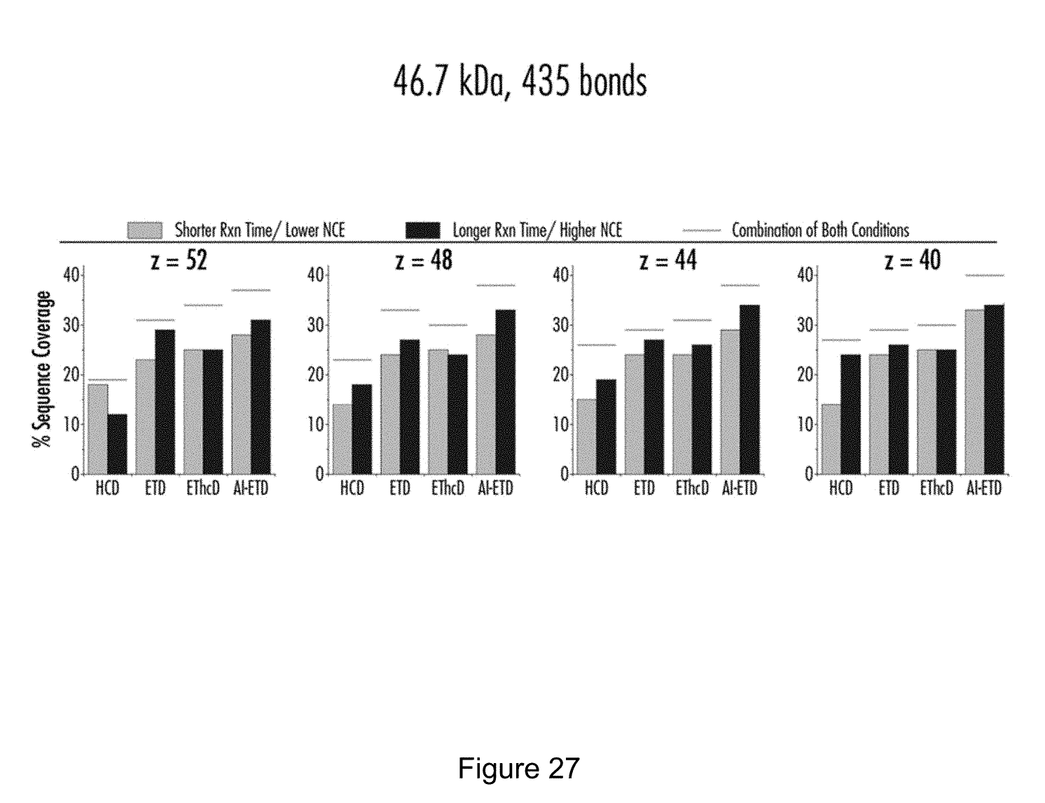

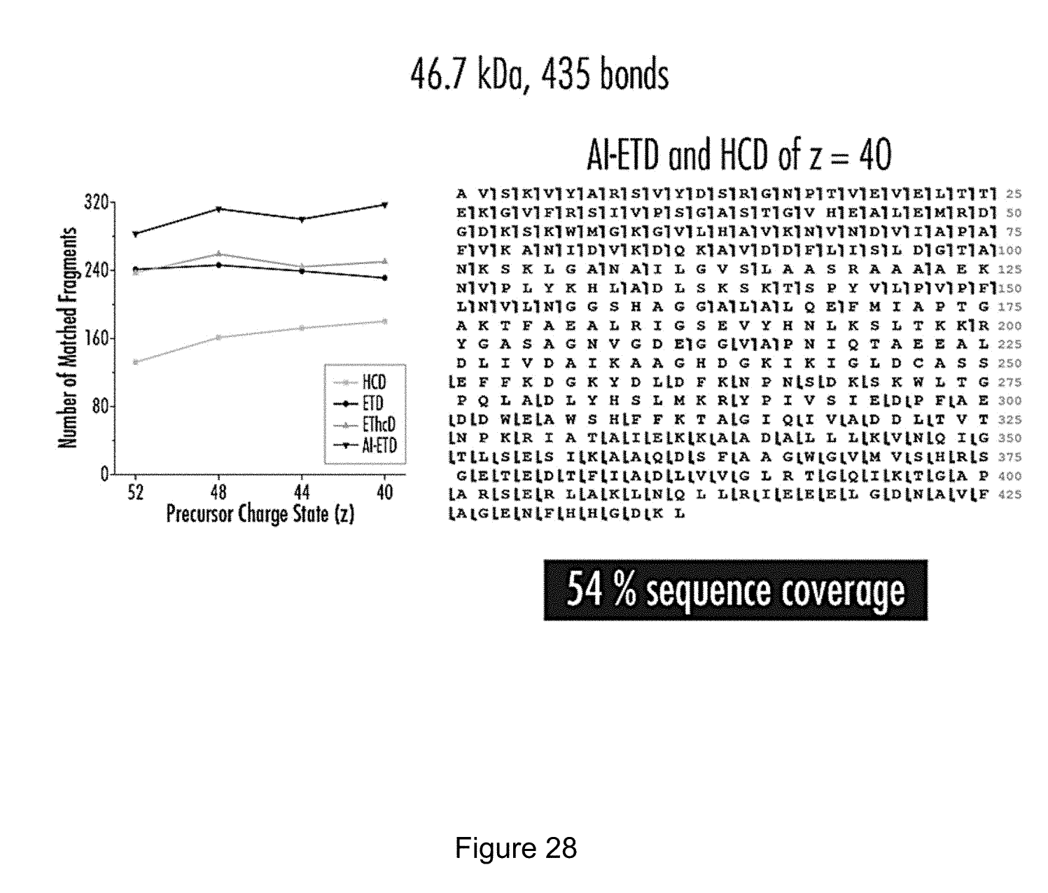

Similar experiments were performed with enolase using a combination of shorter reaction times/lower NCE and longer reaction times/higher NCE (FIG. 27). AI-ETD provided a higher number of matched fragments (FIG. 28).

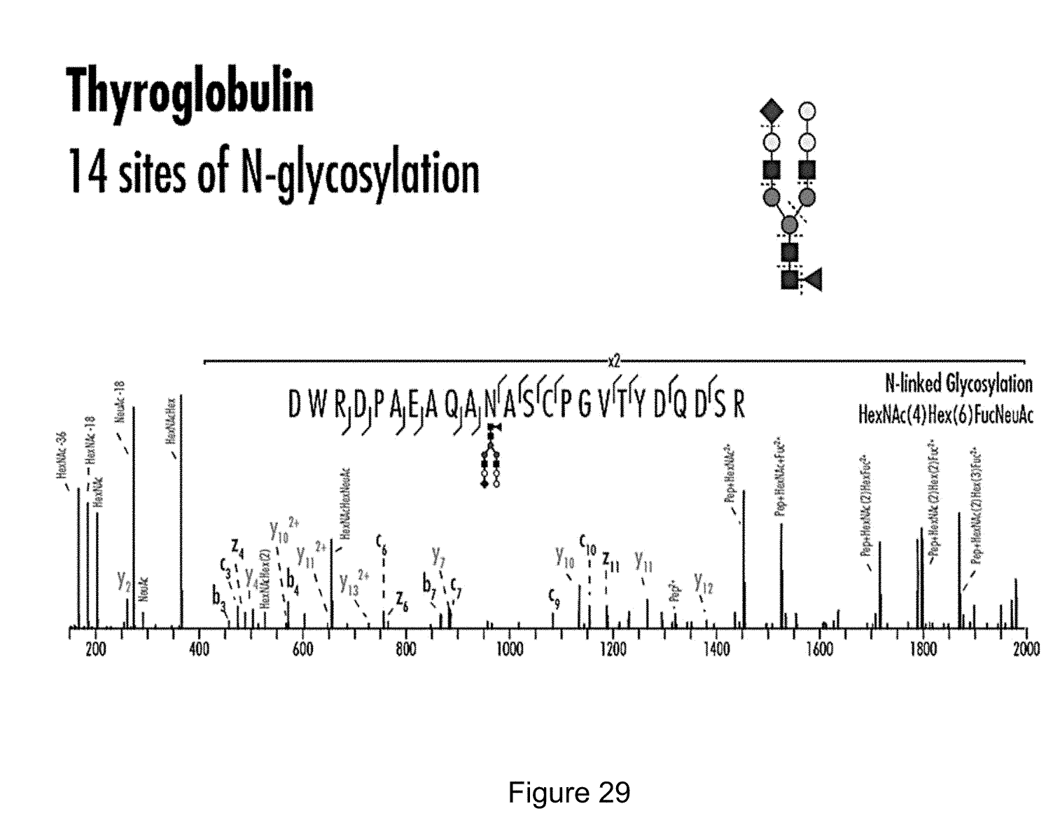

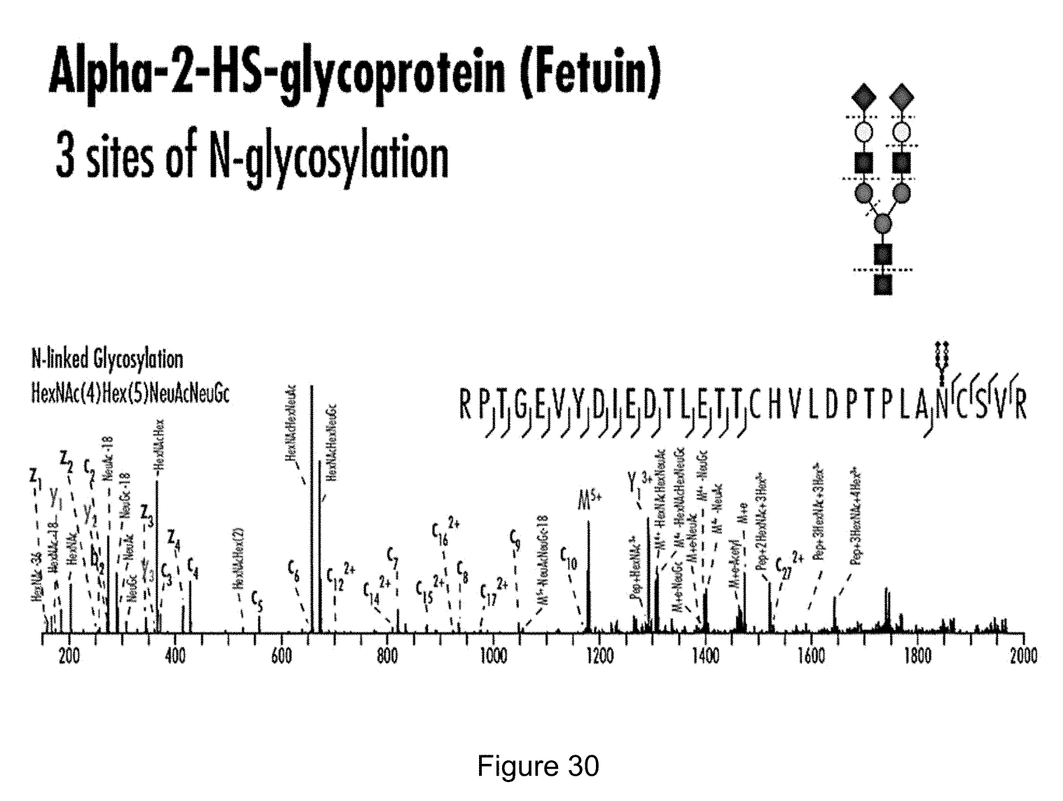

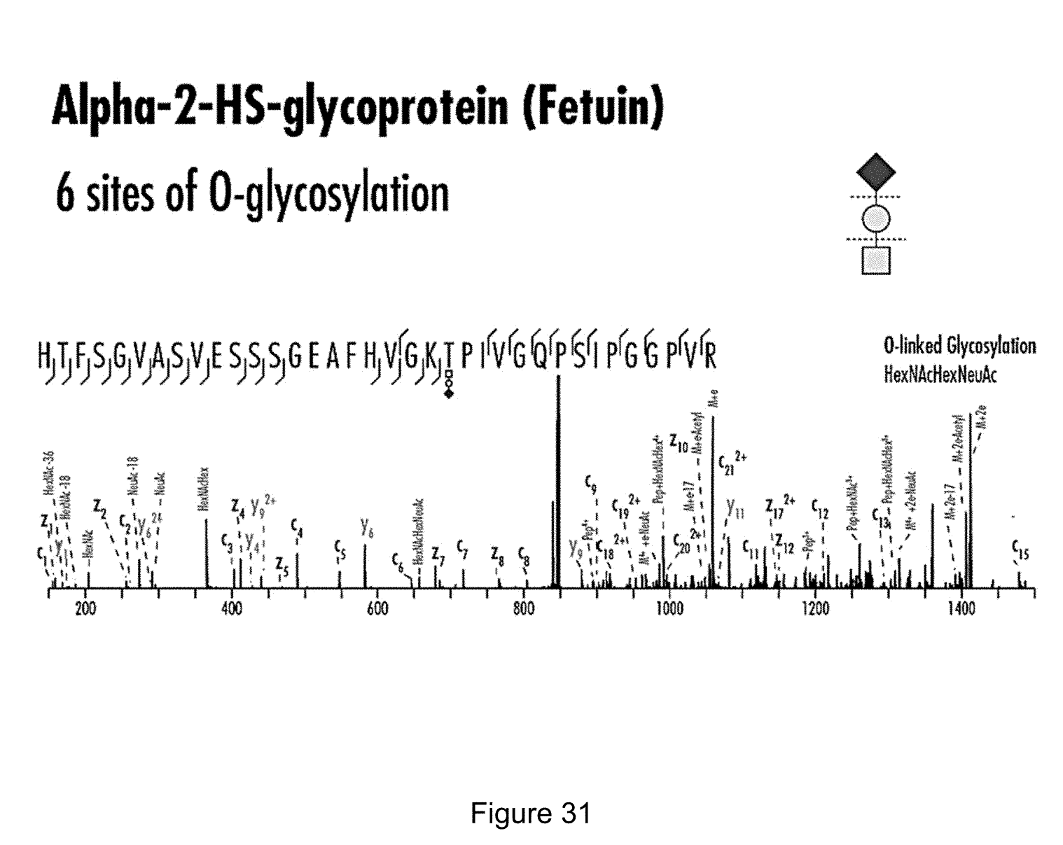

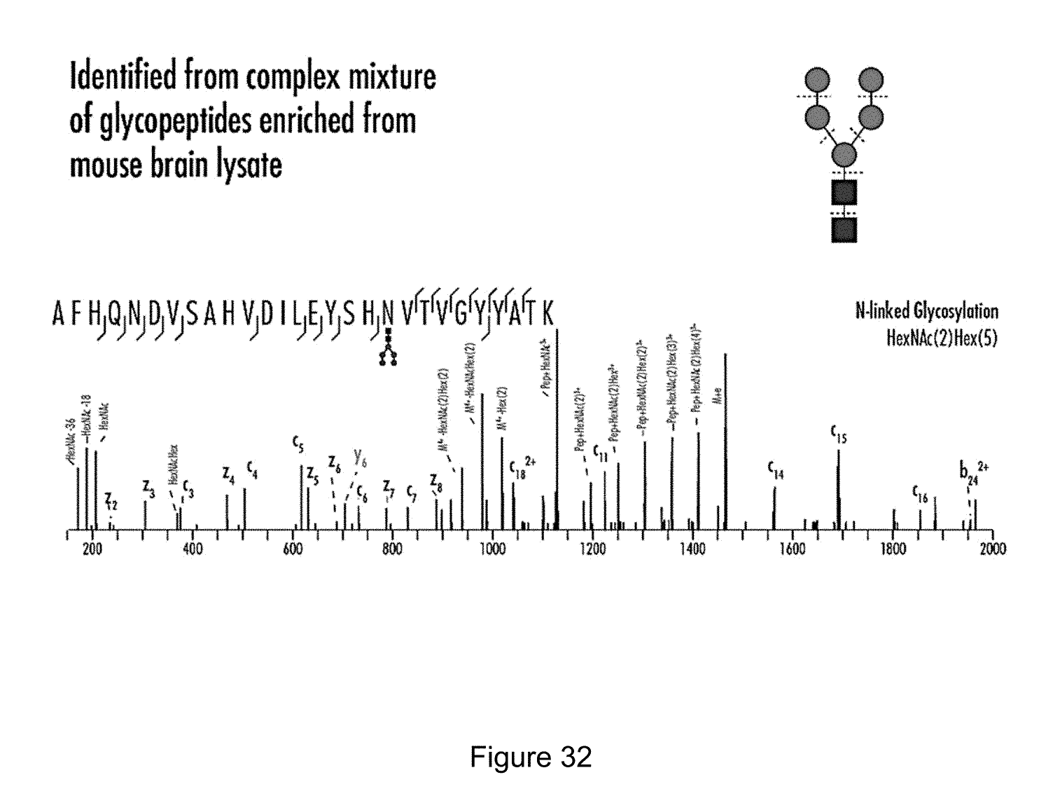

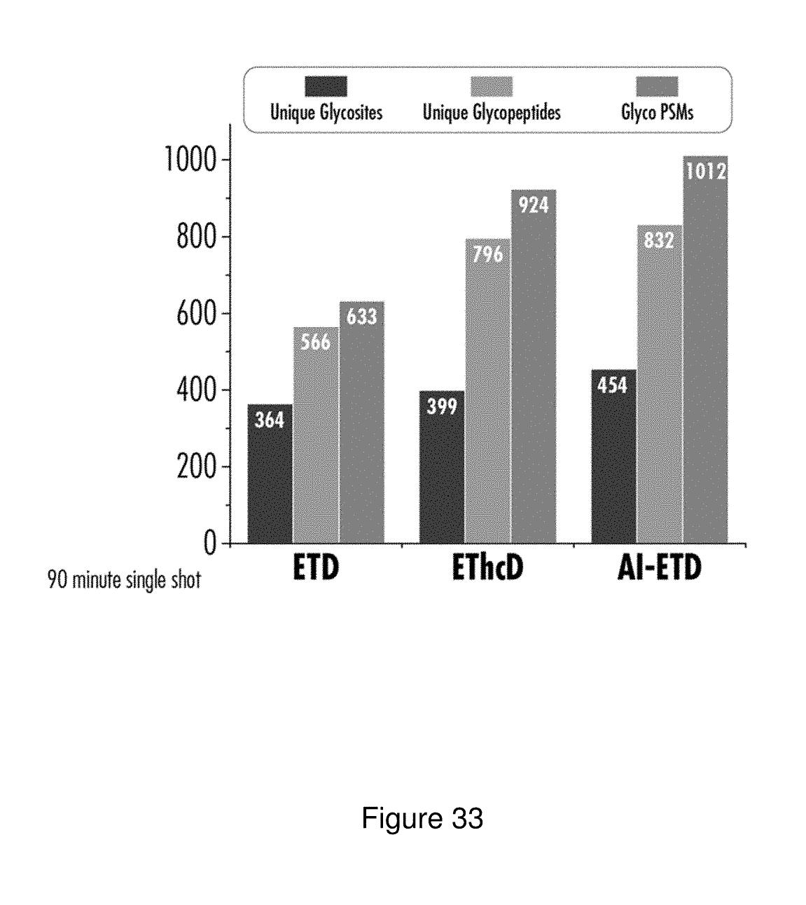

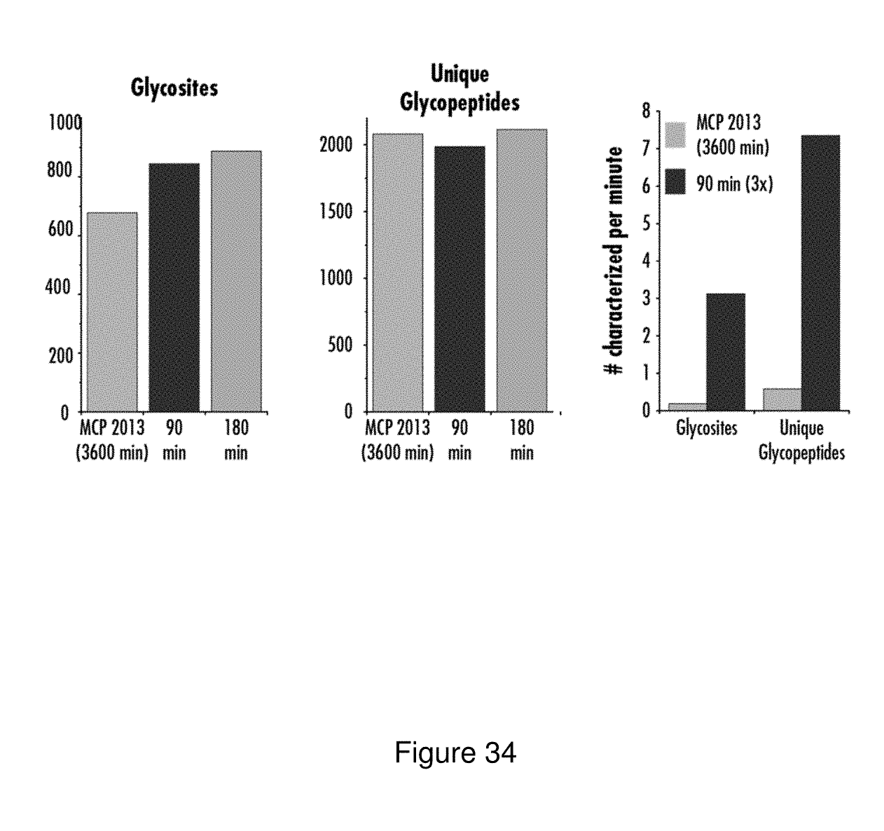

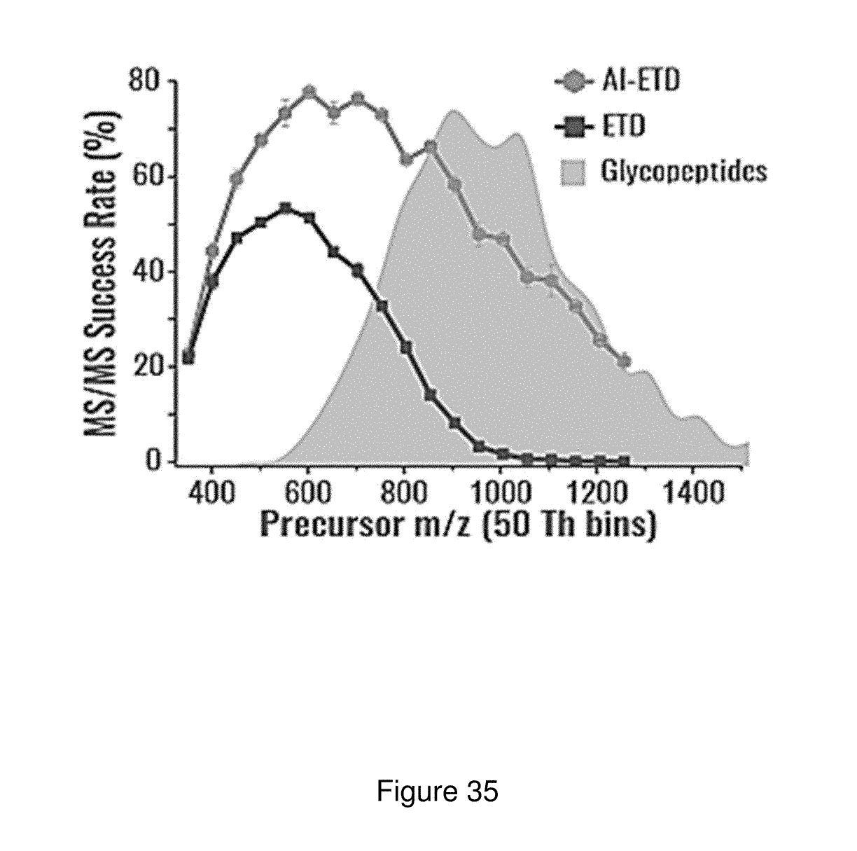

AI-ETD is also able to effectively characterize glycopeptides. As seen in FIGS. 29-32, AI-ETD is able to create fragments isolating sites for N-glycosylation and O-glycosylation. AI-ETD was able to generate a greater number of unique glycosites, glycopeptides, and confidently localized glcyo peptide spectral matches (PSMs) than ETD and EThcD (FIG. 33 and FIG. 34).

### Example 4—Characterization of Glycosylated Peptides

Protein glycosylation is a prevalent, chemically complex, and biologically diverse post-translational modification (PTM) involved in a wide array of intra- and inter-cellular functions. Approximately half of all expressed proteins undergo glycosylation, and this heterogeneous modification accounts for the greatest proteome diversity over any other PTM. Changes in protein glycosylation are associated with cellular proliferation, inter-cellular communication, and metabolic processes, making the characterization of the cellular landscape of protein glycosylation integral to advancing our understanding of cell biology. Glycan microheterogeneity, i.e., different glycans modifying the same glycosite, makes glycan identity at a given site crucial to the biological context of the modification. This unique feature of glycosylation makes analysis of intact glycopeptides imperative for glycoproteome characterization, but the complex nature of glycopeptides limits the ability of current technology to study the glycoproteome in a high-throughput, global manner.

Mass spectrometry (MS) is an ideal platform to advance global glycoproteome profiling. MS offers high sensitivity and throughput, quantification, and the ability to localize modifications to a single amino residue. Tandem mass spectrometry (MS/MS) is the primary MS approach, but standard dissociation methods only access either glycan or peptide information. Collision-based methods, like resonant excitation, access vibrational modes that typically fragment the weakest bonds in a precursor. Glycosidic bonds, between sugar moieties in glycans, are generally more susceptible to these vibrations than peptide bonds. The resulting spectra are largely dominated by glycosidic bond cleavages, providing information to determine glycan composition but leaving the peptide intact and thus unidentified. Beam-type collisional-activation (i.e., higher energy collisional dissociation, HCD) can access some higher energy pathways, in addition to generating fragments from lower energy pathways, but the majority of HCD spectra fail to provide the extensive fragmentation needed for peptide sequence elucidation. An alternative to the collision-based fragmentation is electron-driven dissociation, where radical reactions drive peptide backbone bond fragmentation. The most ubiquitous of the electron-driven dissociation methods is electron transfer dissociation (ETD), with nearly 1,000 commercial Orbitrap mass spectrometer systems in use worldwide. In ETD multiply charged precursor cations are reacted with radical reagent anions. ETD of glycopeptides can generate peptide sequencing ions for confident peptide identification, but the reaction generates very little to no glycan fragments.

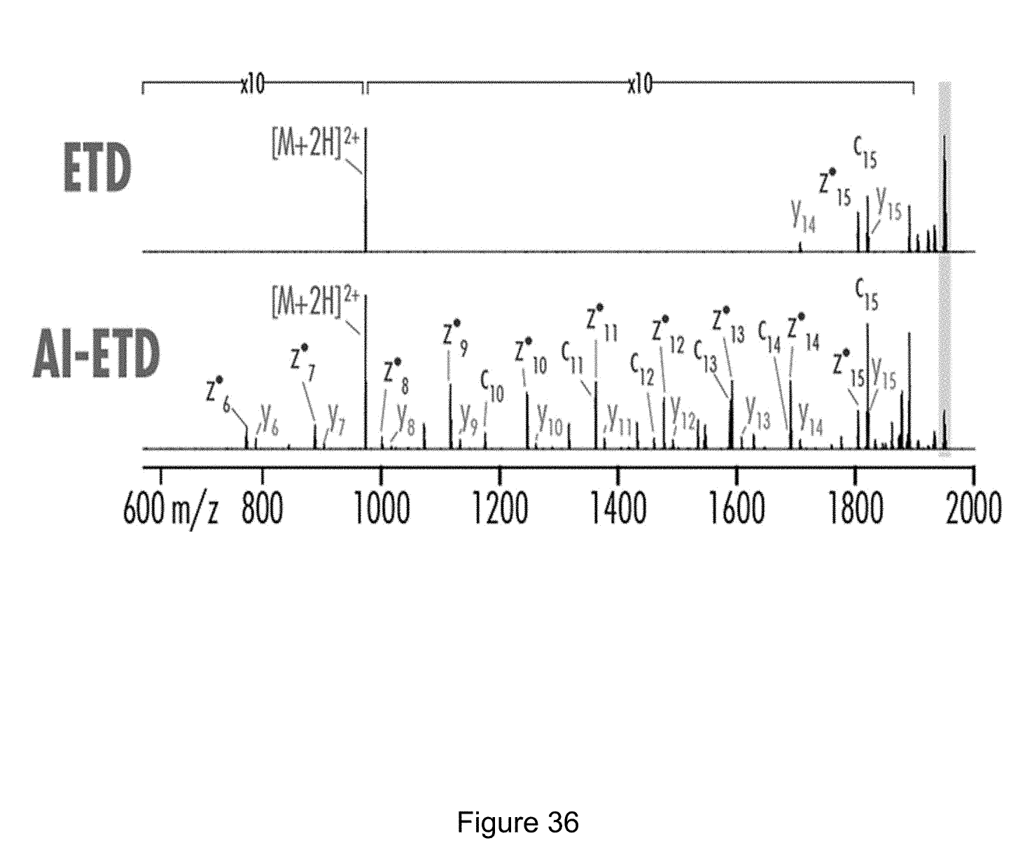

Despite great promise, ETD has had limited success for glycoproteomics. ETD is highly dependent on precursor charge density, which can be measured as a function of precursor m/z (i.e., higher m/z equates to lower charge density). At higher precursor m/z values, ETD fails to generate extensive fragmentation, causing a reduction in identification success rate (FIG. 36). This is largely attributed to more compact gas-phase structures of higher m/z precursors; as charge density decreases, the charge repulsion that contributes to more linear peptide structures at greater charge densities becomes less prevalent and peptides adopt higher order secondary structures. More compact shapes lead to more non-covalent intramolecular interactions between different regions of the peptide backbone. Upon ETD of these more compact peptide precursors, backbone bonds are cleaved to form product ions, but the fragments are held together by non-covalent interactions. These so-called non-dissociative electron transfer (ETnoD) products appear in the spectrum as unfragmented, charge-reduced precursor ions and thus do not contribute any sequence information, limiting the utility of ETD to generate peptide identifications above ˜800 m/z. This problem is exacerbated in glycoproteomic experiments because glycopeptides have significantly higher precursor m/z values. Peptide precursor masses are greatly increased by the addition of glycans, but glycan additions typically do not provide a concomitant increase in charge state (z), contributing to a shift to lower precursor charge densities (gray area FIG. 36) that limits the number of glycopeptides ETD can successfully sequence in glycoproteomic experiments.

AI-ETD for Glycoproteomics.

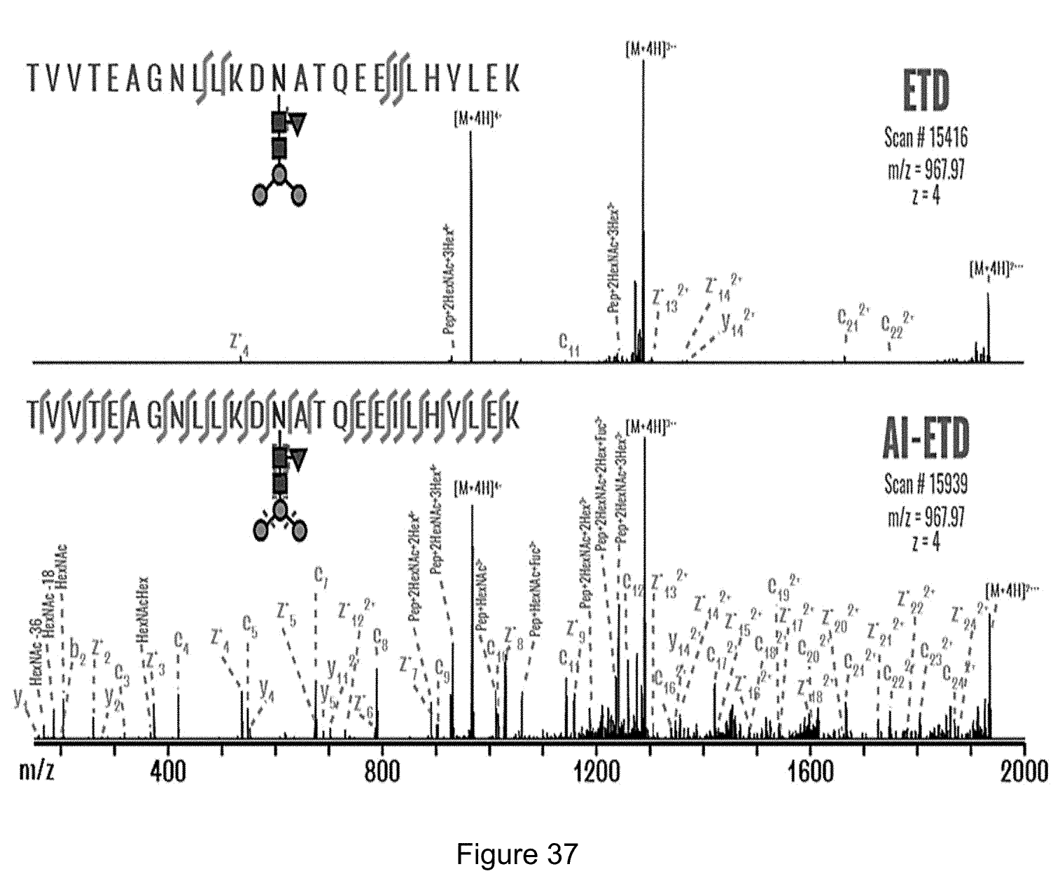

As escribed above, infrared photon activation can be used concurrent with ETD reactions to improve performance for precursors having higher m/z values. This method, called activated-ion ETD (AI-ETD), uses photons to disrupt the non-covalent interactions present in low charge density precursors. By unfolding the peptides as the ETD reaction occurs, ETnoD species are diminished and the efficiency of the reaction (i.e., the amount of precursor converted to product ions) is greatly boosted, significantly increasing the sequence information obtained per spectrum and, thus, MS/MS success rates (top line, FIG. 36). For a precursor having m/z of 900, ETD was able to generate a spectrum that can be mapped to a sequence 10% of the time while AI-ETD was able to be mapped 50% of the time. Several other strategies have been explored to achieve this goal, including collisional activation of ETnoD products, increased temperatures in ETD reaction cells, and photo-activation before, during, and after ETD reactions. Because it prevents the ion-cloud overlap that is essential to ion-ion reactions, collisional activation of the ETD reactants cannot be performed until after the ion/ion reactions are complete. When collisions are used to dissociate ETnoD products, as in ETcaD and EThcD, extensive rearrangements of H atoms occur so that c-type ions become lighter and z-type ions become heavier. These rearrangements create complicated fragment ion isotope distributions that often inhibit successful spectral identification. Post-reaction collisional activation also requires additional steps and time to complete. Photo-activation, however, can be performed simultaneously with the ion-ion reaction by irradiating the trapping volume of the ETD reaction cell, allowing for increased product ion generation with no additional time added to the traditional ETD scan sequence. A new generation quadrupole-Orbitrap-linear ion trap MS system (Orbitrap Fusion Lumos) was modified to perform AI-ETD by affixing a continuous wave CO₂ laser and corresponding beam guiding and focusing optics to the instrument chassis (FIG. 1, panel a). This straight-forward implementation of AI-ETD requires no change to the MS hardware other than the laser addition, and it has shown that it can drastically improve the sequence information obtained in MS/MS scans (FIG. 37) while also considerably outperforming collision-based supplemental activation (i.e., ETcaD and EThcD).

Beyond improving electron-driven peptide sequencing, AI-ETD combines the two complementary dissociation modes that are needed to access glycan and peptide information in intact glycopeptides. Infrared photo-activation provides vibrational excitation that, in addition to improving ETD's ability to cleave the peptide backbone, drives the fragmentation of the glycan's glycosidic bonds. Thus, AI-ETD is unique in that it simultaneously fragments both components of glycopeptides and can provide full sequencing of intact glycopeptides in a single MS/MS scan. Additionally, product ions containing the intact glycan are retained, making glycan identification more straightforward and providing unambiguous glycosite assignment.

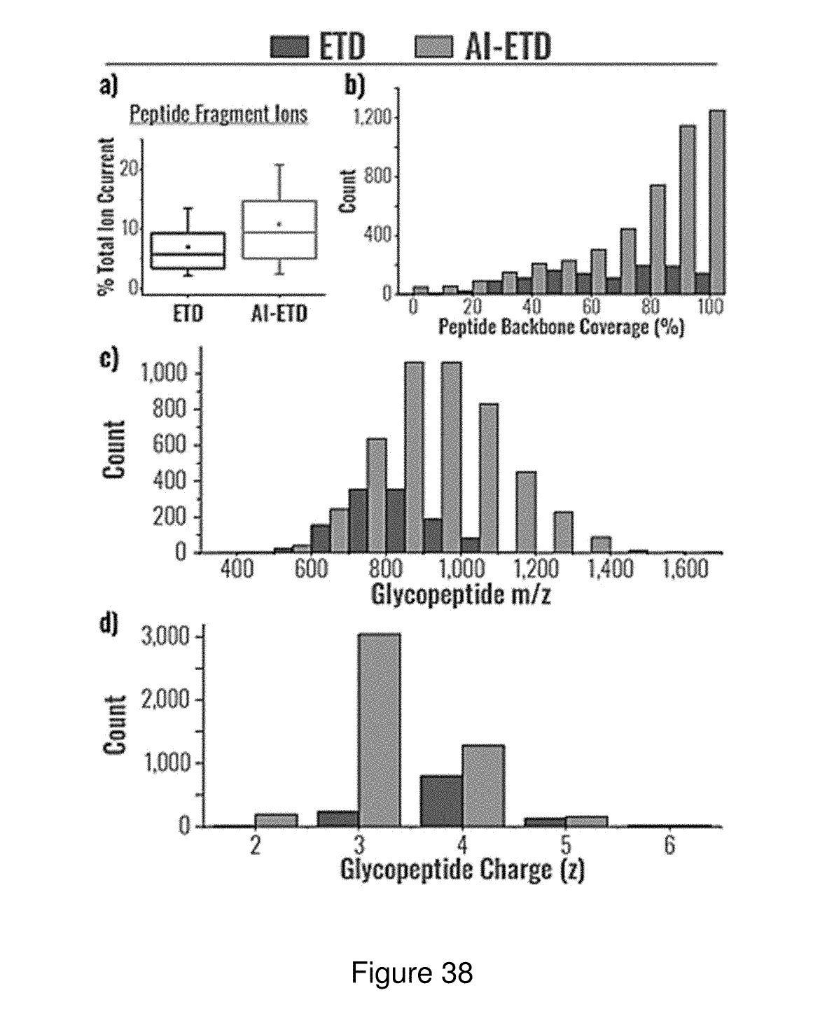

FIG. 38 compares a standard ETD MS/MS scan of an intact glycopeptide with an AI-ETD spectrum of the same precursor. The product ion peaks correspond to peptide-informative product ions while the additionally annotated peaks provide information about the glycan. In the ETD spectrum, a handful of product ions are present, making it possible to assign a peptide sequence, but only ˜17% sequence coverage (4 out of 24 bonds) can be explained by observed products. Additionally, only a fucose loss is observed from the glycan, making glycan identification unlikely. AI-ETD, on the other hand, provides a stark increase in the product ion signal and generates near 100% peptide sequence coverage. The glycosite is clearly deduced from the extensive peptide backbone fragments, including both N- and C-terminal product ions that retain the intact glycan. Additionally, the infrared photo-activation led to the formation of several oxonium ions and Y-type ions (which contain glycan fragments broken along glycosidic bonds still attached to the intact peptide sequence), allowing for more facile glycan identification. Interestingly, this glycopeptide precursor has a relatively high charge state (z=4), which is typically favorable for ETD success. However, with a precursor m/z of 968 Th, the overall moderate charge density leads to only minimal ETD fragmentation for peptide sequencing, underscoring the need for AI-ETD.

Large-Scale Glycopeptide Mapping with AI-ETD.

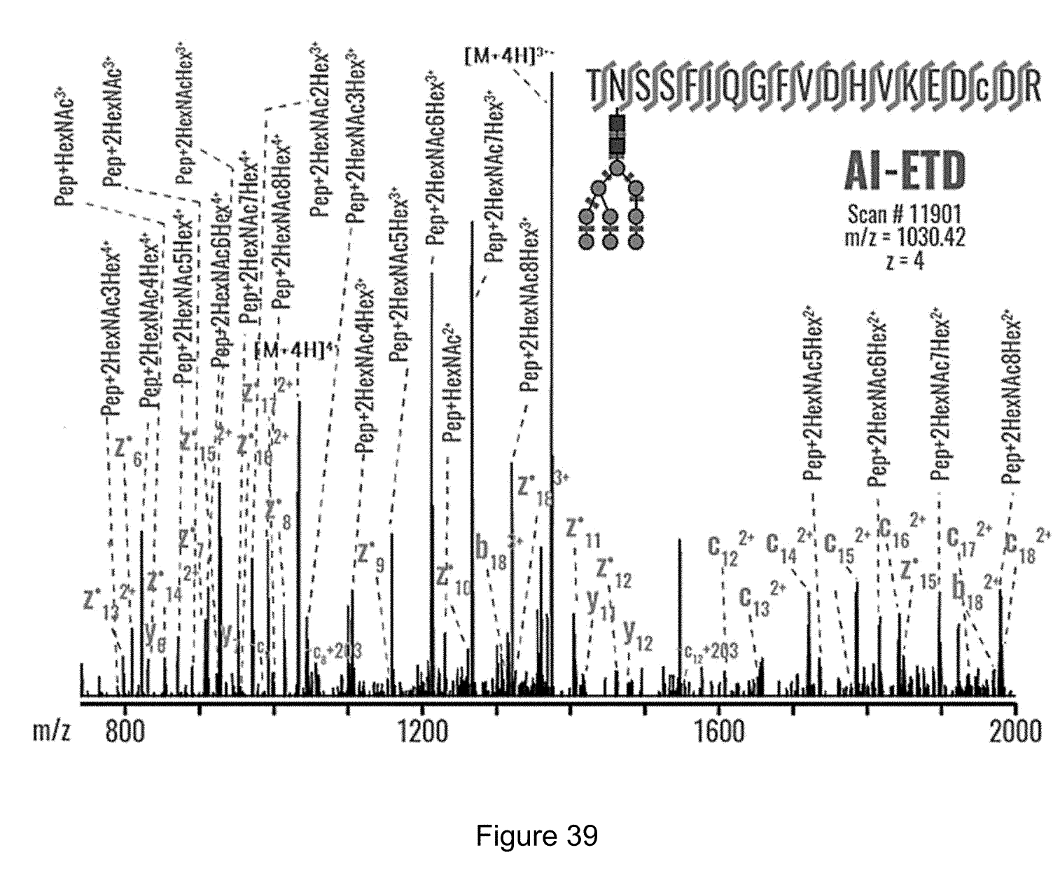

The benefits of AI-ETD became even more pronounced during a global glycoproteomic analysis of glycopeptides enriched from whole cell mouse brain lysates. AI-ETD quadrupled the number of glycopeptide spectral matches sequenced over ETD (4,784 vs. 1,185), providing more signal in peptide-backbone product ion channels and greater peptide sequence coverage than glycopeptides sequenced with ETD. FIG. 39, panel a, shows the distribution of product ion signal corresponding to peptide sequencing ions from the thousands of glycopeptide spectra identified with either technique. Reflecting the example in FIG. 38, AI-ETD funnels more available signal (i.e., total ion current, TIC) into peptide fragment ions for the majority of glycopeptides sequenced. The majority of identified glycopeptides have >80% peptide backbone sequence coverage, enabling confident sequence assignment (FIG. 39, panel b). Interestingly, the shift to higher precursor m/z values in glycoproteomic experiments is difficult to observe from the ETD data alone, simply because so few precursors are successfully sequenced above 1,000 m/z (FIG. 39, panel c). The AI-ETD data, however, shows the majority of glycopeptide precursors exist in the 800-1,200 m/z range, as expected from FIG. 36. The need for AI-ETD is even further pronounced when considering the precursor charge states sequenced between the two methods (FIG. 39, panel d). In standard proteomic experiments, higher charge states 3) are sufficient to give precursors high enough charge density for successful ETD fragmentation. Intact glycopeptides at z=3, however, are rarely sequenced with ETD because the increased mass from the glycan addition decreases overall charge density enough to prevent successful fragmentation. AI-ETD addresses this challenge by mitigating the need for precursors with high charge density. Not only does AI-ETD enable a substantial increase in the number of lower charge density precursors identified (z=2 and 3), but it also improves upon the number of glycopeptides sequenced at higher charge states.

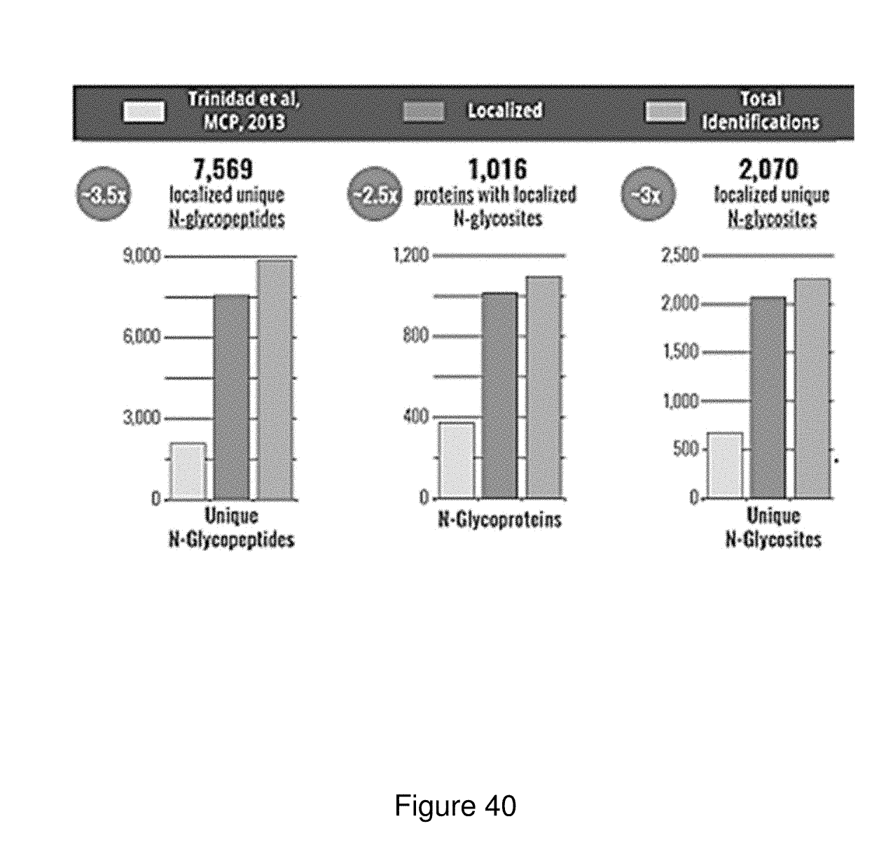

AI-ETD often provides information about glycan composition as well. Glycosidic bonds between the sugar moieties of the glycan are fragmented as the precursors absorb infrared photons and are vibrationally excited. FIG. 40 shows a defined ladder of hexose-loss (162 Da) Y-type ions, leading to a confident identification of HexNAc(2)Hex(9) as the glycan, in addition to several peptide-sequencing product ions retaining the intact glycan. The amount of information provided by AI-ETD in a single scan, both about the peptide sequence and the modifying glycan, is unique. This capability offers new avenues to globally profile complex glycoproteomes in a high-throughput manner. That said, the glycan information obtained is limited to composition only (e.g., the number of hexoses, etc). In general, dissociation of intact glycopeptides does not generate extensive cross-ring fragmentation for deduction of connectivity information or isomer determination (e.g., mannose versus galactose). This is a shortcoming of intact glycopeptide analysis, but there are ways to leverage the type of fragments generated in AI-ETD to narrow the range of possibilities. One way to improve interpretation of the rich fragmentation seen with AI-ETD is to pair high-throughput glycopeptide analyses with more detailed glycomic database generated from MS/MS characterization of released N-glycans. Even without these sample-specific glycan libraries, glycomics data from the literature can be used to construct composition-level databases to use for identifying intact glycopeptides, an approach which has already been used with success.

AI-ETD has enabled the largest global glycoproteomics characterization of a single tissue to date (FIG. 41). The record prior to this work (Trinidad et al., Mol. Cell. Proteomics, 2013, 12: 3474-3488) identified 2,100 N-linked unique glycopeptides from mouse brain tissue in approximately 60 hours of MS-acquisition time using ETD. This study has served as the benchmark for the largest global glycoproteome study in the field. In less analysis time, a 3.5-fold boost was achieved over this landmark result using AI-ETD. With 54 hours of LC-MS/MS analysis time, AI-ETD allowed identification of more than 7,500 unique localized N-glycopeptides from whole cell mouse brain lysate, corresponding to more than 30,000 localized glycopeptide spectral matches (FIG. 41). It should be noted that these identifications passed strict quality filters, including score cutoffs for glycosite localization, with closer to 9,000 total identifications made without the localization criteria. These localized glycopeptides mapped back to 2,070 unique N-glycosites on 1,016 glycoproteins. This depth of glycoproteome characterization was largely enabled by the superior fragmentation and high quality MS/MS spectra generated by AI-ETD, and this method promises to greatly expand the ability of glycoproteomic methods to global profile a range of complex systems.

### Example 5—Modular Implementation of an Infrared Irradiation System

AI-ETD implementation on a pre-existing quadrupole-Orbitrap-quadrupole linear ion trap (q-OT-QLT, Orbitrap Fusion Lumos) can be achieved by employing a CO₂ laser and alignment optics affixed to the outside of the instrument chassis. This approach requires minimal additional hardware, while still allowing for simple laser installation that maintains alignment integrity and robust instrument performance. FIG. 1, panel a, illustrates an overhead view of such a setup, indicating how the laser head is attached to the instrument and how the beam is guided into the dual cell linear ion trap using mirrors and beam steerers. Two focusing lenses were also introduced immediately prior to the ZnSe window placed into the vacuum manifold. The first lens focuses the IR photon beam to ˜1 mm in diameter, while the second lens collimates the beam prior to transmission into the QLT. FIG. 42 (top panel) provides a rendered image of such an implementation. The tube which houses the lenses makes coarse alignment with the QLT straight-forward and the beam steerers allow for simple adjustment of fining tuning of the beam position in the x and y dimensions. Following the hardware modifications in this emboidment, the Lua instrument control software was modified to trigger the laser using TTL logic and a gate controller. To adjust laser power in real-time, a spare instrument DAC was utilized.

This initial implementation setup has many strengths. First and foremost, is the CO₂ laser itself. CO₂ lasers are widespread with many industrial applications. Accordingly, they are mass produced, robust, and affordable. SYNRAD brand lasers have been found to be exceptionally reliable. A downside of this light source, however, is its physical size (approximately 21″×6″×4″). Given its many advantages, it is still beneficial to accommodate the size of the CO₂ laser. Additionally, the combination of mirrors and lenses useful for its implementation (such as illustrated in FIG. 1, panel a) is robust with months of adjustment free operation. Additionally, the optical train provides many degrees of freedom in adjustment enabling a high degree of light delivery optimization. These characteristics are highly beneficial for experimental applications and development. However, IR laser beams are invisible to the human eye and can be very dangerous at the powers employed in embodiments in this invention. The laser head size, combined with mirror and lens placements, make it difficult to develop a “bolt on” module that non-experts can safely install and operate. Additionally, employing multiple adjustable optical components leaves open areas in the IR laser beam path that could cause injury.

Fiber optic coupling will eliminate the current size and safety concerns. Fiber optics can provide convenient, compact, and robust delivery of laser beams. Optical fibers have been available for many years in the visible, near infrared and short-wave infrared wavelength region. A fiber optic analog for radiation in the long-wave infrared region, the type produced by CO₂ lasers (i.e., 10.6 μm), has recently become available. Fiber optics made from chalogenide glasses were the first to appear but they would only transmit efficiently out to 9 μm and were extremely fragile. Hidaka et al. (J. Applied Physics, 1981, 52:4467) proposed using oxide glass to form a hollow core fiber (fiber optic with no central core) to act as a waveguide for CO₂ laser light. This technology has been expanded into the current hollow core glass waveguides which are fabricated by depositing a reflective silver layer followed by a dielectric silver iodide layer inside a hollow capillary tube. The outside of the glass capillary is coated with a polyimide protective jacket making it flexible and robust. This fiber is a commercially available and capable of delivering single mode (i.e., Gaussian beam profile), high powered laser light from a CO₂ laser.

With these new hollow fiber waveguides, the entire AI-ETD optical train was redesigned, allowing placement of the laser head within the instrument chassis. Further, all the existing optical components were reduced to a single lens mounted to a modified vacuum chamber endcap. With this setup, anyone having an ETD-equipped Lumos mass spectrometer can implement AI-ETD by simply replacing the vacuum chamber endcap and making a fiber optic connection to the laser. No specialized expertise is required. Safety concerns are also alleviated here as the entire light delivery system is enclosed—a key step toward commercialization.

FIG. 1, panel b, and FIG. 42, bottom panel, illustrate this implementation of the hollow fiber waveguide. The end cap of the vacuum chamber enclosing the ion trap is replaced with a modified to accept a cylindrical adapter designed to hold a single ZnSe lens and a SMA (SubMiniature version A) connector to which the fiber optic is attached. IR radiation emerging from the fiber will be focused by the single lens into the ion trap where ETD occurs. The lens will also serve as the window between ultra-high vacuum and atmospheric pressure. The prototype adapter allows for adjustment of the distance between the SMA connector and lens so that the optimal focus setting can be determined. Once found, additional adapters manufactured with a fixed distance can be utilized. Alternatively, a modular adapter completely replaces the existing vacuum cover. One end of the hollow core fiber optic is attached to the SMA connector at the adapter and the other end to the SMA connector on the laser, completing the optical setup. The laser can be placed anywhere that is convenient. For example, the laser can be attached to the bottom side of the mass spectrometer support table, providing for complete enclosure of the entire AI-ETD system within the existing spectrometer housing.

It has already been determined that real-time adjustment of laser power in most applications is unnecessary. Accordingly, automated laser power adjustment will typically not be implemented, eliminating the necessity to access a digital to analog converter from the mass spectrometer control software. In such systems, laser power can still be adjustable manually for system optimization as well as the analysis of other sample types requiring different laser power. Triggering for the laser is provided through an existing external signal on the Lumos system. With the design using a hollow-core fiber optic connection, safety concerns are reduced or eliminated, the complexity of the system is simplified, the requirement to modify MS control software is reduced, and the need for custom electronics is removed.

Currently, laser alignment and power delivery are tested by monitoring the dissociation of a common background ion, such as decamethylcyclopentasiloxane (˜371 m/z). As laser power is increased, a methane neutral loss occurs via infrared multiphoton dissociation to give a product ion of ˜355 m/z. When the laser is properly aligned, complete conversion of the precursor to the m/z 355 product ion is accomplished with ˜50 ms of 12-15 W of laser output. Comparison of conversion ratios at various laser powers and irradiation times, to the current implementation, can confirm the effectiveness of the modular hollow-core fiber irradiation system.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A mass spectrometer device for analyzing a sample, the device comprising:
a) an ion source for generating ions from the sample;
b) one or more chambers having an inlet for receiving the ions and having ion pathway optics for transmitting the ions along an ion injection pathway,
c) a mass analyzer in fluid communication with the ion injection pathway;
d) a linear ion trap in fluid communication with the ion injection pathway, wherein said linear ion trap has a longitudinal axis, a first end proximal to the ion source, and a second end distal to the ion source, and wherein said linear ion trap comprises a high pressure cell, a low pressure cell, and a beam entrance window; and
e) an optical assembly positioned external to the linear ion trap and the ion injection pathway, said optical assembly comprising an infrared (IR) laser and one or more optical elements selected from the group consisting of guiding mirrors, waveguides, hollow silica waveguides, optical fibers, beam steerers, and focusing lenses,
wherein said optical assembly is able to provide a photon beam through the beam entrance window into the linear ion trap, wherein the photon beam has an optical axis which is substantially aligned along the longitudinal axis of the linear ion trap.

2. The mass spectrometer device of claim 1 wherein the one or more optical elements comprise a hollow silica waveguide or an optical fiber able to transport photon beams from the IR laser to the linear ion trap.

3. The mass spectrometer device of claim 1 wherein the IR laser is a continuous wave laser.

4. The mass spectrometer device of claim 1 further comprising a chassis encompassing the one or more chambers, ion injection pathway, mass analyzer, and linear ion trap, wherein the IR laser and/or one or more optical elements are rigidly mounted on the chassis.

5. The mass spectrometer device of claim 1 wherein the device does not comprise an additional collision cell or ion trap between the mass analyzer and linear ion trap along the ion injection pathway.

6. The mass spectrometer device of claim 1 wherein the IR laser has a power up to 60 watts.

7. The mass spectrometer device of claim 1 wherein the IR laser has a power between approximately 6 to 30 watts.

8. The mass spectrometer device of claim 1 wherein the mass analyzer is an orbitrap mass analyzer.

9. The mass spectrometer device of claim 1 wherein the IR photon beam is focused to a waist between approximately 0.5 to 2 mm in diameter and then columnated prior to entering the linear ion trap.

10. The mass spectrometer device of claim 1 further comprising a beam dampening barrier which prevents photon beams from passing out of the linear ion trap.

11. The mass spectrometer device of claim 1 further comprising a controller operably connected to the ion injection pathway ion optics and optical assembly; wherein the controller controls the ion injection pathway ion optics and optical assembly so as to:
transmit the ions along a first direction away from the inlet through the ion injection pathway into the high pressure cell and low pressure cell of the linear ion trap;
operate the IR laser to transmit the photon beam into the high pressure cell while said ions are present in the high pressure cell, thereby fragmenting at least a portion of the ions to generate product ions; and
transmit at least a portion of the generated product ions from the linear ion trap to the mass analyzer.

12. The mass spectrometer device of claim 11 wherein the controller controls the ion injection pathway ion optics and optical assembly so as to further:
transmit unfragmented ions and at least a portion of the generated product ions from the high pressure cell to the low pressure cell; and
operate the IR laser to transmit the photon beam into the low pressure cell while said unfragmented ions and generated product ions are present in the low pressure cell, thereby fragmenting additional ions to generate additional product ions.

13. A method for generating product ions from a sample, the method comprising:
a) generating ions from the sample using an ion source;
b) transmitting said ions from said ion source through an inlet into an ion injection pathway of a mass spectrometer device having ion pathway optics and a mass analyzer;
c) transmitting the ions along a first direction away from the inlet through the ion injection pathway into a high pressure cell of a linear ion trap, wherein said linear ion trap has a longitudinal axis, a first end proximal to the ion source, and a second end distal to the ion source, and wherein said linear ion trap comprises the high pressure cell, a low pressure cell, and a beam entrance window, wherein said beam entrance window is positioned in the second end of the linear ion trap;
d) transmitting a first photon beam from an external infrared (IR) laser through the beam entrance window into the high pressure cell while said ions are present in the high pressure cell, thereby fragmenting at least a portion of the ions to generate product ions; and
e) transmitting at least a portion of the generated product ions from the linear ion trap to the mass analyzer of the mass spectrometer device.

14. The method of claim 13 further comprising the steps of:
f) before transmitting the generated product ions to the mass analyzer, transmitting unfragmented ions and at least a portion of the generated product ions from the high pressure cell to the low pressure cell; and
g) transmitting a second photon beam from the external IR laser through the beam entrance window into the low pressure cell while said unfragmented ions and generated product ions are present in the low pressure cell, thereby fragmenting additional ions to generate additional product ions.

15. The method of claim 14 wherein the second photon beam has lower power than the first photon beam.

16. The method of claim 14 wherein the first photon beam has a power between approximately 12 to 24 watts, and the second photon beam has a power between approximately 8 to 10 watts.

17. The method of claim 14 wherein the fragmentation steps using the photon beams from the external IR laser do not increase scanning time of the mass spectrometer device.

18. The method of claim 16 wherein the second photon beam is transmitted into the low pressure trap for a duration of approximately 2 to 10 ms.

19. The method of claim 13 wherein the first photon beam is transmitted into the high pressure trap for a duration of approximately 5 to 200 ms.

20. The method of claim 13 wherein the photon beam is transmitted from the external IR laser to the beam entrance window through one or more hollow silica waveguides or optical fibers.

21. The method of claim 15 wherein said ion source is an electrospray ionization source, a MALDI source, a chemical ionization source, a laser desorption source, a sonic spray source, a photoionization source, a desorption source, or a fast ion bombardment source.

22. The method of claim 13 wherein the sample comprises an unmodified peptide, phosphorylated peptide, glycosylated peptide, isobarically labeled peptide, or an intact protein.

23. The method of claim 13 wherein the sample comprises a glycosylated or phosphorylated peptide.

24. The method of claim 23 wherein the transmitted product ions provide a peptide backbone sequence coverage of at least 50%.

25. The method of claim 13 wherein product ions are generated from a sample having a charge state of +2 or greater.
